(19) United States
(12) Patent Application Publication
(10) Pub. No.: US 2007/0203080 A1
(43) Pub. Date: Aug. 30, 2007

(54) New Drug Delivery System for Crossing the Blood Brain Barrier

(75) Inventor: Bruce H. Lipshutz, Goleta, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: THE REGENTS OF THE UNIVERSITY OF CALIFORNIA, Oakland, CA (US)

(21) Appl. No.: 11/675,622

(22) Filed: Feb. 15, 2007

Related U.S. Application Data
(60) Provisional application No. 60/773,897, filed on Feb. 15, 2006.

(51) Int. Cl. A61K 31/704; A61K 31/7034; A61K 31/7048; A61K 31/522; A61K 31/551; A61K 31/397; A61K 31/473; A61K 31/455; A61K 31/325

(52) U.S. Cl. 514/34; 514/159; 514/210.02; 514/220; 514/317; 514/37; 514/214.01; 514/319; 514/297; 514/493; 514/263.35; 544/276; 536/6.4; 536/16.8; 540/102; 540/200; 540/560; 546/93; 546/104; 546/315; 549/467; 546/205; 560/25

(57) ABSTRACT

New ubiquinol analogs are disclosed, as well as methods of using these compounds to deliver drug moieties to the body.

### CROSS REFERENCE TO RELATED APPLICATIONS

This application claims priority under 35 U.S.C. § 119(e) to U.S. Provisional Patent Application No. 60/773,897 filed Feb. 15, 2006, which application is incorporated herein by reference in its entirety for all purposes.

### BACKGROUND OF THE INVENTION

The ubiquinones, also commonly called coenzyme Qₙ (n=1-12), constitute essential cellular components of many life forms. In humans, CoQ₁₀ is the predominant member of this class of polyprenoidal natural products and is well-known to function primarily as a redox carrier in the respiratory chain (Lenaz, COENZYME Q. BIOCHEMISTRY, BIOENERGETICS, AND CLINICAL APPLICATIONS OF UBIQUINONE, Wiley-Interscience: New York (1985); Trumpower, FUNCTION OF UBIQUINONES IN ENERGY CONSERVING SYSTEMS, Academic Press, New York (1982); Thomson, R. H., NATURALLY OCCURRING QUINONES, 3rd ed., Academic Press, New York (1987); Bliznakov et al., THE MIRACLE NUTRIENT COENZYME Q₁₀, Bantom Books, N.Y. (1987)).

Given the biological activities of the naturally occurring ubiquinones, there is great interest in the art to provide synthetic ubiquinone analogs as well as analogs of the structurally related ubiquinols, which represent the reduced forms of the corresponding ubiquinones. Methods for the synthesis of ubiquinones and ubiquinols as well as their respective analogs are described in U.S. Pat. No. 6,545,184 to Lipshutz et al. and U.S. Patent Application No. 20050148675 to Lipshutz et al. The present invention provides additional ubiquinone and ubiquinol analogs as well as methods for their synthesis and methods of use.

Many research activities are focused on the development of novel routes for the administration of pharmaceutical drugs. Efforts are aimed at optimizing the bioavailability, pharmacokinetics and pharmacodynamics of approved and investigational medicines. Other strategies are aimed at increasing the specificity of drug molecules for their respective site of action within the organism. Those targeted therapies may help to reduce pharamaceutically effective dosages, thereby reducing potential side effects. Other efforts involve the development of effective combination therapies that are associated with reduced drug interaction and adverse events.

A major challenge for the treatment of many central nervous system (CNS) disorders is overcoming the difficulty of delivering therapeutic agents through the blood-brain barrier (BBB), which is a membrane that controls the passage of substances from the blood into the CNS. In its neuroprotective role, the blood-brain barrier functions to hinder the delivery of many potentially important diagnostic and therapeutic agents to the brain. Therapeutic molecules that might otherwise be effective in diagnosis and therapy often do not cross the BBB in adequate amounts. Modalities for drug delivery through the BBB entail disruption of the BBB, either by osmotic means or biochemically by the use of vasoactive substances such as bradykinin. Other strategies to go through the BBB may entail the use of endogenous transport systems, including carrier-mediated transporters such as glucose and amino acid carriers; receptor-mediated transcytosis for insulin or transferrin; and active efflux transporters such as p-glycoprotein. Chemistry-based strategies rely on lipid-mediated drug transport. Lipophilic and hydrophobic molecules of low molecular weight that are known to cross the BBB can be used to transport other molecules through the barrier.

An effective method for the delivery of drug molecules through the BBB and into the CNS would represent a significant advance in the art and provide the opportunity to develop treatment modalities for numerous CNS diseases, such as CNS infections, Parkinson's disease as well as other brain diseases, including brain tumors. The present invention addresses these and other needs.

Another area of interest lies in the effective administration of lipid-altering pharmacological therapies. An elevated concentration of low-density-lipoprotein cholesterol (LDL-C), for example, plays a causal role in the development of conditions such as coronary heart disease and ischemic stroke. For patients who require lipid-lowering treatment, statin monotherapy is an appropriate treatment. However, in those patients where statin monotherapy does not produce optimal lipid levels, the combination of a statin with niacin, a bile acid sequestrant, a fibric acid derivative, or a cholesterol absorption inhibitor may provide improved control. Introduction of novel therapies, such cholesteryl ester transfer protein (CETP) inhibitors, and cholesterol acyltransferase (ACAT) inhibitors provide additional opportunities for the development of combination strategies aimed at optimizing lipid profiles in patients who are currently difficult to treat. The choice of combination therapy depends upon the patient's lipid profile and tolerability of the medication.

Novel methods for the systemic delivery of lipid lowering drug molecules that positively affect the pharmaceutical properties of said drug molecules, and help to minimize drug interactions, adverse events and pharmaceutically effective dosages of these drugs would represent a significant advance in the art and provide the opportunity to develop improved treatments for associated diseases. The present invention addresses these and other needs.

### SUMMARY OF THE INVENTION

The present invention provides ubiquinol analogs comprising a functional moiety, such as a drug moiety. Those analogs can be used in the methods of the invention.

In a first aspect, the invention provides a compound according to Formula (V):

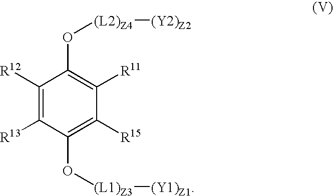

In Formula (V), R¹¹, R¹² and R¹³ are members independently selected from H, halogen, CN, substituted or unsubstituted alkyl, substituted or unsubstituted heteroalkyl, substituted or unsubstituted aryl, substituted or unsubstituted heteroaryl and substituted or unsubstituted heterocycloalkyl. R¹² and R¹³, together with the carbon atoms to which they are attached, are optionally joined to form a 5- to 7-membered ring.

Y1 and Y2 are members independently selected from H, a labeling moiety, a targeting moiety, and a drug moiety. In a preferred embodiment at least one member selected from Y1 and Y2 is a member selected from a labeling moiety, a targeting moiety and a drug moiety. In a particularly preferred embodiment, at least one of Y1 and Y2 is a drug moiety. In a further preferred embodiment, Y1 and Y2 are not both a polymeric hydrophilic moiety selected from a polyether and a polyalcohol, and when one member of Y1 and Y2 is a polyether or a polyalcohol, the other member is selected from a labeling moiety, a targeting moiety and a drug moiety.

Z1, Z2, Z3 and Z4 are members independently selected from 0 and 1 with the proviso that at least one member selected from Z1 and Z2 is 1. When Z4 and Z2 are both 0, (L2)_{Z}4-(Y2)_{Z}2 is preferably a member selected from H, a negative charge and a salt counterion; and when Z3 and Z1 are both 0, (L1)_{Z}3-(Y1)_{Z}1 is preferably a member selected from H, a negative charge and a salt counterion. L1 and L2 are independently selected cleavable linker moieties.

R¹⁵ is a member selected from branched, unsaturated alkyl, —C(O)H, and —CH₂Z^{a} wherein Z^{a} is a member selected from R¹⁷, OR¹⁷, SR¹⁷, NR¹⁷R¹⁸ and a leaving group, wherein R¹⁷ and R¹⁸ are members independently selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl.

In another aspect the invention provides a method of treating a neurological disorder. The method includes administering to a subject in need of such treatment, a therapeutically effective amount of a compound of the invention or a pharmaceutically acceptable salt or solvate thereof.

In yet another aspect, the invention provides a method of increasing the concentration of a drug molecule in the tissue of the brain. The method includes administering to a subject in need of such treatment, a therapeutically effective amount of a compound of the invention or a pharmaceutically acceptable salt or solvate thereof wherein the compound comprises the drug molecule.

In a further aspect, the invention provides a method of enhancing the therapeutic activity of a drug molecule. The method includes providing a compound of the invention which includes the drug molecule and administering a therapeutically effective amount of the compound to a subject in need of such treatment.

In another aspect the invention provides a method of lowering the cholesterol concentration in the bloodstream of a patient. The method includes administering to a subject in need of such treatment, a pharmaceutically effective amount of a compound of the invention or a pharmaceutically acceptable salt or solvate thereof wherein the compound includes at least one drug moiety selected from a CETP inhibitor and a statin.

In another aspect, the invention provides a method of lowering the concentration of low-density lipoprotein (LDL) in the bloodstream of a patient. The method includes administering to a subject in need of such treatment, a pharmaceutically effective amount of a compound of the invention or a pharmaceutically acceptable salt or solvate thereof, wherein the compound includes at least one drug moiety that is a statin.

In a further aspect, the present invention provides a pharmaceutical composition comprising a compound of the invention or a pharmaceutically acceptable salt thereof and a pharmaceutically acceptable carrier.

Other objects and advantages of the invention will be apparent to those of skill in the art from the detailed description that follows.

### DETAILED DESCRIPTION OF THE INVENTION AND THE PREFERRED EMBODIMENTS

I. Definitions

The term “alkyl,” by itself or as part of another substitutent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substitutent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and CH═CH—N(CH₃)—CH₃. Up to two heteroatoms may be consecutive, such as, for example, —CH₂—NH—OCH₃ and —CH₂—O—Si(CH₃)₃. Similarly the term “heteroalkylene” by itself or as part of another substitutent means a divalent radical derived from heteroalkyl, as exemplified, but not limited by, —CH₂—CH₂—S—CH₂—CH₂— and CH₂—S—CH₂—CH₂—N₁—CH₂-. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′-represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively, Additionally, for heterocycloalkyl, a heteroatom can occupy the position at which the heterocycle is attached to the remainder of the molecule, Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1 piperazinyl, 2-piperazinyl, and the like.

The terms “halo” or “halogen,” by themselves or as part of another substitutent, mean, unless otherwise stated, a fluorine, chlorine, bromine, or iodine atom. Additionally, terms such as “fluoroalkyl,” are meant to include monofluoroalkyl and polyfluoroalkyl.

The term “aryl,” employed alone or in combination with other terms (e.g., aryloxy, arylthioxy, arylalkyl) means, unless otherwise stated, an aromatic substitutent which can be a single ring or multiple rings (up to three rings), which are fused together or linked covalently. “Heteroaryl” are those aryl groups having at least one heteroatom ring member. Typically, the rings each contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substitutents described below. The term “arylalkyl” is meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl,” “heteroalkyl” and “aryl”) are meant to include both substituted and unsubstituted forms of the indicated radical. Preferred substitutents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substitutents,” and they can be one or more of a variety of groups selected from, but not limited to: substituted or unsubstituted alkyl, substituted or unsubstituted heteroalkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, -SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, erg, aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substitutents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substitutents described for the alkyl radical, substitutents for the aryl and heteroaryl groups are generically referred to as “aryl group substitutents.” The substitutents are selected from, for example: substituted or unsubstituted alkyl, substituted or unsubstituted heteroalkyl, substituted or unsubstituted aryl, substituted or unsubstituted heteroaryl, substituted or unsubstituted heterocycloalkyl, —OR′, ═O, ═NR′, —N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substitutents on adjacent atoms of the aryl ring may optionally be replaced with a substitutent of the formula -T-C(O)—(CH₂)_{q}—U—, wherein T and U are independently —NM—, —O—, —CH₂— or a single bond, and the subscript q is an integer of from 0 to 2. Alternatively, two of the substitutents on adjacent atoms of the aryl ring may optionally be replaced with a substitutent of the formula -A-(CH₂)ᵣ—B—, wherein A and B are independently —CH₂—, —O—, —NH—, —S—, —S(O)—, —S(O)₂—, —S(O)₂NR′- or a single bond, and r is an integer of from 1 to 3. One of the single bonds of the new ring so formed may optionally be replaced with a double bond. Alternatively, two of the substitutents on adjacent atoms of the aryl ring may optionally be replaced with a substitutent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substitutent R′ in —NR′- and —S(O)₂NR′- is selected from hydrogen or unsubstituted (C₁-C₆)alkyl.

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “leaving group” refers to a portion of a substrate that is cleaved from the substrate in a reaction. The leaving group is an atom (or a group of atoms) that is displaced as stable species taking with it the bonding electrons. Typically the leaving group is an anion (e.g., Cl⁻) or a neutral molecule (e.g., H₂O). Exemplary leaving groups include a halogen, OC(O)R⁶⁵, OP(O)R⁶⁵R⁶⁶, OS(O)R⁶⁵, and OSO₂R⁶⁵. R⁶⁵ and R⁶⁶ are members independently selected from substituted or unsubstituted alkyl, substituted or unsubstituted aryl, substituted or unsubstituted heteroaryl and substituted or unsubstituted heterocycloalkyl. Useful leaving groups include, but are not limited to, other halides, sulfonic esters, oxonium ions, alkyl perchlorates, sulfonates, e.g., arylsulfonates, ammonioalkanesulfonate esters and alkylfluorosulfonates, phosphates, carboxylic acid esters, carbonates ethers, and fluorinated compounds (e.g., triflates, nonaflates, tresylates), S R⁶⁵, R⁶⁵)₃P⁺, (R⁶⁵)₂S⁺, P(O)N(R⁶⁵)₂(R⁶⁵)₂, P(O)XR⁶⁵X′R⁶⁵ in which each R⁶⁵ is independently selected from the members provided in this paragraph and X and X′ are S or O. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, 3rd ed., John Wiley & Sons, New York, 1999.

The term “labeling moiety” refers to a moiety, which provides a signal that is detectable by a detection method known in the art. The signal can be used to determine the location or concentration of the labeling moiety, for example, in an organism, a tissue sample or a reaction vial. Exemplary signals include color, emitted light of any wavelength, radioactivity, or any other electromagnetic or quantum mechanical effect. Exemplary labeling moieties include but are not limited to fluorescent molecules (e.g. fluorescein), luminescent moieties (e.g., transition-metal complexes), chemoluminescent molecules, molecules used in calorimetric applications (i.e. dye molecules), histochemical staining reagents, photoaffinity labels, magnetic resonance imaging (MRI) agents, radioactive labels, radiotracers and agents used in positron emission tomography (PET).

The term “targeting moiety” refers to a moiety which is capable of binding to a particular tissue- or cell-type (e.g., tumor cells, neuronal or glial cells, liver cells, and the like) with at least some level of specificity. Exemplary targeting moieties are selected from carbohydrates, proteins, peptides, antibodies, and small-molecule ligands. In an exemplary embodiment, the targeting moiety is a ligand for a biological receptor, such as a cell surface receptor. In another exemplary embodiment, the targeting moiety is an antibody that is capable of binding to an antigen, such as a tissue- or tumor-specific antigen.

The term “drug moiety” refers to pharmaceutical drugs and other biologically active molecules. “Drug moiety” includes small-molecule drugs as well as biologics, including peptides, mutant and wild-type polypeptides, mutant and wild-type proteins, antibodies (e.g., humanized, monoclonal antibodies) and the like.

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. The ring optionally included a heteroatom. Thus, the term “5- to 7-membered ring” includes, for example pyridinyl and piperidinyl. The term “ring” further includes a ring system comprising more than one “ring”, wherein each “ring” is independently defined as above.

“Adsorbent”, as used herein refers to a material with the property to hold molecules of fluids without causing a chemical or physical change. Examples are Silica gel, Alumina, Charcoal, Ion exchange resins and others, characterized by high surface/volume ratio.

As used herein, the term “acyl” describes a substitutent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si) and boron (B).

The symbol “R” is a general abbreviation that represents a substitutent group that is selected from substituted or unsubstituted alkyl, substituted or unsubstituted heteroalkyl, substituted or unsubstituted aryl, substituted or unsubstituted heteroaryl, and substituted or unsubstituted heterocycloalkyl groups.

The phrase “therapeutically effective amount” as used herein means that amount of a compound, or composition comprising a compound of the present invention which is effective for producing some desired therapeutic effect at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in human beings and animals without excessive toxicity, irritation, allergic response, or other problem or complication, commensurate with a reasonable benefit/risk ratio.

The phrase “pharmaceutically acceptable carrier” as used herein means any pharmaceutically acceptable material, which may be liquid or solid. Exemplary carriers include vehicles, diluents, additives, liquid and solid fillers, excipients, solvents, solvent encapsulating materials. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc, (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The term “pharmaceutically acceptable salts” includes salts of the active compounds which are prepared with relatively nontoxic acids or bases, depending on the particular substitutents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66; 1-19 (1977)). Certain specific compounds of the present invention contain both basic and acidic functionalities that allow the compounds to be converted into either base or acid addition salts.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, sulfamate, bisulfate, phosphate, nitrate, acetate, valerate, oleate, palmitate, stearate, laurate, benzoate, lactate, tosylate, citrate, maleate, ascorbate, palmitate, fumarate, succinate, tartrate, napthylate, mesylate, hydroxymaleate, phenylacetate, glutamate, glucoheptonate, salicyclate, sulfanilate, 2-acetoxybenzoate, methanesulfonate, ethane disulfonate, oxalate, isothionate, lactobionate, and laurylsulphonate salts and the like. See, for example, Berge et al. (1977) “Pharmaceutical Salts”, J. Pharm. Sci. 66:1-19.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention. “Compound or a pharmaceutically acceptable salt or solvate of a compound” intends the inclusive meaning of “or”, in that a material that is both a salt and a solvate is encompassed.

Certain compounds of the present invention possess asymmetric carbon atoms (optical centers) or double bonds; the racemates, diastereomers, geometric isomers and individual isomers are encompassed within the scope of the present invention. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The graphic representations of racemic, ambiscalemic and scalemic or enantiomerically pure compounds used herein are taken from Maehr, J. Chem. Ed., 62; 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “neurological disorder” includes disorders that affect the central nervous system, the peripheral nervous system and the autonomic nervous system.

The term “central nervous system disorder” refers to any abnormal condition of the central nervous system of a mammal. Central nervous system disorder includes neurodegenerative diseases such Alzheimer's disease and Parkinson's disease, neuropsychiatric diseases (e.g. schizophrenia), anxieties, sleep disorders, depression, dementias, movement disorders, psychoses, alcoholism, post-traumatic stress disorder and the like. “Central nervous system disorder” also includes any condition associated with the disorder, such as loss of memory and/or loss of cognition. For instance, a method of treating a neurodegenerative disease would also include treating or preventing loss of neuronal function characteristic of such disease.

II. Introduction

The present invention provides novel ubiquinone analogs and reduced ubiquinone (ubiquinol) analogs and methods for the preparation of these molecules. The invention further provides ubiquinol analogs comprising a functional moiety, such as a drug moiety and methods of use for said analogs.

III. Compositions

III.a.) Ubiquinol Analogs

In a first aspect, the invention provides compounds according to Formula (V):

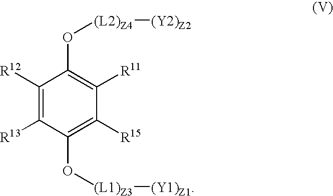

In Formula (V), R¹¹, R¹² and R¹³ are members independently selected from aryl group substitutents In an exemplary embodiment, R¹¹, R¹² and R¹³ are members independently selected from H, halogen, CN, substituted or unsubstituted alkyl, substituted or unsubstituted heteroalkyl, substituted or unsubstituted aryl, substituted or unsubstituted heteroaryl and substituted or unsubstituted heterocycloalkyl. R¹² and R¹³, together with the carbon atoms to which they are attached, are optionally joined to form a 5- to 7-membered ring. In one embodiment, R¹² and R¹³, together with the carbon atoms to which they are attached, are joined to form a substituted or unsubstituted phenyl ring. In another embodiment, R¹² and R¹³ are part of a fused ring system.

In one embodiment, Y1 and Y2 are members independently selected from U and a functional moiety with the proviso that at least one of Y1 and Y2 is a functional moiety. Exemplary functional moieties include labeling moieties, targeting moieties and drug moieties.

In another embodiment, Y1 and Y2 are members independently selected from H, a labeling moiety, a targeting moiety, and a drug moiety. In a preferred embodiment, at least one member selected from Y1 and Y2 is a member selected from a labeling moiety, a targeting moiety and a drug moiety. In a particularly preferred embodiment, at least one of Y1 and Y2 is a drug moiety. In a further preferred embodiment, Y1 and Y2 do not both consist of a hydrophilic polymeric moiety selected from a polyether and a polyalcohol; and when one member selected from Y1 and Y2 consists of a polyether or a polyalcohol, then the other member is selected from a labeling moiety, a targeting moiety and a drug moiety. Exemplary polyethers are polyalkylene glycols, which include polymers of lower alkylene oxides, in particular polymers of ethylene oxide (polyethylene glycols) and propylene oxide (polypropylene glycols).

Z1, Z2, Z3 and Z4 are members independently selected from 0 and 1 with the proviso that at least one member selected from Z1 and Z2 is 1. When Z4 and Z2 are both 0, (L2)_{Z}4—(Y2)_{Z}2 is preferably a member selected from H, a negative charge and a salt counterion (such as Na⁺ or Ca²⁺). Analogously, when Z3 and Z1 are both 0, (L1)_{Z}3—(Y1)_{Z}1 is preferably a member selected from H, a negative charge and a salt counterion. L1 and L2 are independently selected cleavable linker moieties described below (e.g., ester groups).

R¹⁵ is a member selected from branched, unsaturated alkyl, —C(O)H, and —CH₂Z^{a} wherein Z^{a} is a member selected from R¹⁷, OR¹⁷, SR¹⁷, NR¹⁷R¹⁸ and a leaving group, wherein R¹⁷ and R¹⁸ are members independently selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl. In an exemplary embodiment, Z^{a} is halogen, such as Cl.

In another exemplary embodiment, R¹¹ and R¹² are members independently selected from substituted or unsubstituted C₁-C₆ alkyl and substituted or unsubstituted C₁-C₆ alkoxy. In another embodiment R¹¹ is selected from H and methyl. In a particularly preferred embodiment, R¹¹ is selected from H and methyl and R¹² and R¹³ are members independently selected from unsubstituted C₁-C₆ alkyl and unsubstituted C₁-C₆ alkoxy.

In another embodiment, one or more of the substitutents R¹¹, R¹² and R¹³ include halogen atoms to form, e.g., halogen substituted alkyl or halogen substituted alkoxy groups. In one example, at least one of R¹, R² and R³ is halogen-substituted C₁-C₆ alkyl or halogen-substituted C₁-C₆ alkoxy. In another example, the halogen is fluoro. Exemplary fluoroalkyl and fluoroalkoxy groups according to this embodiment of the invention include but are not limited to CF₃, OCF₃, CHF₂, OCHF₂, CH₂F, and OCH₂F.

In a further exemplary embodiment, R¹⁵ includes a structure according to Formula (VI): 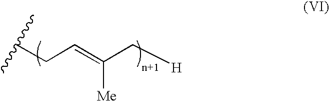
wherein n is an integer selected from 0 to 13. In a preferred embodiment, n is selected from 3 to 11. More preferably, n is selected from 6 to 9. In a particularly preferred embodiment, n is 9.

In one embodiment, one of the phenolic hydroxy groups of the ubiquinol analog is derivatized with a labeling moiety, a targeting moiety or a drug moiety. Exemplary compounds have the structure: 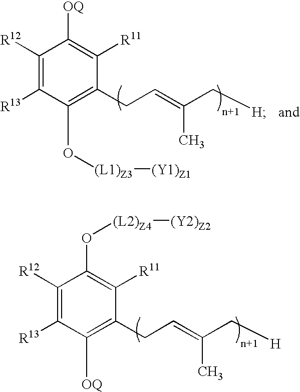
wherein Q is a member selected from H, a negative charge and salt counter ion.

In another aspect, the invention provides mixtures of compounds, in which the mixture includes at least two different compounds of the invention, which may be regioisomers. In an exemplary embodiment, the mixture includes one or more compound, which carrys two drug molecules, as well as one or more compound, which carries only one drug molecule.

Functional Moieties

Preferred compounds of the invention include at least one functional moiety. The term “functional moiety” includes labeling moieties, targeting moieties, drug moieties and any other moiety, which is useful to add function to the ubiquinol analog of the invention. For example, when the molecule includes a therapeutic protein or polypeptide, a polymeric moiety (such as PEG) may be added to the opposite site of the molecule in order to affect bioavailability, pharmacokinetics and/or pharmacodynamics of the resulting ubiquinol analog.

Suitable labeling moieties include but are not limited to fluorescent molecules (e.g. fluorescein), luminescent transition-metal complexes, chemoluminescent molecules, molecules used in colorimetric applications (i.e. dye molecules), histochemical staining reagents, photoaffinity labels, magnetic resonance imaging (MRI) agents, radioactive labels and radiotracers, as well as positron emission tomography (PET) agents.

Suitable targeting moieties are selected from carbohydrates, proteins, peptides, antibodies, and small-molecule ligands. In an exemplary embodiment, the targeting moiety is a ligand for a biological receptor, such as a cell surface receptor. In another exemplary embodiment, the targeting moiety is an antibody that is capable of binding to an antigen, such as a tissue- or tumor-specific antigen. In another exemplary embodiment, compounds according to Formula (V) include a targeting moiety and a drug moiety.

In a preferred embodiment, at least one of the linker moieties L1 and L2 is cleavable in vivo. Exemplary compounds according to this embodiment include one of the following moieties: 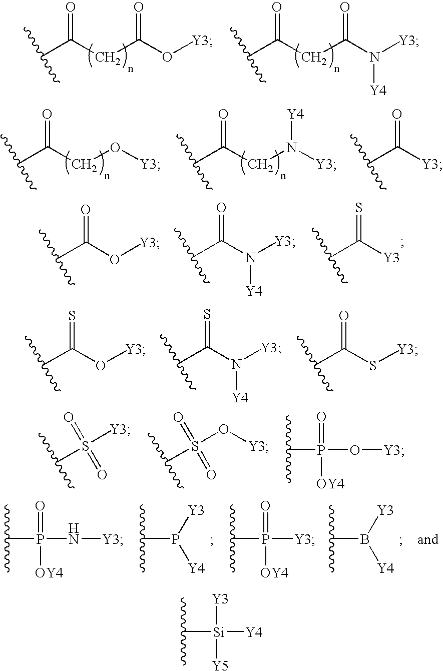
wherein Y3 is a member selected from Y1 and Y2. Y4 and Y5 are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.
III.b.) Ubiquinol Analogs Including a Drug Moiety

In one embodiment, the compound of the invention includes a drug moiety. Exemplary drug moieties are selected from statins, cholesteryl ester transfer protein (CETP) inhibitors (e.g., ezetimibe), bile acid resins (e.g., colestipol, cholestyramine and colesevelam), fibrates (e.g., gemfilbrozil and fenfibrate) and other potentially lipid lowering agents, such as cholesterol acyltransferase (ACAT) inhibitors, ileal bile acid transport (IBAT) inhibitors, microsomal triglyceride transfer protein (MTP) inhibitors, and dual peroxisome proliferator-activated receptor (PPAR) alpha and gamma agonists.

In an exemplary embodiment at least one of Y1 and Y2 in Formula (V) is a drug moiety selected from a statin, such as atorvastatin, fluvastatin, lovastatin, pravastatin, rosuvastatin, and simvastatin. An exemplary compound according to this aspect of the invention includes atorvastatin and has the formula:

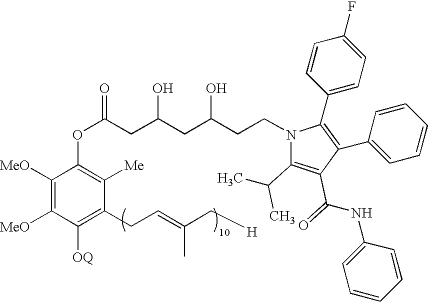

In another embodiment, the drug moiety of Formula (V) is a member selected from nicotinic acid, retinoic acid, azeleic acid, adapalene, salicylic acid, glycolic acid, benzoyl peroxide, erythromycin, neosporin, polysporin, benzalkonium chloride, chlorhexidine, hexachlorophene, fusidic acid, neomycin, oxytetracyclin, mupirocin, flucloxacillin, doxorubicin, acyclovir, tazarotene, olanzapine, and amlodipine.

In yet another exemplary embodiment, the drug moiety of Formula (V) is selected from a drug molecules used for the treatment of a neurological disorder, for example, a neurodegenerative disease, such as Parkinson's disease, Alzheimer's disease, Huntington disease, amyotrophic lateral sclerosis (ALS), and diabetic neuropathy. Exemplary drug molecules according to this aspect of the invention include entacapone, tolcapone, carbidopa, levodopa, benztropine, bromocriptine, donepezil, tacrine, rivastigmine, galantamine, and memantine.

In yet another exemplary embodiment of the invention, the drug moiety of Formula (V) is a member selected from a sleep aid, an anti-depressant, an anxiolytic, and an anti-psychotic. Exemplary drug molecules according to this aspect of the invention include zolpidem, lorazepam, flurazepam, zaleplon, paroxetine, venlafaxine, fluoxetine, buproprion, sertraline, alprazolam, citalopram, methylphenidate, imipramine, amitryptiline, cyamemazine and amoxapine.

In yet another exemplary embodiment of the invention, the drug moiety of Formula (V) is an anti-tumor agent, such as taxol and taxotere.

In another aspect of the invention, both Y1 and Y2 in Formula (V) represent a drug moiety. In an exemplary embodiment, such compound could be used to administer to a patient in need of such treatment a combination of two different drug molecules in the form of a single molecular entity, thereby providing a novel vehicle for combination therapy. In an exemplary embodiment a statin is attached to one of the phenolic hydroxy groups of the reduced ubiquinone in Formula (II), for instance, through an ester linkage. The remaining hydroxy group in the ubiquinol is then used to attach a second lipid lowering drug, such as the CETP inhibitor ezetimibe using the same or another covalent linkage. In vivo, both drugs may be released by hydrolyses thereby setting free three individual components: (i) the LDL lowering statin, (ii) the CETP inhibitor effecting cholesterol absorption, and (iii) CoQ, which may counteract the side effects of statin use. In another exemplary embodiment, a compound according to Formula (V) includes a statin and a drug selected from an Azheimer's treatment, such as memantine.

In an exemplary embodiment of the invention at least one of Y1 and Y2 in Formula A) is dopamine or a dopamine derivative. Exemplary compounds according to this aspect of the invention include: 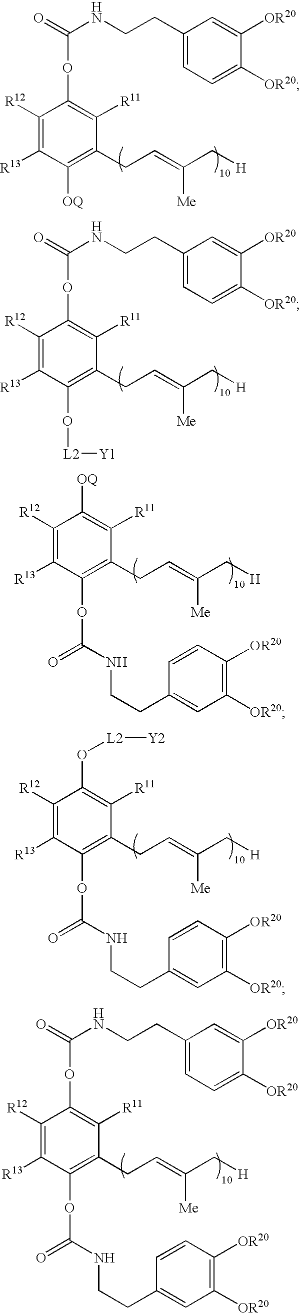
wherein R²⁰ is a member selected from H, substituted or unsubstituted alkyl, substituted or unsubstituted heteroalkyl, substituted or unsubstituted aryl, substituted or unsubstituted heteroaryl and substituted or unsubstituted cycloalkyl and substituted or unsubstituted heterocycloalkyl. Y1, Y2, L2 and Y2 are as defined above. The invention also provides mixtures including at least two of the above molecules.

In an exemplary embodiment R¹¹ is methyl, R¹², R¹³, and R²⁰ are methoxy and the ubiquinol-dopamine adducts have structures according to the following formulae; 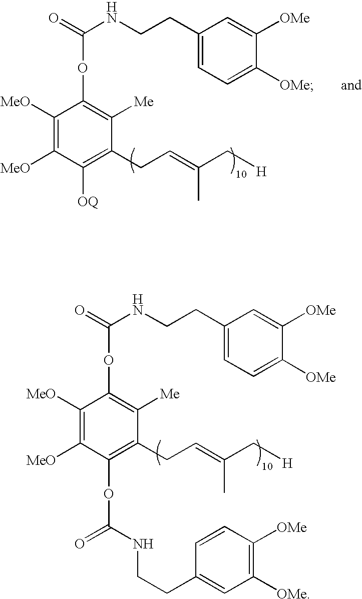
IV. Synthesis of the Compounds

Techniques useful in synthesizing the compounds of the invention are both readily apparent and accessible to those of skill in the relevant art. The discussion below is offered to illustrate certain of the diverse methods available for use in assembling the compounds of the invention and is not intended to define the scope of reactions or reaction sequences that are useful in preparing the compounds of the present invention. The reagents shown in Schemes II to XI, below, are exemplary and can be replaced with other art recognized reagents.

Compounds of the invention may be synthesized according to the synthetic outline, below. The ubiquinone of Formula (I) may be prepared from precursors according to Formulae (III) or (VII) as described herein, below. The ubiquinone can subsequently be reduced to form a ubiquinol analog according to Formula (II). In one embodiment, treatment of the ubiquinol derivative with a base and reaction of the resulting phenolic anion with an electrophilic precursor of a functional moiety (erg., a drug molecule) leads to a compound of the invention.

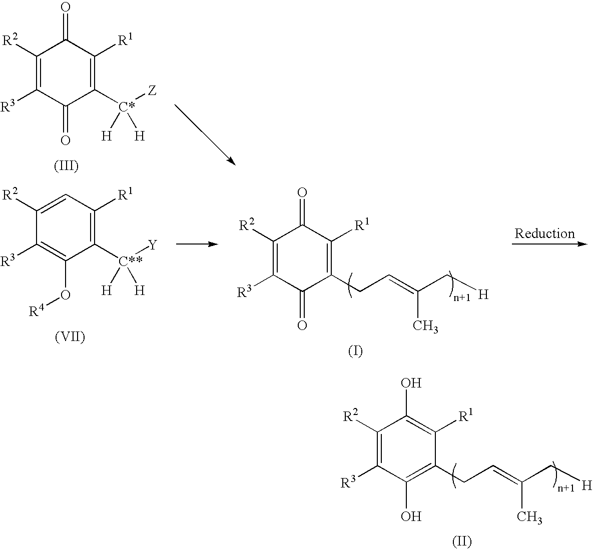

Exemplary ubiquinone and ubiquinol analogs, which can be used as starting materials for the synthesis of the compounds of the invention include: 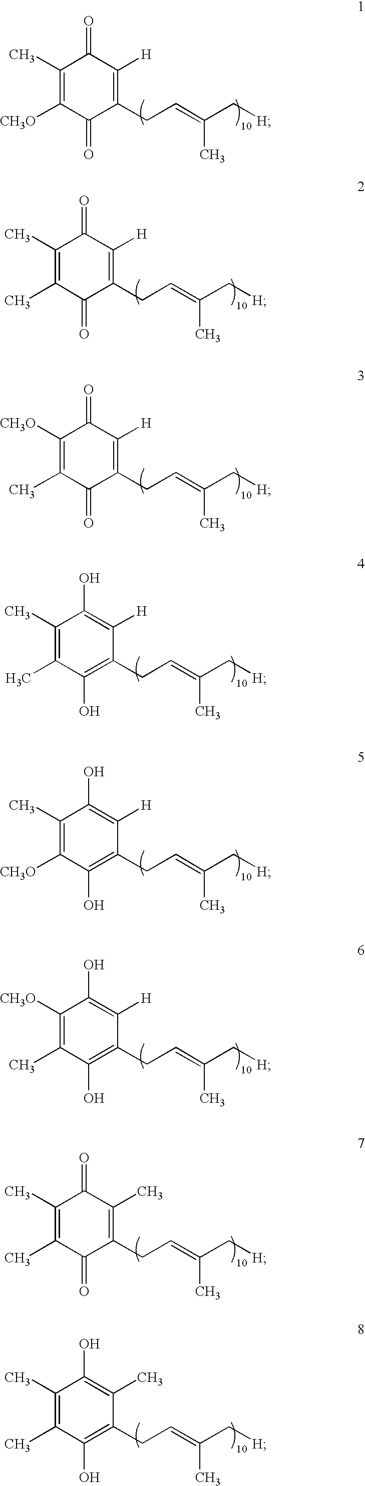 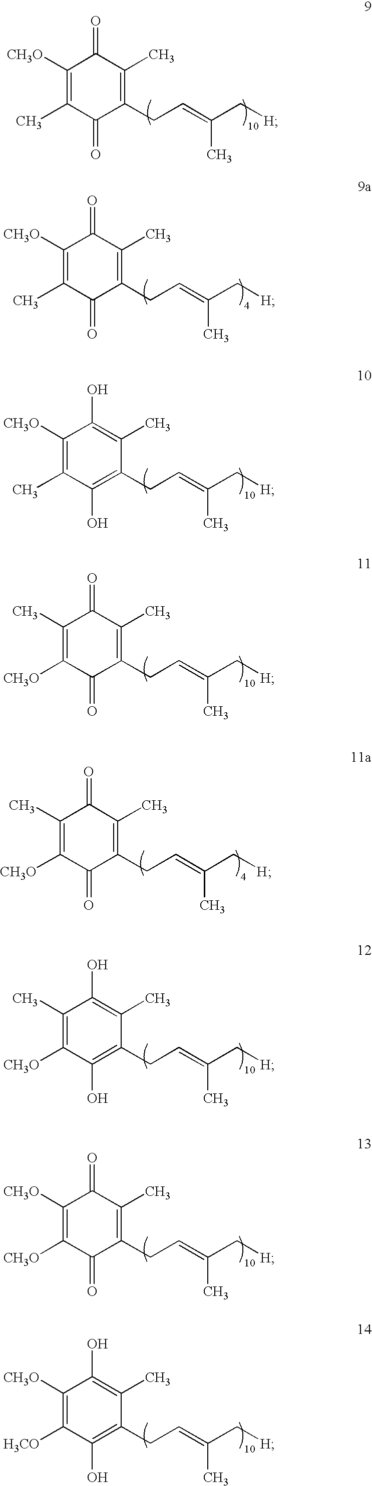 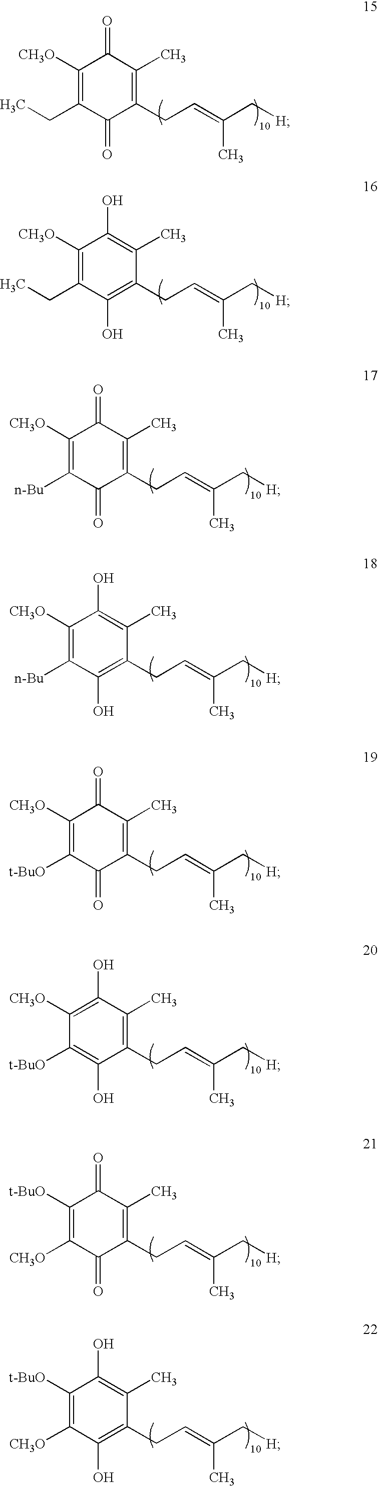 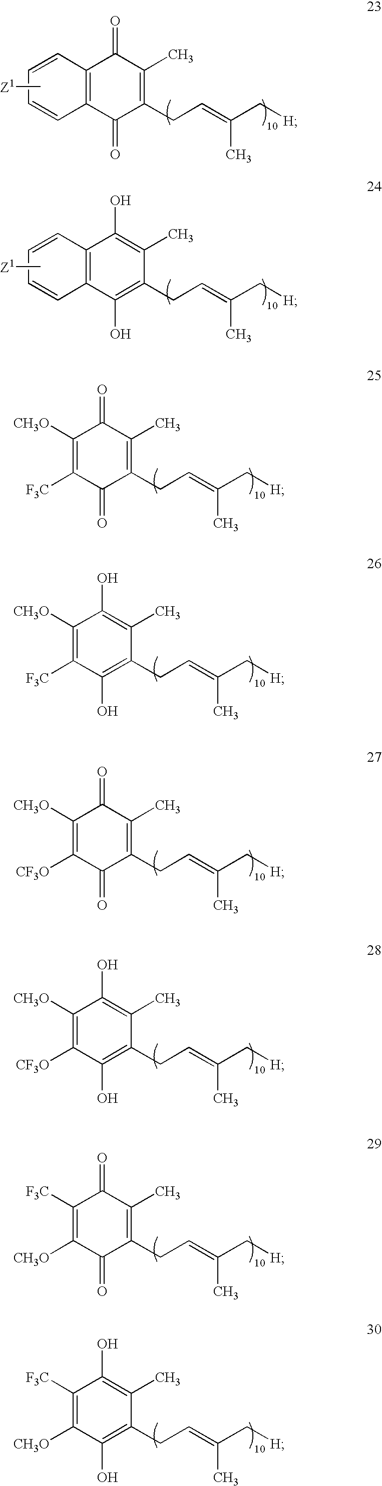 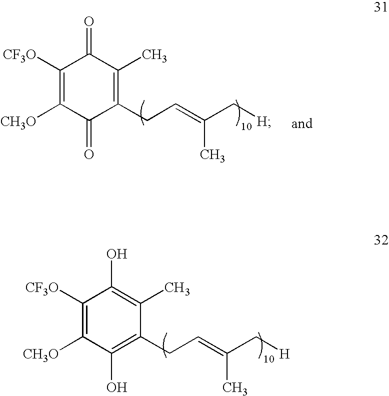
wherein Z¹ is an aryl group substitutent. In an exemplary embodiment, Z¹ is a member selected from H, halogen, CN, substituted or unsubstituted alkoxy and substituted or unsubstituted alkyl.

In another exemplary embodiment, the ubiquinone and ubiquinol analogs have structures according to the following formulae: 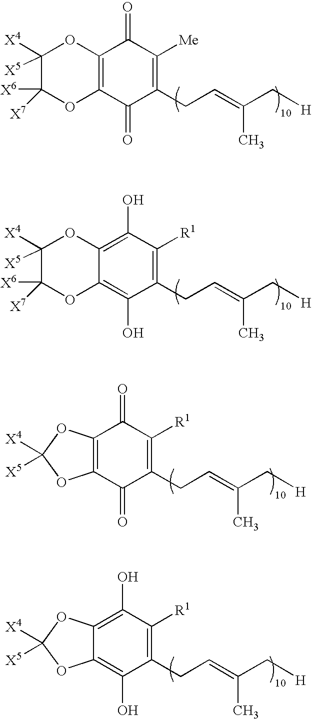
wherein X⁴, X⁵, X⁶ and X⁷ are members independently selected from H and halogen. In an exemplary embodiment the halogen is F.
IV.a.) Synthesis of Compounds According to Formula (III) and Formula (VII)

The ubiquinone precursors according to Formula (III) and the aromatic analogs according to Formula (VII) are prepared by art-recognized methods or modifications thereof. For example, the synthesis of quinones functionalized with a halomethyl group can be accomplished using methods such as those described by Lipshutz et al. (e.g. Tetrahedron 1998, 54: 1241-1253 and J. Am. Chem. Soc. 1999, 121: 11664-11673), the disclosures of which are incorporated herein by reference. In addition, the synthesis of substituted methylene aromatic moieties, such as phenols, can be accomplished using methods described by U.S. Pat. No. 6,545,184 to Lipshutz et al., and U.S. Patent Application No. 20050148675 to Lipshutz et al.; the disclosures of which are also herein incorporated by reference.
IV.a.)i.) Synthesis of the Compounds According to Formula (III)

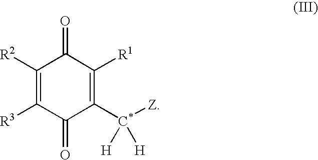

In Formula (III), R¹, R² and R³ are members independently selected from an alkylgroup substitutent. In an exemplary embodiment, R¹, R² and R³ are members independently selected from H, halogen, CN, substituted or unsubstituted alkoxy, substituted or unsubstituted alkyl, substituted or unsubstituted heteroalkyl, substituted or unsubstituted aryl, substituted or unsubstituted heteroaryl and substituted or unsubstituted heterocycloalkyl. R² and R³, together with the carbon atoms which they are attached, are optionally joined to form a 5- to 7-membered ring, which in turn can be part of a fused ring system.

In Formula (III), Z is a leaving group. In a preferred embodiment, Z is a halogen. Currently preferred compounds of Formula (III) include those wherein Z is Cl.

Exemplary compounds of Formula (III), which are useful to prepare the compounds of the invention include:

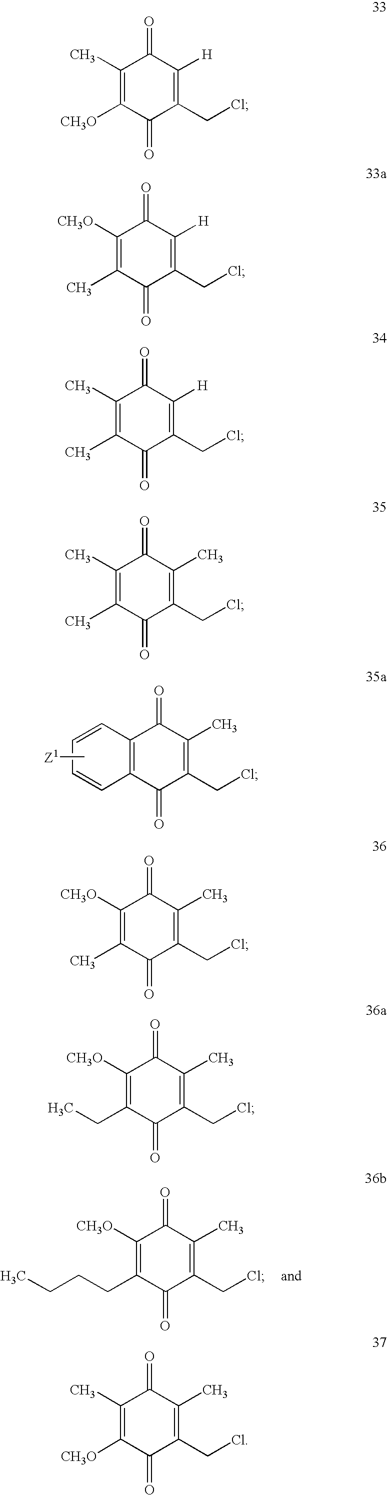

In an exemplary embodiment compounds of Formula (III) are synthesized by performing one of the transformations outlined in Scheme I, below:

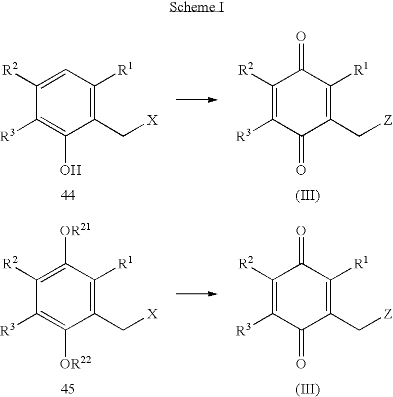

In Scheme I, the substitutents X and Z are independently selected from a leaving group, R⁶, OR⁶, SR⁶ and NR⁶R⁷, wherein R⁶ and R⁷ are members independently selected from H, substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl. R²¹ and R²² are members independently selected from H and lower alkyl. R¹, R² and R³ are as defined above.

In an exemplary embodiment, R¹, R² and R³ are independently selected from H, C₁-C₆ methyl and C₁-C₆ methoxy.

The method includes oxidation of either a phenolic compound 44 or a dialkoxy analog 45 to their corresponding quinones. The method may further include the replacement of the substitutent X with the substitutent Z, either before or after the oxidative step. In an exemplary embodiment X is OH and Z is Cl. In another exemplary embodiment, both X and Z are Cl.

The phenolic intermediate, such as compound 44 can be oxidized directly to the quinone or, alternatively, it can first be converted to the corresponding hydroquinone and then be oxidized to the quinone. An array of reagents and reaction conditions are known that can be used for the oxidation of phenols to quinones, see, for example, Trost B M et al. COMPREHENSIVE ORGANIC SYNTHESIS: OXIDATION, Pergamon Press, 1992.

In an exemplary embodiment, the oxidant includes a transition metal chelate. The chelate is preferably present in the reaction mixture in an amount from about 0.1 mol % to about 10 mol %. In another exemplary embodiment, the transition metal chelate is used in conjunction with an organic base, such as an amine. Exemplary amines are the trialkyl amines, such as triethylamine. In another exemplary embodiment, the transition metal chelate is Co(salen). The chelate can be a heterogeneous or homogeneous oxidant. In an exemplary embodiment, the chelate is a supported reagent. In another exemplary embodiment the oxidizing reagent is ceric ammonium nitrate [Ce(NH₄)₂(NO₃)₆].

The oxidative conversion of a substrate such as compound 44 or 45 to a compound according to Formula (III) is optionally performed under pressure that is greater than ambient pressure. Methods for conducting reactions under pressure are recognized in the art (see, e.g., Matsumoto and Acheson, ORGANIC SYNTHESIS AT HIGH PRESSURE, J. Wiley & Sons, NY, 1991).

In an exemplary embodiment a compound according to Formula (III) is synthesized following the procedure outlined in Scheme II. 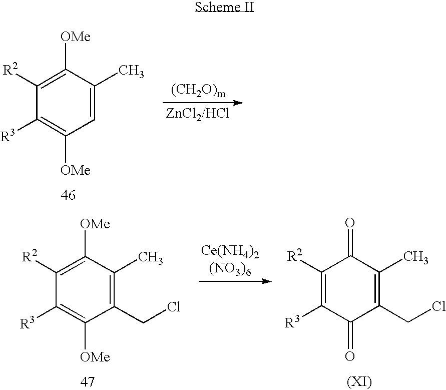
in which R² and R³ are as described above. In an exemplary embodiment according to this embodiment of the invention, R² and R³ are independently selected from substituted or unsubstituted alkyl.

In Scheme II, the dimethyl-protected hydroquinone 46 is converted to a chloromethyl analog 47, which is oxidized to the corresponding chloromethyl quinone (XI). The reagents given in Scheme II are exemplary. Other art recognized reagents can be used to accomplish the shown transformations. For instance, the oxidant ammonium cerium nitrate used in the last step can be replaced with other oxidants described above.

In another exemplary embodiment a compound according to Formula (III) is synthesized following the procedure outlined in Scheme III, below.

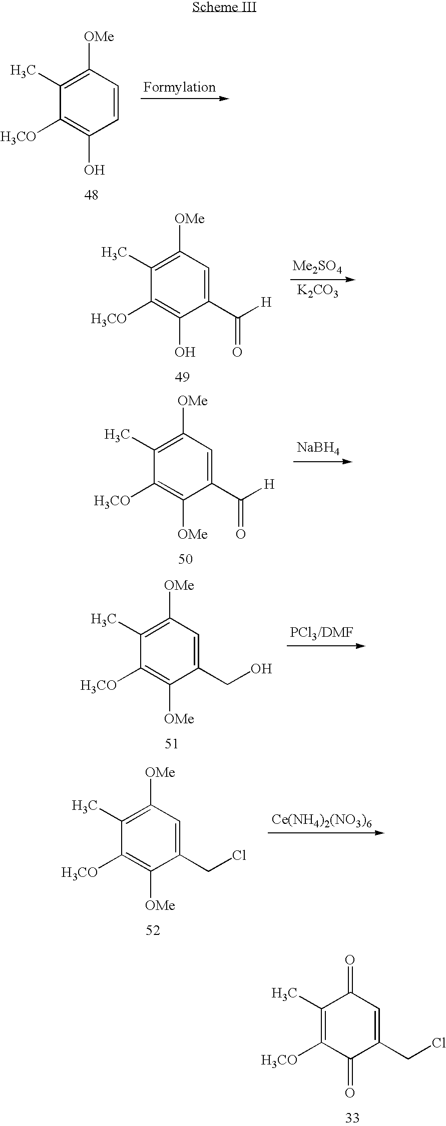

In Scheme III the dimethoxymethylphenol 48 is formylated, yielding the aldehyde 49. After methylation of the phenolic hydroxy group, the aldehyde 50 is reduced to the benzylic alcohol 51, which is further converted to the chloromethyl analog 52. In the final step the chinone 33 is prepared by oxidation.

A wide array of art-recognized formylation methods can be used to introduce an aldehyde function such as that in compound 49. See, for example Jutz et al., Adv. Org. Chem. 1976, 9 (Part 1), 225-342.

A wide array of art-recognized reducing agents can be used for the reduction of an aldehyde, such as 50 to an alcohol, such as 51. See, for example, Trost et al., COMPRETHENSIVE ORGANIC SYNTHESIS: REDUCTION, Pergamon Press, 1992. In an exemplary embodiment, the reducing agent is a reagent that is a source of hydrogen which is a member selected from the group consisting of metal hydrides. In another exemplary embodiment, the reduction is accomplished by catalytic hydrogenation. In yet another exemplary embodiment, the reduction is an electrochemical reduction.

The hydroxyl moiety of 51 is contacted with a halogenating agent, such as thionyl chloride, PCl₃ or another art recognized halogenation reagent affording the halide 52. Alternative halogenation reagents are given in MARCH'S ADVANCED ORGANIC CHEMISTRY, J. Wiley & Sons, NY, 2001).

The reaction pathways set forth in Scheme III can be altered by using a leaving group other than chloro at the methylene group of 52. Examples of useful leaving groups are provided herein. In an exemplary embodiment the hydroxyl moiety of 51 can be converted to an oxygen-containing moiety, such as a benzylic ether, which is prepared by contacting 51 with an alkylating agent. The resulting products can then either be coupled with a reagent according to Formula (IV) in the presence of a catalyst to afford an analog of a compound according to Formula (II), or can be oxidized to afford the corresponding quinone according to Formula (III).

Moreover, the methyl group used to protect the phenolic oxygen atom, for example, in compound 50 can be replaced with a number of art-recognized protecting groups. Useful phenol protecting groups include, but are not limited to, ethers formed between the phenol oxygen atom and substituted or unsubstituted alkyl groups (e.g., methoxy, ethoxy, sulfonic acid esters, methoxymethyl, benzyloxymethyl, methoxyethoxymethyl, 2-(trimethylsilyl)ethoxymethyl, methylthiomethyl, phenylthiomethyl, 2,2-dichloro-1,1-difluoroethyl, tetrahydropyranyl, phenacyl, p-bromophenacyl, cyclopropylmethyl, allyl, isopropyl, cyclohexyl, t-butyl, benzyl, 2,6-dimethylbenzyl, 4-methoxybenzyl, o-nitrobenzyl, 2,6-dichlorobenzyl, 4-(dimethylaminocarbonyl)benzyl, 9-anthrymethyl, 4-picolyl, heptafluoro-p-tolyl, tetrafluoro-4-pyridyl); silyl ethers (e.g., trimethylsilyl, t-butyldimethylsilyl); esters (e.g., acetate, levulinate, pivaloate, benzoate, 9-fluorenecarboxylate); carbonates (e.g., methyl, 2,2,2-trichloroethyl, vinyl, benzyl); phosphinates (e.g., dimethylphosphinyl, dimethylthiophosphinyl); sulfonates (e.g., methanesulfonate, toluenesulfonate, 2-formylbenzenesulfonate), and the like (see, e.g., Greene et at., PROTECTIVE GROUPS IN ORGANIC SYNTHESIS, 3rd ed., John Wiley & Sons, New York, 1999).

In another exemplary embodiment, compounds according to Formula (III) are prepared following the procedure outlined in Scheme IV: 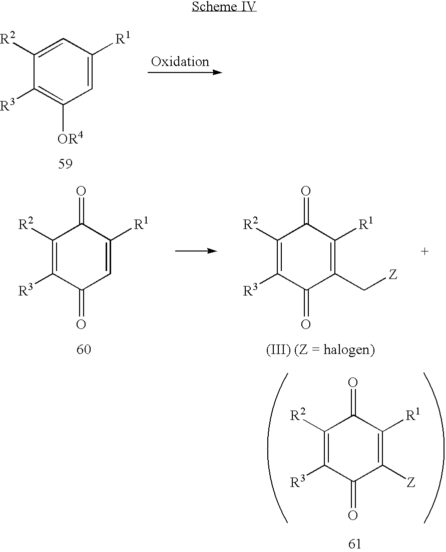
in which R², R³ and R⁴ are as described above. In an exemplary embodiment according to this aspect of the invention, R² and R³ are independently selected from substituted or unsubstituted alkyl, and substituted or unsubstituted alkoxy.

In Scheme IV the quinone 60 is prepared by oxidation of compound 59. The resulting quinone is converted to a compound according to Formula (III), in which Z is a halogen, by the action of formaldehyde in the presence of a selected halohydric acid. The synthetic route in Scheme IV may lead to an undesired side product 61. Methods for the purification of the product mixture can be found in U.S. Pat. No. 6,545,184 to Lipshutz et al., and U.S. Patent Application No. 20050148675 to Lipshutz et al.; the disclosures of which are also herein incorporated by reference.
IV a.) ii.) Synthesis of the Compounds According to Formula (VII)

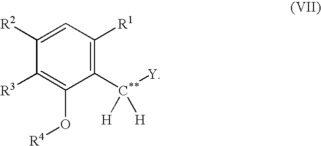

In Formula (VII), R¹, R² and R³ are members independently selected from aryl group substitutents. In an exemplary embodiment, R¹, R² and R³ are members independently selected from H, halogen, CN, substituted or unsubstituted alkoxy, substituted or unsubstituted alkyl, substituted or unsubstituted heteroalkyl, substituted or unsubstituted aryl, substituted or unsubstituted heteroaryl and substituted or unsubstituted heterocycloalkyl. R² and R³, together with the carbon atoms to which they are attached, are optionally joined to form a 5- to 7-membered ring.

In Formula (VII), R⁴ is a member selected from H and a protecting group. R⁴ can be any art-recognized protecting group. Useful phenol protecting groups include, but are not limited to ethers formed between the phenol oxygen atom and substituted or unsubstituted alkyl groups. Examples include: methoxy, ethoxy, sulfonic acid esters, methoxymethyl, benazyloxymethyl, methoxyethoxymethyl, 2-(trimethylsilyl)ethoxymethyl, methylthiomethyl, phenylthiomethyl, 2,2-dichloro-1,1-difluoroethyl, tetrahydropyranyl, phenacyl, p-bromophenacyl, cyclopropylmethyl, allyl, isopropyl, cyclohexyl, t-butyl, benzyl, 2,6-dimethylbenzyl, 4-methoxybenzyl, o-nitrobenzyl, 2,6-dichlorobenzyl, 4-(dimethylaminocarbonyl)benzyl, 9-anthrymethyl, 4-picolyl, heptafluoro-p-tolyl, tetrafluoro-4-pyridyl); silyl ethers (e.g., trimethylsilyl, t-butyldimethylsilyl); esters (e.g., acetate, levulinate, pivaloate, benzoate, 9-fluorenecarboxylate); carbonates (e.g., methyl, 2,2,2-trichloroethyl, vinyl, benzyl); phosphinates (e.g., dimethylphosphinyl, dimethylthiophosphinyl); sulfonates (e.g., methanesulfonate, toluenesulfonate, 2-formylbenzenesulfonate), and the like (see, e.g., Greene et al., PROTECTIVE GROUPS IN ORGANIC SYNTHESIS, 3rd ed., John Wiley & Sons, New York, 1999).

In Formula (VII), Y is a leaving group. In an exemplary embodiment, Y is a halogen. In a preferred embodiment, Y is Cl.

In an exemplary embodiment, a compound according to Formula (VII) is prepared by a method outlined in Scheme V: 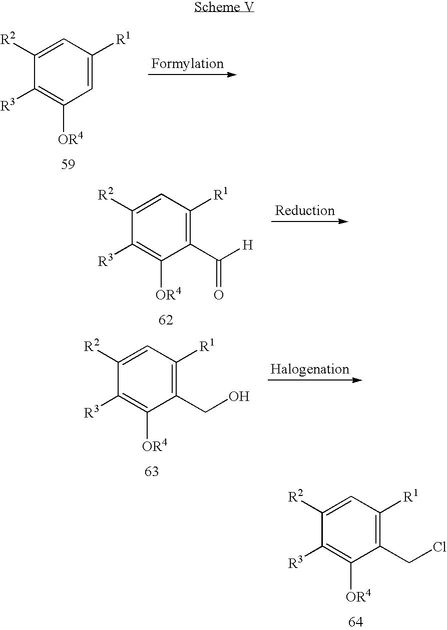
wherein R¹, R², R³ and R⁴ are as defined above.

The method of the invention includes formylating a compound such as 59 to produce an aldehyde such as 62. The aldehyde is contacted with a reducing agent thereby forming an alcohol such as compound 63. The alcohol or the corresponding alkoxide is contacted with a reagent that converts the —OH group into a leaving group, preferrably a halogen such as chloro in compound 64. Alternatively the intermediate formed after contacting compound 62 with the reducing agent is converted directly into the corresponding halide by contacting the intermediate with a protic halide source, such as hydrochloric acid. Formylation, reduction and halogenation methods are described above.

IV.b.) Synthesis of Ubiquinones of Formula (I) and Ubiquinols of Formula (II)

In one aspect, the method of the present invention is based on a retrosynthetic disconnection that relies on the well-known maintenance of olefin geometry in group 10 transition metal coupling reactions (Hegedus, TRANSITION METALS IN THE SYNTHESIS OF COMPLEX ORGANIC MOLECULES, University Science Books, Mill Valley, Calif., 1994). The discussion that follows focuses on a reaction, in which the coupling partners are a vinyl organometallic and a substituted-methylene quinone in which the methylene group is substituted with a leaving group (e.g., halomethyl quinone, ether, sulfonate, etc.). These reactions have similarities to coupling reactions between a vinyl alane and a protected, substituted-methylene phenol, as described in U.S. Pat. No. 6,545,184, the disclosure of which is herein incorporated by reference. The focus of the discussion is for clarity of illustration, and other methods and reagents appropriate for the preparation of the compounds of the invention (e.g., the described coupling reactions) will be apparent to those of skill in the art and are within the scope of the present invention.

IV.b.)i.) Synthesis of Ubiquinone Analogs According to Formula (I)

In an exemplary embodiment, the ubiquinone analogs of Formula (I) are synthesized by contacting a compound having a structure according to Formula (III): 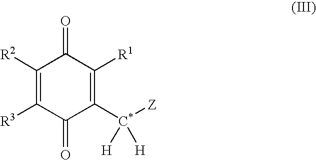
wherein each of R¹, R², R³ and Z is described as above, and an organometallic species having a structure according to Formula (IV).

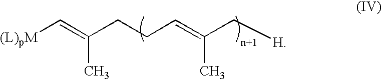

In Formula (IV), n is an integer from 0 to 13. L is an organometallic ligand, M is a metal and p is an integer selected from 1 to 5, wherein each of the p organometallic ligands is independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

The two components are contacted in the presence of a catalyst effective at catalyzing coupling between C* of the substituted methylene group according to Formula (III) and the organometallic species according to Formula (IV), thereby forming the compound according to Formula (I).

The preparation of organometallic species according to Formula (IV) can be accomplished using methods described in U.S. Pat. No. 6,545,184 to Lipshutz et al., and U.S. Patent Application No. 20050148675, the disclosures of which are herein incorporated by reference.

In an exemplary embodiment (L)ₚM- is (L)₂Al—. In a particularly preferred embodiment, (L)ₚM- is (CH₃)₂Al—.

In another aspect, the present invention provides a method of synthesizing the compound according to Formula (I), which includes, contacting a compound having a structure according to Formula (VII): 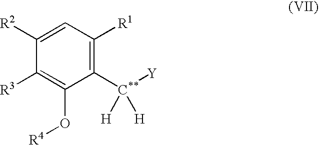
wherein R¹, R², R³, R⁴ and Y are defined as above, and an organometallic species having a structure according to Formula (IV): 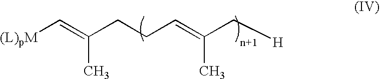
wherein n, L, M and p are as described above.

The two components are contacted in the presence of a catalyst effective at catalyzing coupling between the benzylic carbon atom C** according to Formula (VIII) and said organometallic species according to Formula (IV), thereby forming a compound according to Formula (IX):

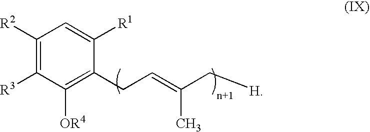

The protecting group R⁴ is then removed to produce a compound according to Formula (X);

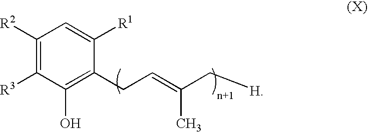

The resulting phenol is oxidized to the quinone of Formula (I) by contacting the compound according to Formula (X) with an oxidant.

In an exemplary embodiment, the leaving group Y in Formula (VII) is a halogen. In a preferred embodiment Y is chloro. In another exemplary embodiment according to this aspect of the invention, (L)ₚM- in Formula (IV) is (L)₂Al—. In a particularly preferred embodiment, (L)ₚM- is (CH₃)₂Al—.

The aromatic precursor according to Formula (VII) can include substantially any useful phenol protecting group R⁴. Preferred R⁴ groups are removed by a reaction that is a member selected from the group consisting of hydrolysis, hydrogenolysis, reduction, oxidation, nucleophilic attack, electrophilic attack and combinations thereof. In an exemplary embodiment R⁴ is —S(O)₂R³⁰. R³⁰ is preferably substituted or unsubstituted alkyl or substituted or unsubstituted aryl, and more preferably p-tolyl. In a still further preferred embodiment, the p-toluenesulfonyl group is removed by contacting the compound with a mixture comprising n-butyllithium, thereby producing the compound according to Formula (X).

IV.b.)i.)A. Organometallic Species of Formula (IV)

The metals, M, of use in the method of the invention include those metals that can carbometallate an alkyne component to produce a species according to Formula (IV). In an exemplary embodiment, metals include transition metals and aluminum. The metal can be formally neutral or it can be charged (e.g. an aluminate). The transition metal chemistry can be catalytic or stoichiometric. For example, the alkyne can be metalated by catalytic carbocupration using Cu(I) to form an adduct that is subsequently transmetalated to the corresponding zinc reagent.

The coordination number of M is satisfied by the bonding or coordination to the metal ion of the requisite number of organometallic ligands, such as Lewis base donors (e.g., halogen donors, oxygen donors, mercaptide ligands, nitrogen donors, phosphorous donors, and heteroaryl groups); hydrides; carbon ligands bound principally by σ-bonds (e.g., alkyls, aryls, vinyls, acyl and related ligands); carbon ligands bound by σ- and π-bonds (e.g., carbonyl complexes, thiocarbonyl, selenocarbonyl, tellurocarbonyl, carbenes, carbynes, σ-bonded aetylides, cyanide complexes, and isocyanide complexes); ligands bound through more than one atom (e.g., olefin complexes, ketone complexes, acetylene complexes, arene complexes, cyclopentadienyl complexes, π-allyl complexes); unsaturated nitrogen ligands (e.g., macrocyclic imines, dinitrogen complexes, nitric oxide complexes, diazonium complexes); and dioxygen complexes. Other useful combinations of metal ions and ligands will be apparent to those of skill in the art. See, for example, Collman J P et al. PRINCIPLES AND APPLICATIONS OF ORGANOTRANSITION METAL CHEMISTRY, University Science Books, 1987.

In an exemplary embodiment, the organometallic species according to Formula (IV) is a carboaluminated species. In one embodiment the carboaluminated species 54 is synthesized following the procedure outlined in Scheme VI: 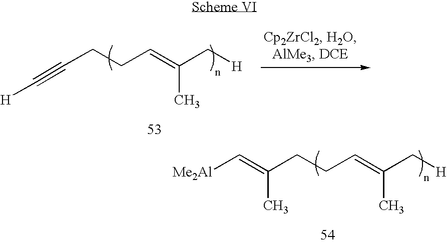
in which n is as defined as above.

Method for the preparation of the carboaluminated species with an alkyl moiety bound to aluminum according to Formula (IV), including compound 54, are described in U.S. Pat. No. 6,545,184 to Lipshutz et at, and U.S. Patent Application No. 20050148675 to Lipshutz et al.; the disclosures of which are herein incorporated by reference.

In one aspect, the method of carboalumination utilizes a metal species, e.g., a zirconium or titanium complex, in a catalytic quantity, which means in an amount of less than 1 molar equivalent relative to the alkyne substrate 53. Catalysts for this reaction are referred to herein as “carboalumination catalysts”. For example, the catalyst can be present in amounts of 0.1 to 20 mole %, preferably from about 0.5 to about 5.0 mole % relative to the alkyne. It has been discovered that minimizing the amount of zirconium species present does not have a deleterious effect on the efficiency of the carboalumination. Thus, the invention provides a method of carboalumination, using a catalytic amount of a metal species, e.g., a zirconium or titanium species that provides the carboaluminated species in high yields.

An exemplary carboalumination catalyst of use in the present invention is Cp₂ZrCl₂. Those of skill in the art will recognize that numerous other metal-based catalysts, such as titanocenes and zirconocenes, are of use as carboalumination catalysts in the invention.

In this embodiment, the invention is based on recognition that the remaining organometallic carboalumination catalyst (e.g., the zirconium salts), rather than the potential organic impurities, is problematic in the coupling of carboaluminated alkyne (IV) and a quinone (e.g., 1 or 3) to form a compound of Formula (I) or Formula (II), and that minimization of the carboalumination catalyst allows for a shortened (“one pot”) route to the target ubiquinone. Thus, when a minimized amount of a zirconium or titanium species is used (e.g. ≦10 mole %), the carboaluminated product does not have to be separated prior to its being used in a coupling reaction with a quinone. Surprisingly, no marked degradation in the purity or quantity of the coupling product results from omitting the purification step.

In another exemplary embodiment, the carboalumination process is conducted in the presence of substoichiometric amounts of water, an alcohol (R^{x}OH) or methylaluminoxane (MAO), and in the presence of about 0.5 to 20 mole % of a coupling catalyst (e.g. a zirconium or titanium species as described above). Preferably the subsequent coupling reaction is carried out without prior removal of the carboalumination catalyst or the species derived thereof from the resulting vinyl alane. This allows conducting the carboalumination and the subsequent coupling as a “one pot” reaction, i.e. a reaction that is conducted in one vessel. The present methodology offers a convenient access to ubiquinone and ubiquinol analogs and offers the advantage of applicability to a technical scale.

The carboalumination reaction can yield mixtures of regioisomeric vinyl alanes, which in turn lead to mixtures of ubiquinone regioisomers in the subsequent cross coupling reaction with the C* methylene carbon of chloromethylated quinones according to Formula III. The factors influencing the regioselectivity of the carboalumination are well known to those skilled in the art. Those include for example the temperature, the nature of the solvent and of the carboalumination catalyst.

IV.b.)i.)B. Cross Coupling of Organometallic Species with Compounds of Formula (III)

In one aspect of the invention, compounds according to Formula (I) are synthesized following the general procedure outlined in Scheme VII.

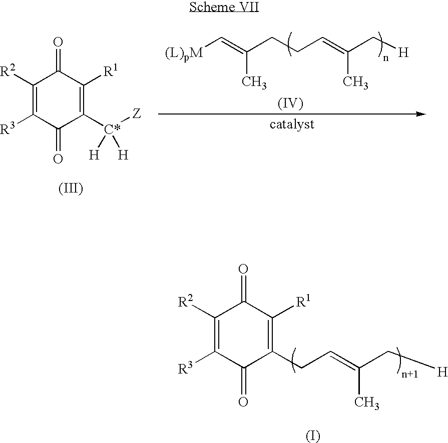

In Scheme VII a substituted-methylene moiety according to Formula (III), wherein Z is a leaving group, and in which the substitutents R¹, R², and R³ are as discussed above, is contacted with a carboaluminated species having a structure according to Formula (IV), wherein M, L, p and n are defined as above.

The coupling of compound (III) with a compound according to Formula (IV) affords the compound according to Formula (I). The two components are contacted in the presence of a coupling catalyst that is effective at catalyzing coupling between C of the substituted methylene group in Formula (III), and the vinylic carbon attached to the metal on the compound according to Formula (IV).

IV.b.)i.)C. Cross Coupling Catalysts

In an exemplary embodiment, the coupling catalyst utilizes a species that includes a transition metal. Exemplary transition metal species of use as coupling catalysts include, but are not limited to, those metals in Groups IX, X, and XI. Exemplary metals within those Groups include Cu(I), Pd(0), Co(0) and Ni(0). Recent reports have demonstrated that catalyst couplings, using the appropriate reaction partners and based on metal catalysis, are quite general and can be used to directly afford known precursors (Naruta, J. Org. Chem., 45:4097 (1980); Eren, et al., J. Am. Chem. Soc., 110:4356 (1988) and references therein; Van Lient et al., Rec. Trav Chim. Pays-Bays 113:153 (1994); Rüttiman et al., Helv. Chim. Acta, 73;790 (1990); Terao et al., J. Chem. Soc., Perkin Trans. 1:1101 (197), Lipshutz et al., J. Am. Chem., Soc. 121: 11664-11673 (1999); Lipshutz et al., J. Am. Chem. Soc. 118: 5512-5313 (1999)). In another exemplary embodiment, the metal is Ni(0).

The coupling catalyst can be formed by any of a variety of methods recognized in the art. In an exemplary embodiment in which the transition metal is Ni(0), the coupling catalyst is formed by contacting a Ni(II) compound with two equivalents of a reducing agent, reducing Ni(II) to Ni(0). In an exemplary embodiment, the Ni(II) compound is NiCl₂(PPh₃)₂. In yet another exemplary embodiment, the reducing agent is n-butyllithium. In yet another exemplary embodiment, the method of the invention includes contacting NiCl₂(PPh₃)₂, or a similar Ni species, with about two equivalents of a reducing agent (e.g., n-butyllithium), thereby reducing said NiCl₂(PPh₃)₂ to Ni(0). Alternatively, other readily available forms of Ni(0) can be employed (e.g., Ni(COD)₂).

The coupling catalyst can be a homogeneous or heterogeneous catalyst (Cornils B, Herrmann W A, APPLIED HOMOGENEOUS CATALYSIS WITH ORGANOMETALLIC COMPOUNDS: A COMPREHENSIVE HANDBOOK IN TWO VOLUMES, John Wiley and Sons, 1996; Clark J H, CATALYSIS OF ORGANIC REACTIONS BY SUPPORTED INORGANIC REAGENTS, VCH Publishers, 1994; Stiles AB, CATALYST SUPPORTS AND SUPPORTED CATALYSTS: THEORETICAL AND APPLIED CONCEPTS, Butterworth-Heinemann, 1987). In one exemplary embodiment, the coupling catalyst is supported on a solid material (e.g., charcoal, silica, etc.). In another exemplary embodiment, the coupling catalyst is a supported nickel catalyst (see, e.g., Lipshutz et al., Synthesis, 2110 (2002); Lipshutz et al., Tetrahedron 56:2139-2144 (2000); Lipshutz and Blomgren, J. Am. Chem. Soc. 121: 5819-5820 (1999); and Lipshutz et al., Inorganica Chimica Acta 296: 164-169 (1999).

The method of the invention is practiced with any useful amount of coupling catalyst effective at catalyzing coupling between the methylene carbon atom on the aromatic group or of the quinone moiety mentioned above, and the vinylic carbon attached to M on the compound according to Formula (IV). In an exemplary embodiment, the coupling catalyst is present in an amount from about 0.1 mole % to about 10 mole %. In an exemplary embodiment, the coupling catalyst is present in an amount from about 0.5 mole % to about 5 mole %. In an exemplary embodiment, the coupling catalyst is present in an amount from about 2 mole % to about 5 mole %.

The above mentioned coupling reaction can be carried out in all solvents known to those of skill in the art, suitable as solvents for transition metal catalyzed coupling reactions, e.g., ethers, such as THF, diethyl ether and dioxane; amines, e.g., triethylamine, pyridine and NMI; as well as other solvents, such as acetonitrile, acetone, ethyl acetate, DMA, DMSO, NMP and DMF. In a preferred embodiment, it is not required to completely remove the solvent in which the carboalumination was carried out, prior to the coupling.

The conditions of the coupling reaction can be varied. For example, the order of addition of reactants can be varied. In an exemplary embodiment, the substituted methylene moiety and carboaluminated species are contacted, and then the coupling catalyst is subsequently added. In an exemplary embodiment, the substituted methylene moiety and coupling catalyst are contacted, and then the carboaluminated species is subsequently added. In an exemplary embodiment, the coupling catalyst and carboaluminated species are contacted, and then the substituted methylene moiety is subsequently added.

The amount of the substituted methylene moiety relative to the alkyne employed in the prior carboalumination can also be varied. In an exemplary embodiment, the substituted methylene moiety, can be reacted in amounts ranging from 0.9 to 10 equivalents relative to the alkyne mentioned above. In another exemplary embodiment, the substituted methylene moiety can be reacted in amounts ranging from 0.9 to 5 equivalents, preferably from 0.9 to 2, and most preferably from 1.1 to 1.6 equivalents, relative to the alkyne mentioned above.

The coupling reaction of the present invention can be conducted under a variety of conditions. For example, the coupling reaction can be conducted at a temperature from —40° C. to 50° C. In an exemplary embodiment, the temperature of the coupling reaction can be room temperature. In another exemplary embodiment, the temperature of the carboalumination reaction can be from −30° C. to 0° C. In another exemplary embodiment, the temperature of the carboalumination reaction can be from about −25° C. to about −15° C.

The length of time for the coupling reaction can vary from 10 minutes to 10 hours. Typically, the lower the temperature at which the reaction is conducted, the longer the amount of time it takes for the reaction to go to completion. When the temperature is about 0° C., the reaction can be completed from about 30 minutes to about 3 hours.

A representative example for preparing a ubiquinone of Formula (I), starting with quinone 35 is set forth in Scheme VIII.

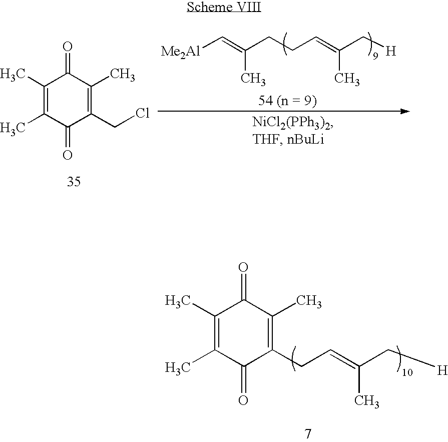

In Scheme VIII, the chloromethyl quinone 35 is contacted with the vinylalane 54 in the presence of a Ni(O) catalyst. The reaction affords the corresponding ubiquinone 7.

IV.b.)ii) Synthesis of Ubiquinol Analogs According to Formula (II)

In another aspect the present invention provides a method of making a compound of Formula (II), which includes, synthesizing a compound according to Formula (I) by one of the above methods, and reducing the intermediate, thereby forming a compound according to Formula (II). The procedure is outlined in Scheme IX, below.

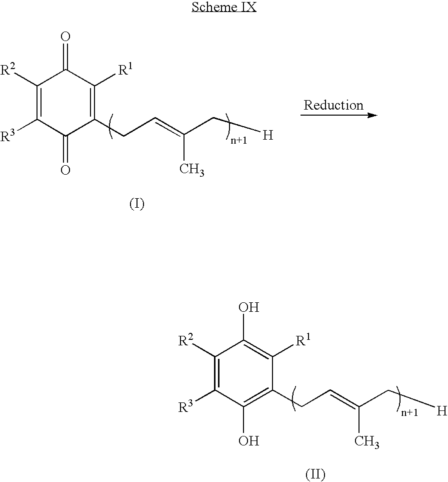

In Scheme IX a ubiquinon analog according to Formula (I) is reduced to afford a hydroquinone according to Formula (II), in which the substitutents R¹, R², and R³ and the integer n are as discussed above.

Reducing agents that can be used for this conversion are known in the art and include Zn reagents, peroxidisulfates (S₂O₈²⁻), and Sn reagents (e.g. SnCl₂). Alternatively the reduction can be accomplished using catalyic hydrogenation.

In another aspect of the invention compounds according to Formula (II) are synthesized following the procedure outlined in Scheme X: 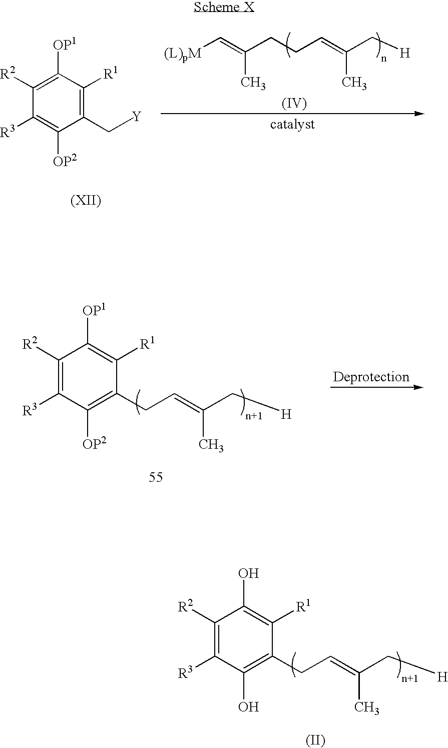
wherein Y is a leaving group and n, R¹, R², and R³ are as discussed as above for Formula (VII). P¹ and P² are protecting groups.

In an exemplary embodiment Y is a halogen, such as chloro.

In another exemplary embodiment the protecting groups P¹ and P² include ethers formed between the phenol oxygen atom and substituted or unsubstituted alkyl groups (e.g. methoxy and ethoxy groups). Other useful protecting groups include sulfonic acid esters, methoxymethyl, benzyloxymethyl, methoxyethoxymethyl, 2-(trimethylsilyl)ethoxymethyl, methylthiomethyl, phenylthiomethyl, 2,2-dichloro-1,1-difluoroethyl, tetrahydropyranyl, phenacyl, p-bromophenacyl, cyclopropylmethyl, allyl, isopropyl, cyclohexyl, t-butyl, benzyl, 2,6-dimethylbenzyl, 4-methoxybenzyl, o-nitrobenzyl, 2,6-dichlorobenzyl, 4-(dimethylaminocarbonyl)benzyl, 9-anthrymethyl, 4-picolyl, heptafluoro-p-tolyl, tetrafluoro-4-pyridyl); silyl ethers (e.g., trimethylsilyl, t-butyldimethylsilyl); esters (e.g., acetate, levulinate, pivaloate, benzoate, 9-fluorenecarboxylate); carbonates (e.g., methyl, 2,2,2-trichloroethyl, vinyl, benzyl); phosphinates (e.g., dimethylphosphinyl, dimethylthiophosphinyl); sulfonates (e.g., methanesulfonate, toluenesulfonate, 2-formylbenzenesulfonate), and the like (see, e.g., Greene et al., PROTECTIVE GROUPS IN ORGANIC SYNTESiS, 3rd ed., John Wiley & Sons, New York, 1999). In an exemplary embodiment P¹ and P² are members independently selected from CH(O) and substituted or unsubstituted alkyl.

In an exemplary embodiment, P1 and P2 do not include a hydrophilic polymeric moiety selected from a polyether and a polyalcohol.

P1 and P2 are preferrably removed by a reaction that is a member selected from hydrolysis, hydrogenolysis, reduction, oxidation, nucleophilic attack, electrophilic attack and combinations thereof. In an exemplary embodiment at least one of the protecting groups P1 and P2 is —S(O)₂R³⁰ wherein R³⁰ is preferably substituted or unsubstituted alkyl or substituted or unsubstituted aryl, and more preferably p-tolyl. In a still further preferred embodiment, the p-toluenesulfonyl group is removed by contacting the compound with a mixture comprising n-butyllithium, thereby producing the compound according to Formula (II).

In Scheme X the Y-substituted-methylene moiety according to Formula (XII), is contacted with a carboaluminated species having a structure according to Formula (IV), wherein M, L, p, and n are as defined above to afford compound 55. The coupling conditions are similar to those described above for the synthesis of the ubiquinones according to Formula (I). In the last step the protecting groups are removed to afford the compound of Formula (II). Alternatively only one of the protecting groups can be removed, while the other remains intact.

In an exemplary embodiment, P¹ is a CH(O) moiety and P² is methyl, as show in Scheme XI.

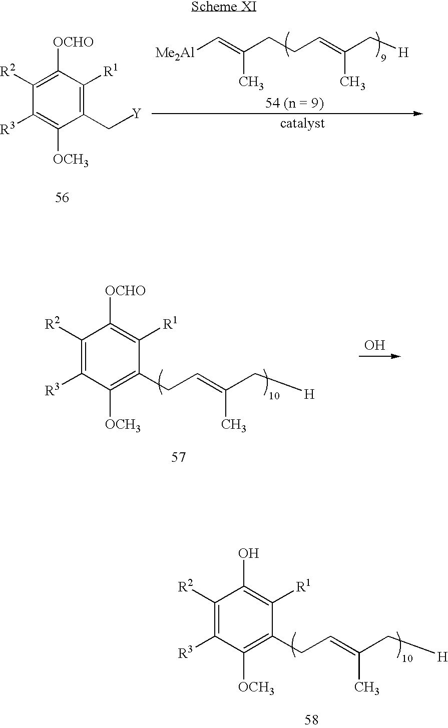

In Scheme XI the OCH(O) moiety of compound 56 is a protecting group that remains intact during the alkylation to produce compound 57. The CH(O) group is removed by hydrolytic cleavage to afford the resulting phenol 58, which can alternatively be oxidized to the corresponding ubiquinone.

Methods for the purification of the ubiquinones and ubiquinoles of the invention, are provided in U.S. Pat. No. 6,545,184 to Lipshutz et at, and U.S. Patent Application No, 20050148675 to Lipshutz et at, the disclosures of which are herein incorporated by reference.

The synthetic schemes set forth herein are intended to be exemplary of the synthesis of compounds of the invention. Those of skill in the art will recognize that many other synthetic strategies leading to compounds within the scope of the present invention are available. For example, by modification of the starting materials a compound having an ethoxy, rather than a methoxy group can be produced. Moreover, leaving and protecting groups discussed herein can be replaced with other useful groups having a similar function.

V. Pharmaceutical Compositions

In a further aspect, the present invention provides pharmaceutical compositions comprising a compound of the invention (i.e., a compound of Formula (V)) or a pharmaceutically acceptable salt thereof and a pharmaceutically acceptable carrier.

The compounds of the present invention can be prepared and administered in a wide variety of oral, parenteral and topical dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 700 of the active modulator. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active modulator with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

VI. Pharmaceutical Methods

In another aspect the invention provides a method of treating a neurological disorder. The method includes administering to a subject in need of such treatment, a pharmaceutically effective amount of a compound of the invention or a pharmaceutically acceptable salt or solvate thereof. In a preferred embodiment, the subject is a human.

In an exemplary embodiment, the neurological disorder is a member selected from central nervous system ischemia, central nervous system trauma, epilepsy, seizures, neurodegenerative diseases, vascular dementia, manic-depressive psychosis, depression, schizophrenia, chronic pain (e.g., neuropathic pain), trigeminal neuralgia, migraine, ataxia, bipolar disorder, spasticity, mood disorders, psychotic disorders, hearing and vision loss, age-related memory loss, learning deficiencies, anxiety and cerebral edema.

In another exemplary embodiment, the central nervous system trauma is a member selected from brain trauma, spinal cord injury and whiplash injury. In yet another exemplary embodiment, said neurodegenerative disease is a member selected from amyotrophic lateral sclerosis (ALS), Alzheimer's disease, Huntington disease, Parkinson's disease and diabetic neuropathy. In a further embodiment, the vascular dementia is a member selected from multi-infarct dementia and Binswanger's disease.

In one embodiment of the invention, a compound according to Formula (V) can be used to facilitate the delivery of a drug moiety to the central or peripheral nervous system. In an exemplary embodiment, the ubiquinol analog of the invention is used to enable the drug moiety to cross the blood brain barrier.

Accordingly, in another aspect, the invention provides a method of increasing the concentration of a drug molecule in the tissue of the brain or another organ of the central or peripheral nervous system. The method includes administering to a subject in need of such treatment, a therapeutically effective amount of a compound of the invention (i.e., a compound of Formula (V) or a pharmaceutically acceptable salt or solvate thereof), wherein the compound includes the drug molecule. The increase of drug concentration in the tissue (e.g., of the brain) is referenced against a reference concentration, which results from administration of a comparable amount of “unbound” drug molecule through the same route of administration. “Unbound” in this context means that the drug molecule is not part of a molecule of the invention (i.e., is not bound to a ubiquinol moiety).

In an exemplary embodiment, the increase in drug concentration is measured using a suitable animal model and measuring the drug concentrations resulting from both the administration of drug molecule, which is part of a compound of the invention and administration of a comparable amount of drug, which is not bound to a ubiquinol moiety. Drug concentrations may be obtained through mass spectral analysis of samples prepared from a suitable tissue sample. In another embodiment, the increase is manifested through a therapeutic activity, which is not observed, or not observed to the same extent, when administering a comparable amount of drug, wherein the drug is not part of a compound of the invention.

Hence, in another aspect, the invention provides a method of enhancing the therapeutic activity of a drug molecule. The method includes providing a compound of the invention which includes the drug molecule and administering a therapeutically effective amount of the compound (i.e., a compound of Formula (V) or a pharmaceutically acceptable salt or solvate thereof) to a subject in need of such treatment. In this context “enhancing” can also mean “having” or “showing” a therapeutic activity, which is not otherwise observed to with the drug molecule alone (unbound drug molecule), when administered in a comparable amount and through the same route of administration. Therapeutic activity can be any biological effect. In one embodiment, therapeutic activity is measured using a suitable animal model of disease (e.g., an animal model of a neurological disorder, such as Parkinson's disease). For example, a first group of rodents is treated with a compound of is the invention that includes the drug molecule. A control group of rodents is treated (through the same route of administration) with a comparable amount of the drug molecule, with the difference that the drug molecule is not bound to a ubiquinol moiety (unbound drug molecule). A biological effect (e.g., shrinkage of a tumor, severity of depression-like symptoms, Parkinson's-like symptoms and the like) is measured in the first group and in the control group and results are compared. In an exemplary embodiment, therapeutic activity is enhanced when the first group shows improved symptoms as compared to the control group

In another embodiment of the invention a compound of Formula (V) can be used for the systemic delivery of lipid lowering drugs. In an exemplary embodiment, administration of the drug using a molecule of the invention may positively affect the pharmaceutical properties of the drug molecule and/or may reduce drug interactions. In another exemplary embodiment, use of the current compounds for drug delivery may reduce pharmaceutically effective dosages. In yet another exemplary embodiment, the use of these compounds may reduce adverse events, which are normally associated with the drug when administered in a comparable amount and through the same route of administration.

In a further embodiment, compounds according to Formula (V), which include both a drug moiety and a targeting moiety can be used to direct the drug moiety to a particular organ or tissue type. Such approach may reduce the pharmaceutically effective dose and potential side effects normally associated with administration of the drug.

In a further aspect the invention provides a method of lowering the cholesterol concentration in the bloodstream of a patient. The method includes administering to a subject in need of such treatment, a pharmaceutically effective amount of a compound of the invention or a pharmaceutically acceptable salt or solvate thereof, wherein the compound includes at least one drug moiety selected from a CETP inhibitor and a statin.

In yet another aspect, the invention provides a method of lowering the concentration of low-density lipoprotein (LDL) in the bloodstream of a patient. The method includes administering to a subject in need of such treatment, a pharmaceutically effective amount of a compound of the invention or a pharmaceutically acceptable salt or solvate thereof, wherein the compound includes at least one drug moiety that is a statin.

The materials, methods and devices of the present invention are further illustrated by the examples that follow. These examples are offered to illustrate, but not to limit the claimed invention.

### EXAMPLES

General

In the examples below, unless otherwise stated, temperatures are given in degrees Celsius (° C.); operations were carried out at room or ambient temperature, “rt,” or “RT,” (typically a range of from about 18-25° C.); evaporation of solvent was carried out using a rotary evaporator under reduced pressure (typically, 4.5-30 mm Hg) with a bath temperature of up to 60° C.; the course of reactions was typically followed by thin layer chromatography (TLC) and reaction times are provided for illustration only; melting points are uncorrected; products exhibited satisfactory ¹H-NMR and/or microanalytical data; yields are provided for illustration only; and the following conventional abbreviations are also used, mp (melting point), L (liter(s)), mL (milliliters), mmol (millimoles), g (grams), mg (milligrams), min (minutes), h (hours), RBF (round bottom flask).

The following chemicals were subjected to the following preparatory steps prior to use in the Examples. PCl₃ was refluxed for 3 h at 76° C. while slowly purging with dry argon to expel HCl, distilled at atmospheric pressure and stored in a sealed container under argon until needed. DMF, 2-propanol and benzene were used as supplied from Fisher chemicals. Solanesol, purified by column chromatography on SiO₂ with 10% diethyl ether/petroleum ether, was dried azeotropically with toluene or benzene immediately prior to use. TI-IF was distilled from Na/benzophenone ketyl prior to use. n-BuLi was obtained as a 2.5 M solution in hexanes from Aldrich and standardized by titration immediately prior to use. Ethanol was 200 proof, dehydrated, U.S.P. Punctilious grade. All other reagents were purchased from suppliers and used without further purification. Products were confirmed by ¹H NMR, ¹³C NMR, IR, LREIMS and HR-EI or HR-Cl Mass Spectrometry. TLC and chromatographic solvents are abbreviated as follows: EA: ethyl acetate; PE: petroleum ether; DCM: dichloromethane.

### Example 1

### Synthesis of Compound 1

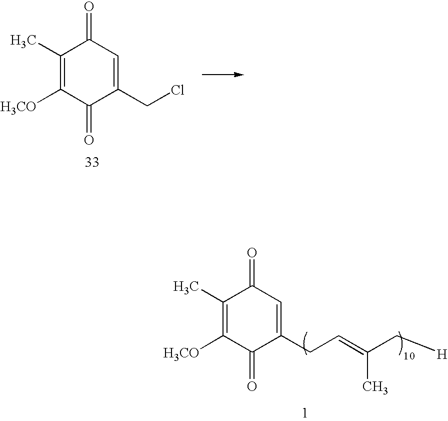

The synthesis of compound 1 consists of four distinct portions; the synthesis of the chloromethylated analog 33, carbalumination of an alkyne to afford the alane 54 (n=9), preparation of the Ni(0) coupling catalyst, and cross coupling of the alane 54 to 33.

### 1.1 Synthesis of 5-(chloromethyl)-3-methoxy-2-methylcyclohexa-2,5-diene-1,4-dione 33

### 1.1.a 1-(2,4-Dimethoxy-3-methylphenyl)ethanone

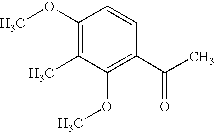

In a dry, argon-flushed, round bottom flask equipped with a stir bar, acetyl chloride (0.09 mL, 0.11 g, 1.32 mmol) was added to TiCl₄ (0.15 mL, 0.25 g 1.32 mmol) at −10° C. and stirred for 0.1 h. Once the flask cooled to −10° C., a solution of 2,6-dimethoxytoluene (0.18 g, 0.96 mmol) in distilled benzene (5 mL) was added over the course of 10 min with vigorous stirring. During the addition, the temperature was maintained at 0° C. The solution was stirred at 0° C. for 0.5 h, at which point it was complete. The reaction mixture was subsequently poured into a slurry of ice and HCl (1M, 5 mL), extracted with ether (3×5 mL), washed with aqueous NaHCO₃ and brine, dried over Na₂SO₄, and concentrated down to afford a pale, yellow oil (0.14 g, 95%). The crude product was used in the subsequent step without further purification. Full spectral data for this compound has been reported (Knolker, H-J.; Frohner, W.; Reddy, K. R.,; Synthesis 2002, 4, 557-564).

### 1.1.b 2,4-Dimethoxy-3-methylphenylacetate

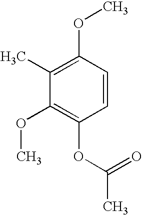

In a dry, argon-flushed round bottom flask, recrystallized mCPBA (2.43 g, 14.0 mmol) was dissolved in distilled CH₂Cl₂ (15 mL). In a second, identical flask, the acetophenone (2.78 g, 11.74 mmol) and TsOH (0.10 g, 0.6 mmol) was dissolved in CH₂Cl₂ (5 mL) and cooled to 0° C. With vigorous stirring, the contents of the first flask were slowly transferred via cannula to the second flask. The resulting suspension was allowed to warm to rt and stirred for 4 h. When the reaction was complete according to TLC analysis (Rf=0.4, 10% ethyl acetate:hexanes), Na₂SO₃ (1M, 20 mL) was added and stirred for 0.25 h to destroy any excess peracid. Additionally, saturated aqueous NaHCO₃ (20 mL) was added and the mixture was stirred for 0.25 h to neutralize. The organics were extracted with CH₂Cl₂ (3×30 mL), washed with brine, dried over Na₂SO₄, and concentrated down to a red-orange oil. Distillation of the crude product afforded a light-yellow oil (2.2 g, 90%) that solidified in the refrigerator. Full spectral data for this compound has been reported (Knolker, H-J.; Frohner, W.; Reddy, K. R.; Synthesis 2002, 4, 557-564).

### 1.1.c 2,4-Dimethoxy-3-methylphenol

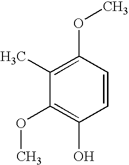

In a round bottom flask equipped with a stir bar, aryl acetate (5.2 g, 24.8 mmol) was dissolved in ethanol (20 mL) and stirred at rt. Over a period of 10 min, a solution of KOH (6.9 g, 123.8 mmol) in water (10 mL) was added and an orange-brown solution formed. The solution was stirred for 2-3 h, at which point TLC analysis (Rf=0.45, 33% ether:hexanes) showed the reaction had gone to completion, and the ethanol was evaporated in vacuo. The aqueous layer was acidified to pH=4 using HCl (6M) and the organics were extracted with ether (3×50 mL), washed with brine, and dried over Na₂SO₄. Removal of the solvent and distillation in vacuo provided a clear oil (3.7 g, 88%) that solidified in the refrigerator. Full spectral data for this compound has been reported (Knolker, H-J.; Frohner, W.; Reddy, K. R.; Synthesis 2002, 4, 557-564).

### 1.1.d 2-Hydroxy-3,5-dimethoxy-4-methylbenzaldehyde

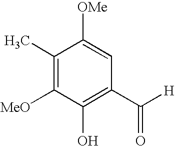

In a dry, argon-flushed round bottom flask, fresh trifluoroacetic acid and trifluoroacetic anhydride (1:1, 10 mL each) were mixed and cooled to 0° C. In a second identical flask, phenol (5.0 g, 30.0 mmol) and hexamethylenamine (6.25 g, 45.0 mmol) were weighed out, purged under an argon atmosphere, and cooled to 0° C. The pre-mixed TFA: TFAA solution was then slowly transferred to the second flask via cannula with vigorous stirring. Once the initial exotherm subsided, the reaction was warmed to rt. A water-jacket condenser was then attached to the flask and the mixture was heated to reflux for 45 h. The initially orange mixture turned red-black. When TLC analysis (Rf=0.3, 30% ether: hexanes) showed the reaction had gone to completion, the mixture was poured slowly into a flask containing a slurry of ice and aqu. NaHCO₃. Once the mixture was neutralized, the organics were extracted with ether (3×50 mL), washed with brine, dried over Na₂SO₄, and concentrated down to a red-black solid. The solid was taken up in ether and pushed through a plug of silica to remove polymeric by-products. The organics were then concentrated down to afford light yellow needle-shaped crystals (4.6 g, 78%) taken on without further purification to the next step.

### 1.1.e 2,3,5-Trimethoxy-4-methylbenzaldehyde

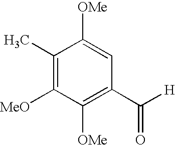

In a dry, argon-flushed round bottom flask equipped with a stir bar, phenol (5.4 g, 27.4 mmol) and K₂CO₃ (11.4 g, 82.3 mmol) were added to acetone (30 mL) and stirred at 0° C., Me₂SO₄ (4.1 g, 3.1 mL, 32.9 mmol) was added to the reaction dropwise at 0° C. until the exotherm subsided, then the mixture was warmed to room temperature and heated to 40° C. for 8 h. Upon completion (TLC Rf=0.6, 30% ether:hexanes), the reaction was neutralized with HCl (IM) and the organics were extracted with ethyl acetate (3×50 mL), washed with brine, dried over Na₂SO₄, and evaporated down to a brown oil. Flash column chromatography on silica gel with a solution of 25% ether:hexanes as eluant afforded a white, crystalline solid (5.26 g, 91%).

HRMS Calcd for C₁₁H₁₄O₄: 210.0884. Found: 210.0892. Ms m/z (%): 210(100), 195(60), 180(6), 167(12), 152(10), 137(11), 124(9), 109(12), 53(19). IR (KBr) cm⁻¹: 2939, 2856, 1682, 1599, 1470, 1407, 1386, 1333, 1134, 739, 629. ¹H NMR (CDCl₃) δ, 10.351 (s, 1H), 7.036(s, 1H), 3.954(s, 3H), 3.862(s, 3H), 3.846(s, 3H), 2.20(s, 3H). ¹³C NMR (CDCl₃)δ: 189.7, 154.7, 152.3, 151.6, 130.0, 127.2, 102.3, 62.8, 60.7, 56.0, 9.9.

### 1.1.f (2,3,5-trimethoxy-4-methylphenyl)methanol

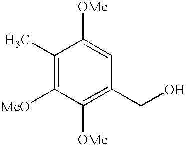

In a round bottom flask equipped with a stir bar, aldehyde was dissolved in THF (5 mL) and cooled to 0° C. NaBH₄ (0.33 g, 8.94 mmol) was added and the reaction was allowed to warm to rt. The yellow solution turned clear as the aldehyde was reduced, After 4 h, no starting material was visible by TLC (product Rf=0.4, 35% ethyl acetate:hexanes). The flask was cooled back down to 0° C. and acidified to pH=2 using HCl (6M). The resulting alcohol was extracted with ethyl acetate (3×30 mL), washed with brine, dried over Na₂SO₄, and concentrated down to a clear oil (0.60 g, 96%) The crude product was carried on without further purification.

### 1.1.g 1-(Chloromethyl)-2,3,5-trimethoxy-4-methylbenzene

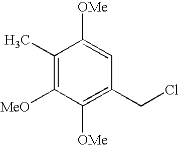

In a dry, argon-flushed round bottom flask equipped with a stir bar, freshly distilled DMF (0.65 g, 0.70 mL, 8.94 mmol) and freshly distilled PCl₃ (1.22 g, 0.78 mL, 8.94 mmol) were combined and stirred at 0° C. for 0.5 h until the two liquids had formed a white, crystalline solid (Vilsmeier's salt). In a second identical flask, the benzylic alcohol (0.64 g, 3.0 mmol) was dissolved in anhydrous THF (5 mL) and transferred to the first flask via cannula. The reaction was then allowed to warm to rt and stirred 2 h, after which no starting material remained according to TLC(Rf=0.8, 30% ethyl acetate:hexanes). A slurry of ice and sat. aqu. NaHCO₃ was prepared and the reaction mixture was carefully poured into the slurry in order to quench any remaining Vilsmeier reagent. The quench was stirred for 1 h. Once the reaction mixture was neutralized, the organics were extracted using ether (3×30 mL), washed with brine, dried with Na₂SO₄, and concentrated down to a yellow solid. The crude was pushed through a silica plug and concentrated down to afford a white solid (0.54 g of 96% pure material, 78%) taken on to the next step without further purification.

### 1.1.h 5-(chloromethyl)-3-methoxy-2-methylcyclohexa-2,5-diene-1,4-dione 33

In a round bottom flask covered in foil and equipped with a stir bar, ceric ammonium nitrate (6.31 g, 11.92 mmol) was dissolved in a solution of CH₃CN:H₂O (10:1, 100 mL) and stirred vigorously. Benzylic chloride (0.538 g, 2.33 mmol) dissolved in a minimal amount of CH₃CN was then added to the solution of CAN and allowed to stir at room temperature for 2-4 h until the reaction appeared to be complete by TLC(Rf=0.6, 20% ethyl acetate:hexanes). Ether (25 mL) and water (25 mL) were then added to the flask to create two distinct layers and the organics were extracted using ether (3×30 mL), washed with brine, dried over Na₂SO₄, and concentrated down to a bright orange-red solid. Purification using flash column chromatography on silica gel with a gradient eluant starting with 100% hexanes and ending at 25% ethyl acetate:hexanes afforded the chloromethylquinone 33 as a yellow-orange oil (0.33 g, 70%). Care was taken to run the column quickly, in the absence of light, in order to limit decomposition. The purified chloromethylquinone 33 was then stored under an inert argon atmosphere at −78° C. until further use.

HRMS Calcd for C₉H₉O₃Cl: 200.0247. Found: 200.0240. Ms m/z(%): 200(15), 164(100), 157(15), 134(40), 109(20), 83(22), 67(50), 49(22). IR (KBr) cm⁻¹: 2949, 1657, 1610, 17, 1379, 1292, 1204, 1226, 1009, 935, 872, 737, 673, 490. ¹NMR (CDCl₃) δ: 6.822(s, 1H), 4.394(d, J=1.6, 2H), 4.022(s, 3H), 1.961(s, 3H). ¹³C NMR (CDCl₃) δ: 8.76, 39.08, 61.05, 129.53, 133.79, 141.86, 155.80, 182.80, 187.71.

### 1.2 Synthesis of the Alane 54 (Carboalumination)

To a dry, argon purged 25 mL round bottom flask was added Cp₂ZrCl₂ (0.011 g, 0.05 mmol) and DCE (0.5 mL). The flask was sealed under argon and cooled to 0° C. Me₃Al (0.76 mL, 1.5 mmol) was added dropwise, followed by MAO (10% w/w solution in toluene, 44 μL, 0.10 mmol) and the solution was allowed to age 0.5 h. Solanesol alkyne (0.727 g, 90% purity, 0.654 g, 11.0 mmol) dissolved in DCE (0.5 mL) was added to the reaction flask at 0° C. and the pale yellow solution was allowed to warm to rt. After 4-8 h, the solution turned a bright amber-yellow color and the alkyne was entirely consumed according to TLC (product Rf=0.9, 5% ether:p.ether) to afford the alane 54 (n=9). The solvent was then removed in vacuo, replaced with distilled THF (1.0 mL), and the flask was cooled to −20° C.

### 1.3 Preparation of the Ni(0) Catalyst

In a separate flask, n-BuLi (2.7M solution, 30 μL, 0.08 mmol) was added to a solution of NiCl₂(PPh₃)₂ (0.027 g, 0.04 mmol) in THF (0.5 mL). The initially gray, heterogeneous solution of Ni(II) turned red-black upon reduction. This slurry was immediately transferred to the flask containing the vinyl alane from step 1.2.

### 1.4 Cross Coupling of Alane 54 with Compound 33

In another flask, 5-(chloromethyl)-3-methoxy-2-methylcyclohexa-2,5-diene-1,4-dione (0.10 g, 0.50 mmol) was dissolved in THF (0.5 mL), cooled to 0° C., and added to the flask containing the vinyl alane. The coupling was allowed to stir at −20° C. for 1-2 h without exposure to light, at which point the reaction had gone to completion. At this stage, two different methods could be employed to work up and isolate the product. In the first method, the reaction was diluted with ether and poured over a plug of anhydrous Na₂SO₄ and silica. The bright yellow organics were concentrated in vacuo to a crude orange-brown oil, dissolved in pet. ether (20 mL) and added to another round bottom flask containing a heterogeneous mixture of zinc dust (0.065 g, 1.0 mmol) in AcOH: H₂O (0.5 mL: 10 mL). This was stirred at rt for 4 h allowing the quinone to reduce to the hydroquinone. Once the reduction was complete according to TLC(R_{f}=0.3, 5% ethyl acetate:hexanes) the ether layer containing the hydroquinone was transferred via cannula to an argon-flushed separatory funnel. The product washed several times with degassed sat. aq. NaHCO₃ to remove any residual acetic acid. Concentration in vacuo and purification using flash column chromatography on silica gel under argon in a gradient, degassed solvent system of 0-25% ethyl acetate:hexanes afforded the pure reduced product as a clear oil (0.105 g, 23% (2 steps)). The oil was then dissolved in CH₃CN: H₂O (15 mL: 5 mL) and CAN (0.27 g, 0.5 mmol) was added at rt. The orange solution was allowed to stir for 1 h without exposure to light, at which point the oxidation was complete. Purification using flash column chromatography on silica gel with 10% ethyl acetate:hexanes afforded compound 1 as a bright, orange-yellow oil (0.084 g, 19% (3 steps)). During purification, care was taken to avoid exposure of the product to light.

The second simpler and higher yielding method involved warming the reaction to rt once the coupling was complete. After several hours of stirring at rt, the quinone was fully reduced in situ to the hydroquinone. The entire reaction mixture was then diluted with ether and poured over a plug of silica. The silica was rinsed several times more with ether, and the organics were concentrated in vacuo to afford a yellow oil. Purification using flash column chromatography on silica gel under argon in a gradient, degassed solvent system of 0-25% ethyl acetate:hexanes afforded the pure reduced product as a clear oil (0.214 g, 49%). This was then subjected to the same oxidation described in the first method. Purification using flash column chromatography on silica gel with 10% ethyl acetate: hexanes afforded compound 1 as a bright orange-yellow oil (0.17 g, 39% (3 steps), R_{f}=0.75, 10% ethyl acetate:hexanes). It should be noted that the R_{f} of the oxidized final product is virtually identical to the R_{f} of the quenched vinyl alane byproduct, making direct isolation of the coupled product difficult. The final product was stored under an argon atmosphere at −78° C. in the absence of light to minimize decomposition.

HRMS Calcd for C₅₈H₈₈O₃: 832.6701. Found: 832.6733. Ms m/z (%): 835(110), 684(8), 688(31), 410(76), 327(31), 259(50), 161(6), 135(14), 109 (19). IR Kr) cm⁻¹: 2961, 2922, 2852, 1655, 1610, 1446, 1377, 1298, 1196, 1128, 1008, 843. ¹H NMR (CDCl₃) δ: 6.437 (s, 1H), 5.153-5.105 (m, 10H), 3.994 (s, 3H), 3.110 (d, J=7.2 Hz, 2H), 2.087-1.942 (m, 39H), 1.688 (s, 6H), 1.632-1.582 (m, 27H). ¹³C NMR (CDCl₃) δ: 188.82, 183.76, 156.20, 146.66, 140.18, 135.66, 135.28, 135.12, 132.29, 131.44, 129.06, 125.23, 124.59, 124.44, 123.95, 118.01, 60.99, 40.22, 39.94, 32.18, 27.18, 26.89, 26.65, 26.91, 23.64, 17.89, 16.35, 16.21, 8.78. E_{pc} values (mV) vs. Ag/AgNO₃ reference electrode: −1038, −1350. Regioisomer ratio: about 88:12, about 6% hydroquinone impurity.

### Example 2

### Synthesis of Compound 2

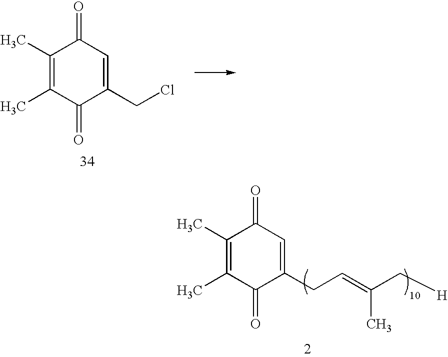

### 2.1. Synthesis of 5-chloromethyl)-2,3-dimethylcyclohexa-2,5-diene-1,4-dione 34

### 2.1.a 1,4-Dimethoxy-2,3-dimethylbenzene

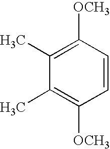

In a round bottom flask equipped with a stir bar, hydroquinone (3.1 g, 22.3 mmol) was dissolved in ethanol (95%, 15 mL) and cooled to 0° C. A solution of Me₂SO₄ (5.7 mL, 6.3 g, 66.9 mmol) and aqueous NaOH (2.4 g, 60.0 mmol in 10 mL H₂O) was then added dropwise to the flask of hydroquinone, turning the solution dark red. Once the exotherm resided, the solution was allowed to warm to rt and stirred an additional 24 h, until the reaction was complete according to TLC analysis (product Rf=0.75, 20% ethyl acetate:hexanes). The solution was acidified to pH=3 with HCl (6M). The organics were extracted with ethyl acetate (3×30 mL), washed with brine, dried with Na₂SO₄, and concentrated down to a red oil. Flash column chromatography on silica gel in a solution of 10% ethyl acetate:hexanes afforded the methoxy- protected hydroquinone as a white crystalline solid (3.37 g, 90%). R_{f}=0.4, 15% ethyl acetate:hexanes). Full spectral data for this compound has been reported previously (Lipshutz, B. H.; Kim, S-k.; Mollard, P.; Stevens, K. L. Tetrahedron, 1998, 54, 1241-1253).

### 2.1.b 1-(chloromethyl)-2,5-dimethoxy-3,4-dimethylbenzene

In a round bottom flask, methoxy- protected hydroquinone (1.10 g, 6.62 mmol) was dissolved in conc. HCl and stirred at rt. ZnCl₂ (0.90 g, 6.62 mmol) and paraformaldehyde (0.27 g, 7.9 mmol) were then added and stirred at rt. After 2 h, water was added to the flask and the organics were extracted with ether (3×30 mL), dried over Na₂SO₄, and concentrated down to a green-brown solid. Flash column chromatography on silica gel in a solution of 2% ether:hexanes afforded an off-white solid that contained a 4:1 mixture of the inseparable mono- and dichloromethylquinone (1.12 g, 91%). This mixture was taken on to the next step without further purification.

### 2.1.c 5-(chloromethyl)-2,3-dimethylcyclohexa-2,5-diene-1,4-dione (34)

In a 200 mL round bottom flask covered with foil and equipped with a stir bar, ceric ammonium nitrate (13.0 g, 28.0 mmol) was dissolved in a solution of CH₃CN: H₂O (50 mL: 2.5 mL) and stirred at rt. To this was added benzylic chloride (1.5 g, 7.0 mmol). The solution was allowed to stir at rt for 4 h, at which point the no starting material remained according to TLC (product Rf=0.6, 20% ether:hexanes), Ether (50 mL) and water (50 mL) were added to the flask to separate the aqueous and organic layers. The product was extracted with ether (3×20 mL), washed with brine, dried over Na₂SO₄, and concentrated down to a red oil. Flash column chromatography on silica gel in a solution of 2% ether:hexanes as eluant afforded the chloromethylquinone 34 as a yellow-orange oil that solidified in the refrigerator (0.52 g, 40%). Care was taken to minimize exposure to light in order to limit decomposition.

### 2.2, Synthesis of Compound 2

Compound 2 was prepared from the chloromethyl analog 34 and the alane 54 (n=9) following the cross coupling procedures outlined in Example 1. The following reagents were used in the specified amounts: 5-(chloromethyl)-2,3-dimethylcyclohexa-2,5-diene-1,4-dione 34 (0.10 g, 0.50 mmol), solanesyl propyne (0.489 g, 0.75 mmol), Cp₂Cl₂ (0.011 g, 0.05 mmol), AlMe₃ (0.56 mL, 2 M solution in toluene, 1.12 mmol), MAO (0.025 mL, 0.0022 g, 0.04 mmol), NiCl₂(PPh₃ (0.027 g, 0.04 mmol), THF (2 mL), nBuLi (30 FL, 2.5 M solution in hexanes, 0.08 mmol). Flash column chromatography on silica gel in 2% ether: hexanes afforded 2 as a yellow solid (0.26 g, 59% (two steps), R_{f}=0.75, 13% ether: hexanes).

HRMS Calcd for C₅₈H₈₈O₂: 816.6757. Found: 816.6784. Ms m/z (%): 816(5), 190(14), 188(100), 135(10), 93(18), 81(37), 69(55). IR (KBr) cm⁻¹ 2964, 2922, 2853, 1649, 1616, 1448, 1383, 1317, 1101, 910, 843, 735, 650. ¹H NMR (CDCl₃) δ: 6.474 (t, J=1.6, 1H), 5.166-5.082 (m, 10H), 3.127 (d, J=7.2 Hz, 2H), 2.106-1.970 (m, 42H), 1.687 (s, 6H), 1.630-1.579 (m, 27H). ¹³C NMR (CDCl₃) δ187.98, 187.84, 148.11, 141.13, 140.73, 139.87, 135.58, 135.09, 132.18, 131.41, 124.59, 124.44, 123.98, 118.28, 39.93, 27.65, 26.88, 26.64, 25.90, 17.88, 16.32, 16.21, 12.6, 12.4. E_{pc} values (mV) vs. Ag/AgNO₃ reference electrode: −1211, −1357. Regioisomeric ratio: 92:8, 15% hydroquinone impurity.

### Example 3

### Synthesis of Compound 7

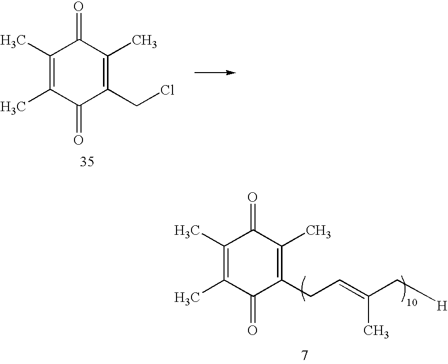

### 3.1. Synthesis of 5-(chloromethyl)-2,3,5-trimethylcyclohexa-2,5-diene-1,4-dione 35

### 3.1.a 1,4-dimethoxy-2,3,5-trimethylbenzene

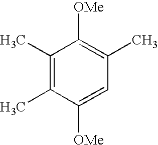

In a round bottom flask equipped with a stir bar, hydroquinone (2.0 g, 13.2 mmol) was dissolved in ethanol (95%, 15 mL) and cooled to 0° C. A solution of Me₂SO₄ (3.4 mL, 3.7 g, 39.5 mmol) and aqu. NaOH (2.4 g, 60.0 mmol in 10 mL H₂O) was then added dropwise to the flask of hydroquinone, turning the solution dark red. Once the exotherm resided, the solution was allowed to warm to rt and stirred an additional 2-4 h, until the reaction was complete according to TLC analysis (product Rf=0.75, 20% ethyl acetate; hexanes). The solution was acidified to pH=3 with HCl (6M). The organics were extracted with ethyl acetate (3×30 mL), washed with brine, dried with Na₂SO₄, and concentrated down to a red oil. Flash column chromatography on silica gel in a solution of 10% ethyl acetate:hexanes afforded the methoxy- protected hydroquinone as a white crystalline solid (2.1 g, 88%). Full spectral data for this compound has been reported (Lipshutz, B. H.; Kim, S-k.; Mollard, P.; Stevens, K. L. Tetrahedron, 1998, 54, 1241-1253).

### 3.1.b 1-(Chloromethyl)-2,4-, dimethoxy-3,4,6-trimethylbenzene

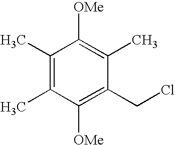

In a round bottom flask, methoxy- protected hydroquinone (1.30 g, 7.2 mmol) was dissolved in conc. HCl and stirred at rt. ZnCl₂ (11 g, 8.0 mmol) and paraformaldehyde (0.50 g, 14.4 mmol) were then added and stirred at rt. After 2 h, water was added to the flask and the organics were extracted with ether (3×30 mL), dried over Na₂SO₄, and concentrated down to a green-brown solid. Flash column chromatography on silica gel in a solution of 2% ether:hexanes afforded an off-white solid that contained a 4:1 mixture of the inseparable mono- and dichloromethylquinone (1.50 g, 90%). This mixture was taken on to the next step without further purification. Full spectral data for this compound has been reported (Lipshutz, B. H.; Kim, S-k.; Mollard, P.; Stevens, K. L. Tetrahedron, 1998, 54, 1241-1253).

### 3.1.c 5-(Chloromethyl)-2,3,5-trimethylcyclohexa-2,5-diene-1,4-dione 35

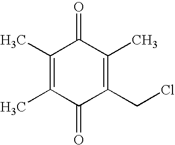

In a 200 mL round bottom flask covered with foil and equipped with a stir bar, ceric ammonium nitrate (9.29 g, 20.0 mmol) was dissolved in a solution of CH₃CN: H₂O (50 mL: to 2.5 mL) and stirred at rt. To this was added benzylic chloride (1.15 g, 5.0 mmol). The solution was allowed to stir at rt for 4 h, at which point no starting material remained according to TLC (product Rf=0.6, 20% ether:hexanes). Ether (50 mL) and water (50 mL) were added to the flask to separate the aqueous and organic layers. The product was extracted with ether (3×20 mL), washed with brine, dried over Na₂SO₄, and concentrated down to a red oil. Flash column chromatography on silica gel in a solution of 2% ether: hexanes as eluant afforded the chloromethylquinone 35 as a yellow-orange oil that solidified in the refrigerator (0.90 g, 78%). Full spectral data for this compound has been reported (Lipshutz, B. H.; Kim, S-k.; Mollard, P.; Stevens, K. L. Tetrahedron, 1998, 54, 1241-1253).

### 3.2. Cross Coupling of the Alane 54 (n=9) and Compound 35

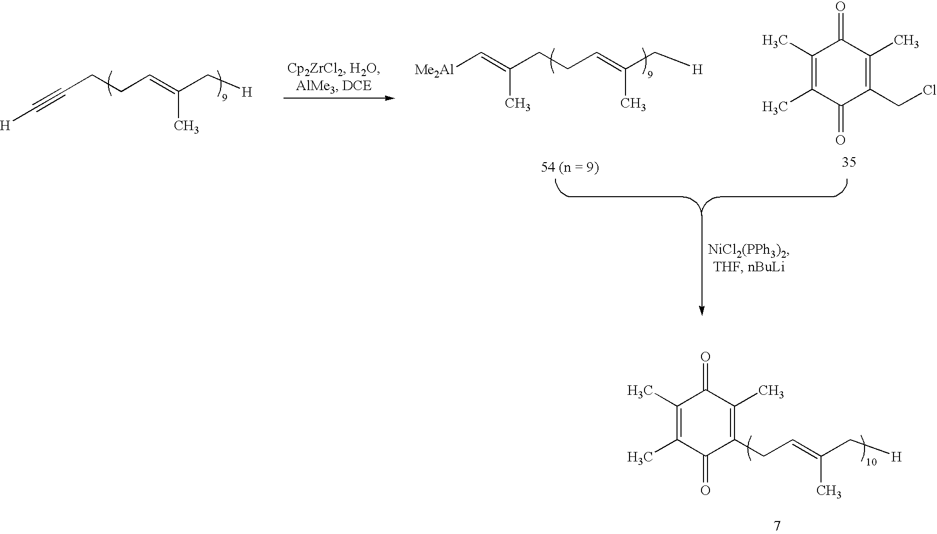

Compound 7 was synthesized using the cross coupling procedures outlined in Example 1+The amounts of reagents were as follows: solanesyl propyne (0.700 g, 1.1 mmol), Cp₂ZrCl₂ (16.1 mg, 0.055 mmol), DCE (2 mL), H₂O (005 FL), NiCl(PPh₃)₂ (35 mg, 0055 mmol), THF (3.0 mL), nBuLi (26.2 μL, 0.11 mmol), compound 35 (109.15 mg, 0.55 mmol). Flash chromatography using silica gel treated with a very small amount of Et₃N (0.25%) and a gradient hexanes; Et₂O solvent system (100% hexanes to 95%:5% hexanes:Et₂O) yielded 352 mg of an orange oil (77.0%).

HRMS Calcd for C₅₉H₉₀O₂: 830.6923. Found: 830.6940. Ms m/z (%): 831(10), 286(3), 243(3), 219(8), 203(94), 165(23), 69(100). 1R (KBr) cm⁻¹: 2964, 2922, 2853, 1649, 1616, 1448, 1383, 1317, 1101, 910, 842, 735, 650. ¹H NMR (CDCl₃) δ: 5.10 (m, 9H), 4.96 (t, J=1.2 Hz, 1H), 3.20 (d, J=7.2 Hz, 2H), 2.10-1.93 (m, 42H), 1.75 (s, 3H), 1.68 (s, 3H), 1.64-1.56 (m, 30H), ¹³C NMR (CDCl₃) δ: 187.9, 187.0, 143.2, 140.4, 140.3, 137.1, 135.2, 135.0, 131.3, 124.5, 124.3, 124.2, 123.9, 119.5, 39.8, 26.8, 26.7, 26.5, 25.8, 25.6, 17.7, 16.4, 16.1, 12.4, 12.2 E_{pc} values (mV) vs. Ag/AgNO₃ reference electrode: −1297. Regioisomeric ratio: 94:6, 3% hydroquinone impurity.

### Example 4

### Synthesis of Compound 9

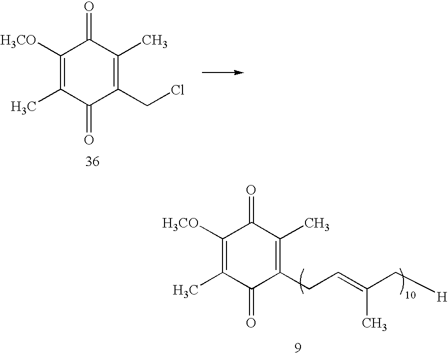

### 4.1. Synthesis of 2-(chloromethyl)-5-methoxy-3,6-dimethylcyclohexa-2,5-diene-1,4-dione 36

### 4.1.a 1,3-Dimethoxy-2,5-dimethylbenzene

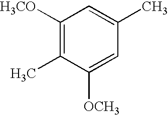

A round bottom flask equipped with a stir bar was flame-dried, argon-flushed, and charged with anhydrous THF (100 mL). Elemental potassium (6.44 g, 165.0 mmol) was then weighed out in mineral oil and added to a beaker of hexanes to wash the oil off. While under hexanes, the metal was cut into 0.5 cm×0.5 cm squares and added to the flask with THF Once all the metal was added, the flask was sealed under positive argon pressure with two septa, copper wire. Teflon tape, and parafilm. The flask was cooled to 0° C. in an ice bath. In a second dry, argon-purged flask, trimethoxytoluene (20.0 g, 11010 mmol) was dissolved in anhydrous THY (20 mL) and transferred to the first flask via cannula. The flask was then placed into a sonicator for 24 h at room temperature. When all starting material was consumed according to TLC analysis (product Rf=0.5, 10% ethyl acetate:hexanes), the reaction was removed from the sonicator, placed on a stir plate, and recooled to 0° C. MeI (9.1 mL, 148.0 mmol) was then added via syringe and stirred for 6 h. The reaction mixture underwent an immediate color change from brown-orange to white, then back to brown. The reaction was worked up by first removing any large, undissolved metal pieces from the mixture, then slowly adding isopropyl alcohol dropwise. Once all the metal was quenched, water was added to the reaction to dissolve up the salts. The organics were extracted using ethyl acetate (4×200 mL), dried over Na₂SO₄ and concentrated down to a brown oil. The product was purified by flash column chromatography on silica gel in 100% petroleum ether to afford pure, white crystals (14.0 g, 77%). Full spectral data for this compound has been reported (Azzena, U. et al., J. Org. Chem. 1990, 55, 5386-5390).

### 4.b 2,4-Dimethoxy-3,6-dimethylbenzaldehyde

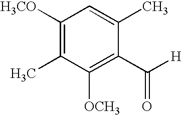

In a dry, argon-flushed, round bottom flask equipped with a stir bar, 3,5-methoxy-4-methyltoluene (16.8 g, 101.0 mmol) was dissolved in freshly distilled DMF (10.5 mL, 16.7 g, 111.0 mmol) and cooled to 0° C. Freshly distilled POCl₃ (8.52 mL, 15.0 g, 111.0 mmol) was added via syringe and the reaction was allowed to warm to rt for 1.5 h before heating to 45° C. for 18 h. Once the starting material was consumed according to TLC analysis (product Rf=0.70, 10% ether:p.ether), the reaction was cooled to 0° C. and quenched slowly with sat. NaHCO₃. The organics were extracted using ethyl acetate (3×200 mL), washed with brine, dried over Na₂SO₄ and concentrated down to an off-white solid. The product was purified by flash column chromatography on silica gel in a 5% ethyl acetate:hexanes solvent system to afford aldehyde as a white crystalline solid (18.0 g, 92%). Full spectral data for this compound has been reported (Cresp, T. et al., Australian J of Chem. 1972, 25(10) 2167).

### 4.1.c 2-Hydroxyl-4-methoxy-3,6-dimethylbenzaldehyde

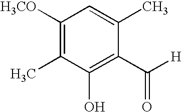

In a dry, argon-flushed, 500 mL round bottom flask equipped with a stir bar, 3,5-methoxy-1,4-methylbenzaldehyde (15.8 g, 80.9 mmol) was dissolved in anhydrous toluene (50 mL). In a second dry, argon-purged flask, AlCl₃ (12.9 g, 97.0 mmol) was suspended in anhydrous toluene (50 mL) and stirred at 0° C. The contents of the first flask were then transferred to the second flask via cannula. Immediately after the substrate was added to the AlCl₃ suspension, a thick red paste formed and was stirred at 0° C. for 8 hours. Once the starting material was consumed according to TLC analysis (product Rf=0.4, 25% ether:p. ether), the reaction was quenched slowly by adding a sat. citric acid solution (200 mL) to the reaction flask at 0° C. Several hours of stirring were required to fully break up the aluminum salts. The organics were then extracted with ethyl acetate (3×200 mL), washed with brine, dried over Na₂SO₄ and concentrated down to a yellow solid. The crude product was recrystallized from petroleum ether or purified by flash column chromatography on silica gel in a 10% ethyl acetate:hexanes solvent system to afford phenol (12.7 g, 75% yield) as yellow needle-shaped crystals. Full spectral data for this compound has been reported (Cresp, T. et al., Australian J. of Chem. 1972, 25(10) 2167).

### 4.1.d 2-(Hydroxymethyl)-5-methoxy-3,6-dimethylphenol

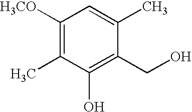

In a round bottom flask equipped with a stir bar, aldehyde (0.40 g, 2.2 mmol) was dissolved in THF (15 mL) and stirred at 0° C. To this was added NaBH₄ (0.17 g, 4.4 mmol). The heterogeneous mixture was allowed to warm to rt and stirred for 4 h, at which time no starting material remained according to TLC analysis (product Rf=0.4, 60% ethyl acetate:hexanes). Excess NaBH₄ was quenched by slowly adding HCl (6M) dropwise to the reaction until the pH was approximately 2, then solid NaCl (0.5 g, 8.55 mmol) was added to the mixture and stirred for 5-10 minutes. The organics were then extracted with chloroform (3×15 mL), dried over Na₂SO₄, and concentrated down to a clear, colorless oil (0.40 g, 95%). No further purification was necessary or possible due to the sensitive nature of the substrate.

### 4.1.e 2-(Hydroxymethyl)-5-methoxy-3,6-dimethylcyclohexa-2,5-diene-1,4-dione

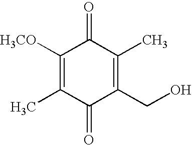

In a round bottom flask equipped with a stir bar and covered with aluminum foil to decrease exposure to light, freshly prepared potassium nitrosodisulfonate (1.39 g, 5 mmol) was dissolved in an aqueous pH 5.8 phosphate buffer (20 mL). To this rapidly stirring heterogeneous mixture was added crude diol (0.30 g, 1.67 mmol) dissolved in acetone (10 mL). The reaction was allowed to run for 34 h until the diol was completely consumed according to TLC analysis (product Rf=0.3, 25% ethyl acetate:hexanes). Brine (10 mL) and chloroform (25 mL) were added to the reaction flask and stirred for 10 minutes, after which the organics were extracted with more chloroform (3×30 mL), washed with brine (30 mL), dried over Na₂SO₄, and concentrated down to afford a thick red-orange oil containing large amounts of polymeric byproducts along with the sensitive hydroxymethylquinone. This highly impure, crude material was dried thoroughly and taken directly into the next step without further purification or exposure to light.

### 4.1.f 2-(Chloromethoxy)-5-methoxy-3,6-dimethylcyclohexa-2,5-diene-1,4-dione-36

In a flame dried, argon-flushed, round bottom flask equipped with a stir bar and covered with foil, freshly distilled PCl₃ (0.7 mL, 8 mmol) was added via syringe to freshly distilled DMF (0.62 mL, 0.8 mmol) that had been cooled to 0° C. After 0.25 h, the liquids solidified into a white crystalline salt (Vilsmeier's salt) and stirring ceased. In a second dried, argon-flushed round bottom flask, the crude hydroxymethylquinone (1.21 g, 6.7 mmol) was dissolved in anhydrous THF (10 mL). This solution was transferred via cannula to the first flask containing the solid salt, and the reaction was then allowed to warm to rt. Eventually the white salt was sufficiently dissolved and stirring resumed. After several hours the starting material was entirely consumed according to TLC (product Rf=0.20, 20% ether:hexanes), and the flask was again cooled to 0° C. Sat. NaHCO₃ was added until all of the residual Vilsmeier salt was quenched and the reaction pH was neutral. The organics were extracted with ether (3×15 mL), washed with brine, dried with Na₂SO₄, and concentrated down to a thick, red oil. The resulting chloromethylquinone was purified by flash column chromatography on silica gel in a gradient solvent system starting with 100% hexanes and ending with 10% ethyl acetate:hexanes to afford 36 as a pure orange-red oil (0.13 g, 40% over two steps). The work up and purification were done in the absence of light, the column was run quickly to avoid excess decomposition on the silica, and the final product was stored under argon at −78° C.

HRMS Calcd for C₁₀H₁₁O₃Cl: 214.0392. Found: 214.0397. Ms m/z (%): 214(74), 186(40), 179(45), 171(24), 151(73), 123(60), 91(36), 83(95), 67(73), 53(100). IR (KBr) cm⁻¹: 2966, 1655, 1444, 1375, 1282, 1256, 1147, 970, 761. ¹NMR (CDCl₃)₆: 4.456(s, 2H), 4.002 (s, 3H), 2.152 (s, 3H), 1.98 (s, 3H). ¹³C NMR (CDCl₃) δ: 185.78, 183.41, 155.94, 142.39, 138.51, 128.84, 60.99, 35.66, 11.88, 8.95.

### 4.2. Cross Coupling of the Alane 54 (n=9) and Compound 36

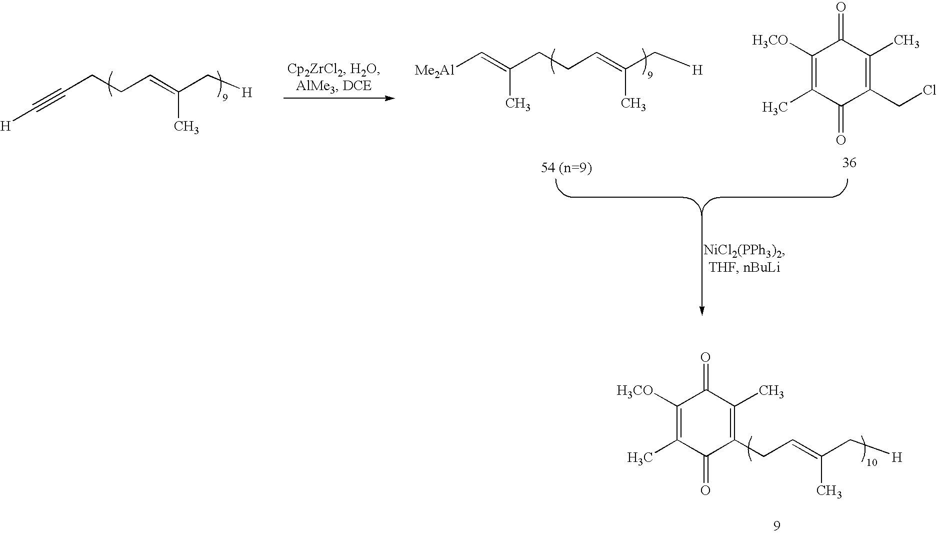

Compound 9 was prepared from the chloromethyl analog 36 and the alane 54 (n=9) following the cross coupling procedures outlined in Example 1. The following reagents were used in the specified amounts: solanesyl propyne (1.25 g, 1.94 mmol), Cp₂ZrCl₂ (0.025 g, 0.087 mmol), AlMe₃ (3.5 mL, 2.0 M in hexanes, 2.91 mmol), DCE (3 mL), H₂O (0.05 μL), NiCl₂(PPh₃)₂ (0.035 g, 0.055 mmol), nBuLi (48 μL, 2.25 M in hexanes, 0.11 mmol), 2-(chloromethoxy)-5-methoxy-3,6-dimethylcyclohexa-2,5-diene-1,4-dione (0.235 g, 1.1 mmol), THF (2 mL). Purification using flash column chromatography on silica gel in a gradient solvent system of 0-5% ethyl acetate:hexanes afforded the pure product as a bright orange-red oil (0.363 g, 39% (two steps), R_{f}=0.80, 10% ethyl acetate:hexanes).

HRMS Calcd for C₅₉H₉₀O₃: 846.6886. Found: 846.6889. Ms m/z (%): 849(21), 301(4), 219(87), 203(5), 181(29), 137(10), 121(13), 93(19), 69(19). IR (KBr) cm⁻¹: 2966, 2926, 2852, 1651, 1614, 1447, 1375, 1288, 1265, 1146, 1105, 964, 739. ¹H NMR(CDCl₃) δ: 5.137-5.051 (m, 9H), 4.937 (t, J=6.8 Hz, 1H), 3.962 (s, 3H), 3.192 (d, J=6.8 Hz, 2H), 2.104-1.944 (m, 42H), 1.751 (s, 3H), 1.687 (s, 3H), 1.630-1.584 (m, 27H). ¹³C NMR (CDCl₃) δ: 188.1, 184.2, 155.6, 143.5, 138.9, 137.5, 135.4, 135.2, 135.1, 131.4, 128.8, 124.6, 124.5, 124.4, 124.1, 119.4, 61.0, 40.0, 31.8, 27.0, 26.9, 26.7, 25.9, 25.9, 22.9, 17.9, 16.5, 16.2, 14.3, 11.9, 9.1. E_{pc} values (mV) vs. Ag/AgNO₃ reference electrode: −1223, −1467. Regioisomer ratio: 96:4

### Example 5

### Synthesis of Compound 9a

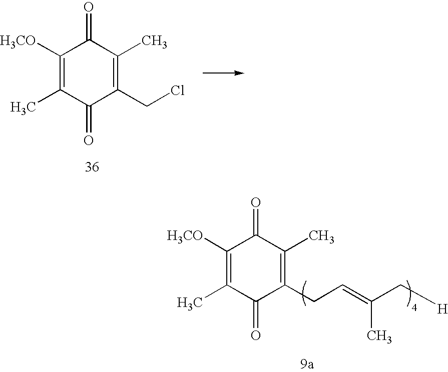

Compound 9a was synthesized using the procedures outlined in Example 4, with the difference that the alane 54 (n=9) in the cross coupling reaction is replaced with a shorter carboaluminated species 54, wherein n is 3.

HRMS Calcd for C₂₉H₄₂O₃: 438.3143. Found: 438.3134. Ms m/z (%): 438(19), 301(8), 259(5), 233(30), 219(57), 203(7), 181(23), 165(4), 69(100). ¹H NMR (CDCl₃) δ: 5.099 (m, 3H), 4.961 (t, J=7 Hz, 1H), 3.969 (s, 3H), 3.199 (d, J=6.8 Hz, 2H), 2.089-1.950 (m, 15H), 1.750 (s, 3H), 1.683 (s, 3H), 1.586-1.566 (m, 12H). ¹³C NMR (CDCl₃) δ: 188.1, 184.2, 155.6, 143.5, 138.9, 137.4, 135.3, 135.1, 131.4, 128.8, 124.6, 124.3, 124.1, 119.3, 60.9, 39.9, 31.8, 26.9, 26.8, 26.6, 25.9, 25.8, 22.9, 17.9, 16.49, 16.2, 14.3, 11.9, 9.0

### Example 6

### Synthesis of compound 11

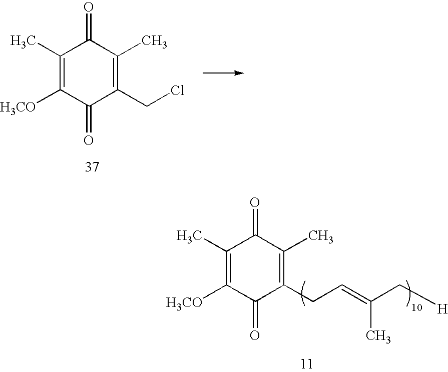

### 6.1. Synthesis of 2-(chloromethyl)-6-methoxy-3,5-dimethylcyclohexa-2,5-diene-1,4-dione (37)

### 6.1.a 2,6-Dimethylcyclohexa-2,5-diene-1,4-dione

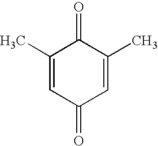

In a three-neck round bottom flask equipped with a stir bar, 1-bromo-2,3-dimethylphenol (2.19 g, 10.89 mmol) and CuCl₂-2H₂O (5.55 g, 32.69 mmol) were dissolved in DMF (50 mL, reagent grade). A reflux condenser was attached to the middle neck, and a septum was fastened to each of the outer necks, with needles inserted to deliver a constant flow of oxygen to the vented reaction flask. The reaction was heated to 60° C. for 16 h. If the reaction was not complete at this point, another equivalent of CuCl₂-H₂O was added and the mixture was stirred for an additional 16 h. When all starting material had been consumed according to TLC(Rf=0.25, 10% ethyl acetate:hexanes), the organics were extracted with ether (3×50 mL), and washed with a saturated LiCl solution to remove DMF. The combined organics were dried with Na₂SO₄ and concentrated down to a bright orange-yellow solid. The product was purified by flash column chromatography on silica gel with a 5% ethyl acetate:hexanes eluant to afford pure, bright yellow needle-shaped crystals (1.31 g, 88%). Full spectral data for this compound has been reported (Iyer, S.; Liebeskind, L. S.; J Am. Chem. Soc. 1987, 109, 2759-2770).

### 6.1.b 3,5-Dimethyl-2,6-diacetoxyphenylacetate

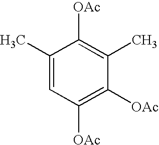

In a dry, argon-flushed round bottom flask equipped with a stir bar, 2,2-dimethylbenzoquinone (0.20 g, 1.47 mmol) was dissolved in distilled acetic anhydride (3.0 mL) and cooled to 0° C. Triflic acid (0.10 mL, 0.74 mmol) was then added via syringe slowly. The reaction exothermed and the initial yellow color changed to dark red. After allowing the reaction to warm to room temperature, TLC analysis (Rf=0.45, 60% ethyl acetate:hexanes) showed that all starting material had been consumed. A second flask containing a slurry of ice and sat. NaHCO₃ was prepared and the contents of the reaction flask were slowly poured directly into the second flask to quench out any excess acid. Once neutralized, the organics were extracted using ethyl acetate (3×10 mL), washed with brine, and dried over Na₂SO₄ affording a dark red-black solid. The crude product was purified using flash column chromatography on silica gel with a 30% ethyl acetate:hexanes solution as eluant to afford triacetate as a white crystalline solid (0.35 g, 85%). Full spectral data for this compound has been reported (Yadav, J. et al., Ten. Let., 2004, 6039-6041).

### 6.1.c 1,2,4-Trimethoxy-3,5-dimethylbenzene

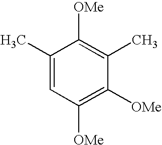

In a round bottom flask equipped with a stir bar, triacetate (10.2 g, 36.3 mmol) was dissolved in ethanol (50 mL, reagent grade). A second solution of sat. aqueous NaOH (10 mL) and Me₂SO₄ (5.0 mL, 5.4 g, 43.2 mmol) was prepared. The triacetate solution was cooled to 0° C. and the basic ethanol solution was added slowly with rapid stirring. The reaction exothermed and turned red-black. Once the exotherm resided, the reaction was allowed to warm to rt and stirred an additional 6 h until TLC analysis (Rf=0.60, 25% ethyl acetate:hexanes) showed all starting material had been consumed, at which time the mixture was recooled to 0° C. HCl (12M) was added dropwise until the pH was neutral. The organics were extracted with ethyl acetate (3×30 mL), washed with brine, dried over Na₂SO₄, and concentrated down to yield a crude black oil. The crude product was purified using flash column chromatography on silica gel in a 20% ethyl acetate:hexanes solution as eluant to afford a pure white crystalline solid (6.4 g, 90%). Full spectral data for this compound has been reported (Iyer, S.; Liebeskind, L. S.; J. Am. Chem. Soc. 1987, 109, 2759-2770).

### 6.1.d 1-(chloromethyl)-2,35-trimethoxy-4,6-dimethylbenzene

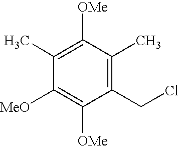

In a round bottom flask equipped with a stir bar, methoxy-protected hydroquinone (3.2 g, 16.3 mmol) was dissolved in conc. HCl (25 mL) and stirred at room temperature. Paraformaldehyde powder (1 g, 33.3 mmol) was added to the flask along with ZnCl₂ (1 g 32 mmol). The white, heterogeneous mixture was heated to reflux until all solids were in solution. The reaction was then allowed to cool to room temperature and stirred 3-4-h, until complete according to TLC analysis (Rf=0.60, 25% ether:hexanes). Distilled water was added to the reaction flask and the organics were extracted with ethyl acetate (3×30 mL), washed with brine, dried over Na₂SO₄, and concentrated down to afford a clear, yellow oil. The crude product was purified using flash column chromatography with a solution of 20% ethyl acetate:hexanes to afford the benzylic chloride (3.45 g, 86%) as a white solid.

HRMS Calcd for C₁₂H₁₇O₃: 244.0869. Found: 244.0866. Ms m/z (%): 244(100), 229(6), 201(2), 194(5), 179(21), 166(28), 151(26), 135(7), 123(22), 91(26), IR (KBr) cm⁻¹: 2939, 2831, 1711, 1637, 1466, 1404, 1271, 1196, 1117, 1067, 1002, 964, 910, 731. ¹H NMR (CDCl₃) δ: 4.710 (s, 2H), 3.910 (s, 3H), 3.814 (s, 3H), 3.676 (s, 3H), 2.315 (s, 3H), 2.216 (s, 3H). ¹³C NMR (CDCl₃) δ: 153.3, 149.9, 148.3, 128.0, 126.7, 126.6, 61.5, 60.3, 38.7, 11.6, 9.8.

### 6.1.e 2-(chloromethyl)-6-methoxy-3,5-dimethylcyclohexa-2,5-diene-1,4-dione 37

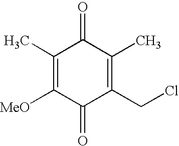

In a round bottom flask covered with foil and equipped with a stir bar, CAN (1.1 g, 1.98 mmol) was dissolved in a solution of CH₃CN: H₂O (2:1, 10 mL) and stirred at room temperature. Benzylic chloride (0.15 g, 0.50 mmol) was added to the red-orange solution and stirred at rt for 4 h. Once the oxidation was completed according to TLC(Rf=0.85, 25% ethyl acetate:hexanes), the solution was diluted with distilled water and the organics were extracted with ether (3×10 mL), washed with brine, dried over Na₂SO₄, and concentrated down to a bright yellow solid. The crude solid was purified using flash column chromatography on silica gel with a 10% ethyl acetate:hexanes solution as eluant affording pure chloromethylquinone 37 as a yellow solid (0.43 g, 82%). Care was taken to run the column quickly and limit exposure to light, and the pure product was stored at −78° C. under an inert atmosphere of argon to minimize decomposition.

HRMS Calcd for C₁₀H₁₁O₃Cl: 214.0390. Found: 214.0397. Ms m/z (%): 214(7), 178(100), 171(16), 151(42), 123(23), 107(6), 91(12), 83(13), 77(24), 67(24), 53(34). IR (KBr) cm⁻¹: 3055, 2986, 2947, 2853, 1639, 1616, 1445, 1375, 1249, 1269, 1256, 1198, 1157, 1084, 974, 893, 815, 757, 711, 699. ¹H NMR (CDCl₃)δ: 4.384 (s, 2H), 3.962 (s, 3H), 2.083 (s, 3H), 1.884 (s, 3H). ¹³C NMR (CDCl₃) δ: 187.8, 181.2, 155.3, 144.1, 137.1, 129.1, 61.1, 35.3, 12.4, 9.0.

### 6.2. Cross Coupling of the Alane 54 (n=9) and Compound 37

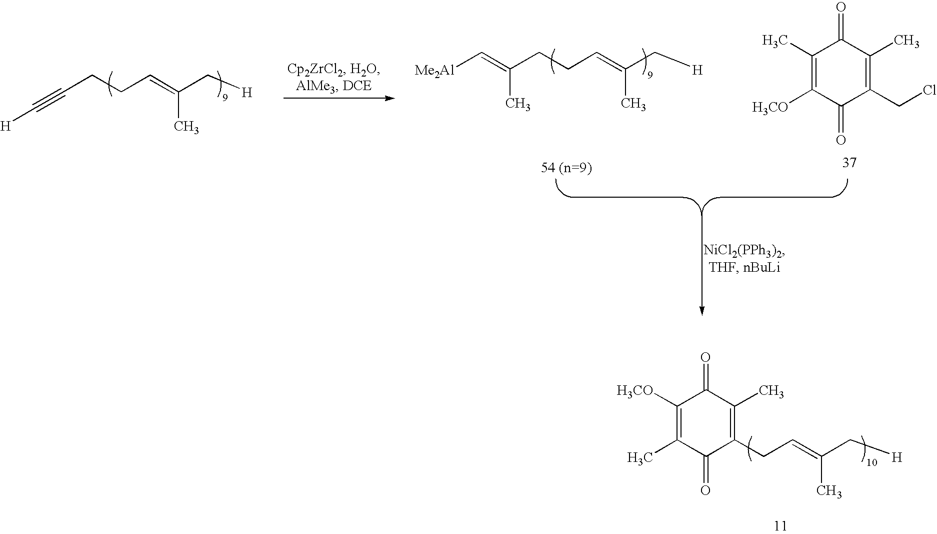

Compound 11 was synthesized from the chloromethyl analog 37 and the alane 54 (n=9) following the cross coupling procedures outlined in Example 1. The amounts of reagents were as follows: solanesyl propyne (0.600 g, 0.933 mmol), Cp₂ZrCl₂ (23 mg, 0.07 mmol), DCE (2 mL), H₂O (0.07 μL), NiCl₂(PPh₃)₂ (18 mg, 0.028 mmol), THF (2 mL), nBuLi (13 μL, 0.056 mmol), compound 37 (85 mg, 0.4 mmol). The final product was purified by flash chromatography using silica gel treated with a very small amount of Et₃N (0.25%) and a hexanes:Et₂O gradient solvent system (100% hexanes to 95%:5% hexanes: Et₂O) yielded 194 mg of a red-orange oil (47.0%). TLC: R_{f}=0.60 (5% Et₂O: hexanes).

HRMS Calcd for C₅₉H₉₀O₃: 846.6855. Found: 846.6890. Ms m/z (%): 849(2), 667(15), 257(1), 135(13), 121(16), 93(22), 81(53), 69(100), 54(22), 43(11). IR (KBr) cm⁻¹: 3053, 2986, 1649, 1612, 1421, 1265, 897, 739, 704. ¹H NMR(CDCl₃) δ: 5.121 (m, 9), 4.956 (t, J=6.4 Hz, 1H), 3.974 (s, 3H), 3.195 (d, J=6.8 Hz, 2H), 2.008-1.944 (m, 42H), 1.751 (s, 3H), 1.687 (s, 3H), 1.605 (m, 24H), 1.584 (s, 3H). ¹³C NMR(CDCl₃)δ: 188.9, 183.4, 155.6, 141.9, 140.7, 137.6, 135.4, 135.1, 131.4, 128.7, 124.6, 124.4, 124.3, 124.0, 119.3, 61.0, 39.9, 26.9, 26.7, 25.9, 25.3, 17.9, 16.5, 16.2, 12.5, 9.0, El, values (mV) vs. Ag/AgNO₃ reference electrode: −1177, −1416. Regioisomer ratio: 96:4, 2% hydroquinone impurity.

### Example 7

### Synthesis of Compound 11a

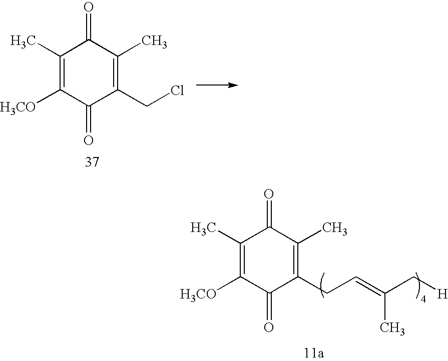

Compound 11a was synthesized using the procedure outlined in Example 6, with the difference that the alane 54 (n=9) in the cross coupling reaction is replaced with a shorter carboaluminated species 54, wherein n is 3.

HRMS Calcd for C₂₉H₄₂O₃: 438.3132. Found: 438.3134. Ms m/z (%): 438(16), 302(5), 257(6), 234(17), 219(32), 181(31), 135(10), 81(35), 69(100). ¹H NMR(CDCl₃) δ: 5.074 (m, 3H), 4.950 (t, J=7 Hz, 1H), 3.967 (s, 3H), 3.188 (d, J=7.2 Hz, 2H), 2.066-1.937 (m, 15H), 1.744 (s, 3M, 1.676 (s, 3H), 1.596 (s, 3H), 1.580 (m, 9H). ¹³C NMR (CDCl₃) δ: 188.9, 183.4, 155.6, 141.9, 140.7, 137.6, 135.4, 135.1, 131.5, 128.7, 124.6, 124.3, 124.1, 119.3, 61.0, 39.9, 26.9, 26.8, 26.7, 25.9, 25.3, 17.9, 16.5, 16.2, 12.5, 9.0.

### Example 8

### Synthesis of Compound 3

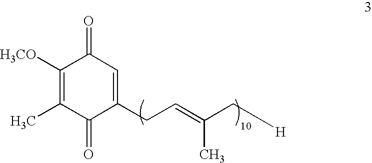

### 8.1. Synthesis of 2,4-dimethoxy-3-methylbenzaldehyde

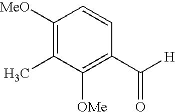

The title compound was prepared according to the procedure outlined in Example 4.1.b [synthesis of 2,4-dimethoxy-3,6-dimethylbenzaldehye]. The following reagents were used: 2,6-dimethoxytoluene (24.0 g, 158.0 mmol), POCl₃ (29.4 mL, 48.5 g, 316.0 mmol), DMF (24.5 mL, 23.1 g, 316.0 mmol). The reaction was worked up with sat. NaHCO₃, extracted with ethyl acetate (3×100 mL), rinsed with brine, dried over anhydrous Na₂SO₄, and concentrated in vacuo. The crude yellow oil was purified by flash column chromatography on silica gel in 20% ethyl acetate:hexanes to afford a white, crystalline solid (27.3 g, 96%, R_{f}=0.45 in 25% ethyl acetate:hexanes). Full spectral data for this compound has been reported (Harrowven, D. C.; Tyte, M. J. Bet. Lett. 2001, 42, 8709-8711).

### 8.2. Synthesis of 5-bromo-2,4-dimethoxy-3-methylbenzaldehyde

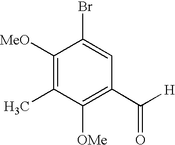

In a 250 mL round bottom flask equipped with a stir bar, 2,4-dimethoxy-3-methylbenzaldehyde (24.7 g, 137.2 mmol) was dissolved in acetic acid (150 mL). Bromine (9.13 mL, 28.5 g, 178.32 mmol) was added dropwise and the reaction was stirred at rt for 12 h. Once the reaction had gone to completion according to TLC, excess bromine was quenched using saturated, aqueous thiosulfate (20 mL). The organics were extracted in ethyl acetate (3×100 mL), rinsed with brine, dried over anhydrous Na₂SO₄, and concentrated in vacuo to afford a pale yellow solid. The crude product was then purified by flash column chromatography on silica gel using a gradient solvent system of 0-20% ethyl acetate:hexanes to afford a pure white solid (32.52 g, 92%, R_{f}=0.6, 40% ethyl acetate:hexanes).

HRMS Calcd for C₁₀H₁₁O₃Br: 257.9879. Found: 257.9878. Ms m/z (%): 258(100), 243(47), 227(22), 212(31), 162(97), 91(25), 77(55), 51(45). IR (KBr) cm⁻¹: 3003, 2941, 2864, 1686, 1579, 1454, 1383, 1265, 1096, 999, 892, 815, 738. ¹H N (CDCl₃) δ: 10.174 (s, 1H), 7.842 (s, 1H), 3.845 (s, 3H), 3.822 (s, 3H), 2.259 (s, 3H). ¹³C NMI (CDCl₃) δ: 188.22, 162.36, 161.47, 130.32, 128.13, 126.78, 113.63, 63.50, 60.56, 9.89.

### 8.3. Synthesis of S-bromo-2-hydroxy-4-methoxy-3-methylbenzaldehyde

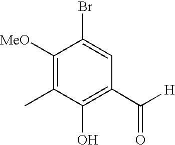

In a 250 mL round bottom flask equipped with a stir bar, 5-bromo-2,4-dimethoxy-3-methylbeazaldehyde (5.28 g, 20.49 mmol) was dissolved in CH₂Cl₂ (50 mL) and cooled to 0 ° C. To this was added BCl₃ (24.59 mL, 1 M in hexanes, 24.59 mmol). The reaction was allowed to warm to rt and stirred for 5 h. Once the reaction had gone to completion according to TLC, the contents of the flask were diluted with ether and poured over ice water. The organics were extracted in ethyl acetate (3×50 mL), rinsed with brine, dried over anhydrous Na₂SO₄, and concentrated in vacuo to an off-white solid. The crude product was then purified by flash column chromatography on silica gel using a gradient solvent system of 0-30% ethyl acetate:hexanes to afford a pure white solid (4.6 g, 92%, R_{f}=0.6, 30% ethyl acetate:hexanes).

HRMS Calcd for C₉H₉O₃Br: 243.9743. Found: 243.9735, Ms m/z (%): 244(100), 229(17), 228(19), 216(20), 214(20), 167(15), 149(60), 94(32), 65(47). IR (KBr) cm⁻¹: 3292, 3055, 2966, 2943, 2852, 1649, 1612, 1570, 1414, 1375, 1308, 1265, 1244, 1107, 1024, 993, 797, 739. ¹H NMR (CDCl₃) δ: 11.38 (s, 1H), 9.76 (s, 1H), 7.61 (s, 1H), 3.87 (s, 3H), 2.24 (s, 3H), ¹³C NMR (CDCl₃) δ: 194.9, 162.1, 161.2, 134.9, 122.3, 118.3, 107.5, 60.9, 9.2.

### 8.4. Synthesis of 4-bromo-6-(hydroxymethyl)-3-methoxy-2-methylphenol

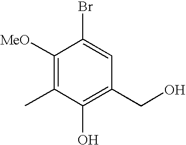

In a round bottom flask equipped with a stir bar, 5-bromo-2-hydroxy-4-methoxy-3-methylbenzaldehyde (0.24 g, 0.98 mmol) was dissolved in THY (10 mL). The light yellow solution was cooled to 0° C. and NaBH₄ (37.24 mg, 0.98 mmol) was added slowly. After 6-8 h at rt, the reaction had gone to completion and the clear solution was cooled to 0° C. Aq. HCl (1 M) was added dropwise until the solution pH=2. The organics were extracted with CHCl₃ (3×20 mL), dried over anhydrous Na₂SO₄, and concentrated in vacuo to afford a clear oil (0.24 g, 95%, R_{f} 0.2, 30% ethyl acetate:hexanes) that was pure according to TLC. The crude was taken directly on to the next step without further purification. It should be noted that CHCl₃ has been used specifically to minimize the amount of reduction byproducts carried into the next step.

### 8.5. Synthesis of 5-(hydroxymethyl)-2-methoxy-3-methylcyclohexa-2,5-diene-1,4-dione

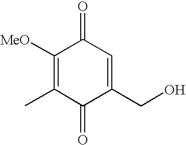

In a round bottom flask equipped with a stir bar, freshly prepared Fremy's salt (0.67 g, 2.5 mmol) was dissolved in a 1:1 solution of acetone:sodium phosphate buffer (pH=5.8) and stirred. The flask was covered with foil to avoid exposure to light. Crude 4-bromo-6-(hydroxymethyl)-3-methoxy-2-methylphenol (0.24 g, 0.98 mmol) was then dissolved in acetone and added to the stirring solution of Fremy's salt. Once the reaction was complete according to TLC, the hydroxymethyl quinone was extracted in CHCl₃ (3×50 mL) and the combined organics were dried over anhydrous Na₂SO₄, concentrated in vacuo to a dark red oil, and put on high vacuum. The crude product (R_{f}=0.5, 40% ethyl acetate:hexanes) was then dried azeotropically with benzene to remove any residual water before being carried on to the next step. It should be noted that the phosphate buffer must contain the sodium salt, as the potassium salt precipitates out during the reaction, causing rapid polymerization of the substrate. Also, failure to adequately dry the substrate at this stage results in much lower yields of the subsequent chlorination reaction due to the generation of HCl in situ.

### 8.6. Synthesis of S-(chloromethyl)-2-methoxy-3-methylcyclohexa-2,5-diene-1,4-dione (33a)

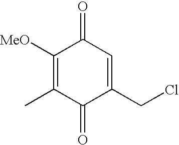

In a dry, argon-flushed round bottom flask equipped with a stir bar and covered with foil, freshly distilled DMF (0.23 mL, 0.215 g, 2.94 mmol) was stirred and cooled to 0° C. Freshly distilled PCd₃ (0.26 mL, 0.40 g, 2.94 mmol) was then added slowly to the flask of DMF. The solution was stirred 1 h, forming a white, crystalline salt (Vilsmeier's salt). Crude 5-(hydroxymethyl)-2-methoxy-3-methylcyclohexa-2,5-diene-1,4-dione was then dissolved in freshly distilled THF and added to the flask containing the salt. After 4-6 h of stirring at rt, the reaction was complete. Ether (15 mL) was added and the mixture was poured over a filter of silica gel to separate the organics from excess reagent and polymeric material. The resulting bright yellow filtrate was concentrated in vacuo to a dark, orange-red oil. Care was taken to avoid exposure to light, water, or heat, as this would result in polymerization. The product was immediately purified by flash column chromatography on silica gel in a gradient solvent system of 0-15% ether:hexanes to afford a pure yellow solid (0.076 g, 38.7%, R_{f}=0.85, 30% ethyl acetate:hexanes). It should be noted that if, during the generation of the Vilsmeier salt, no solid formed or the solid had a yellow tint, the flask was discarded. The yellow color and failure to crystallize were generally indications that excess HCl resulting from a wet reagent was present. Under such conditions, the amount of polymeric byproducts was substantially increased.

HRMS Calcd for C₉H₉O₃Cl: 200.0232. Found, 200.0240. Ms m/z (%): 200(44), 165(37), 164(42), 137(31), 134(100), 109(43), 83(59), 67(94), 55(37). IR (KBr) cm⁻¹: 2955, 2926, 2855, 1653, 1608, 1447, 1377, 1321, 1290, 1209, 1146, 739. ¹H NMR (CDCl₃) δ: 6.725 (s, 1H), 4.401 (s, 2H), 4.038 (s, 3H), 1.948 (s, 3H). ¹³C NMR (CDCl₃) 6; 186.66, 183.28, 155.94, 143.63, 132.11, 128.71, 61.15, 39.58, 8.92.

### 8.7., Synthesis of Compound 3

The title compound was prepared from the chloromethylene analog 33a and 54 (n=9) according to the procedures outlined in Example 1. The following reagents were used in the specified amounts: 5-(chloromethyl)-2-methoxy-3-methylcyclohexa-2,5-diene-1,4-dione 33a (84.0 mg, 0.42 mmol), solanesyl propyne (0.5 g, 0.765 mmol), Cp₂ZrCl₂ (0.011 g, 0.038 mmol), AlMe₃ (0.57 mL, 2 M solution in toluene, 1.15 mmol), MAO (0.02 mL, 10% w/w solution in toluene, 0.038 mmol), NiCl₂(PPh₃)₂ (24.84 mg, 0.038 mmol), THF (2 mL), nBuLi (0.03 mL, 2.5 M solution in hexanes, 0.076 mmol), CAN (0.23 g, 0.420 mmol), CH₃CN: H₂O (10:1, 20 mL). The resulting oxidized final product was purified by flash column chromatography on silica gel in a gradient solvent system of 0-10% ether:petrol ether to afford a pure orange-red oil (99.0 mg, 28% (3 steps), R_{f}=0.80, 10% ether: hexanes).

HRMS Calcd for C₅₈H₈₈O₃Na: 855.6638. Found: 855.6625. Ms m/z (counts); 858(12), 857(35), 856(57), 836(25), 835(80), 437(83), 413(98), 393(151), 381(114) IR (KBr) cm⁻¹: 2963, 2920, 2851, 1649, 1607, 1448, 1381, 1321, 1209, 1144. ¹H NMR (CDCl₃) 6; 6.347 (s, 1H), 5.139-5.107 (m, 10H), 4.012 (s, 3H), 3.122 (d, J=7.2 Hz, 2.105-1.951 (m, 39H), 1.688 (s, 6H), 1.686-1.607 (m, 27H) ¹³C NMR (CDCl₃) 6; 188.53, 184.04, 155.63, 148.52, 140.04, 135.61, 135.09, 131.42, 130.67, 128.98, 124.57, 124.42, 123.95, 118.13, 61.02, 39.93, 27.60, 26.86, 26.62, 25.91, 17.88, 16.33, 16.21, 9.07. E_{pc} values (mV) vs. Ag/AgNO₃ reference electrode: −1091, −1406. Regioisomer ratio: 90:10, 7% hydroquinone impurity.

### Example 9

### Synthesis of Compound 15

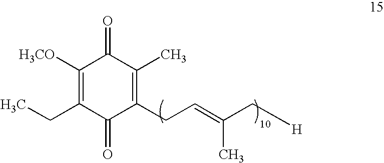

### 9.1. Synthesis of 2-ethyl-1,3-dimethoxy-5-methylbenzene

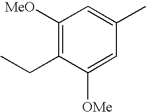

The title compound was prepared according to the procedure outlined in Example 4.1.a (compound 9). The following reagents were used in the specified amounts: trimethoxytoluene (5.0 g, 27.5 mmol), potassium (3.2 g, 82.4 mmol), ethyl iodide (2.7 mL, 3.9 g, 35.8 mmol), THF (50 mL). The crude red-brown oil was purified by flash column chromatography on silica gel in a gradient solvent system of 0-15% ethyl acetate:hexanes to afford a pure, clear oil (3.4 g, 69%, R_{f}=0.9, 14% ethyl acetate:hexanes). Full spectral data for this compound has been reported previously (Azzena, U.; Denurra, T.; Melloni, G.; Piroddi, A. M.; J Org. Chem. 1990, 55, 5386-5390).

### 9.2. Synthesis of 3-ethyl-2,4-dimethoxy-6-methylbenzaldehyde

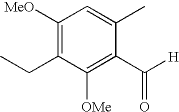

The title compound was prepared according to the procedure outlined in Example 8.1. (synthesis of 2,4-dimethoxy-3-methylbenzaldehyde). The following reagents were used in the specified amounts, 2-ethyl-1,3-dimethoxy-5-methylbenzene (5.0 g, 27.8 mmol), POCl₃ (5.2 mL, 8.5 g, 55.6 mmol), DMF (8.6 mL, 8.1 g, 111.0 mmol). The resulting brown oil was purified by flash column chromatography on silica gel in a gradient solvent system of 5%-25% ethyl acetate:hexanes to afford a pure, yellow oil (4.51 g, 78%, R_{f}=0.65, 14% ethyl acetate:hexanes).

HRMS Calcd for C₁₂H₁₆O₃Na: 231.1002. Found: 231.0991 Ms m/z (counts): 231(207), 210(20), 209(133), 17918), 130(12), 102(10). IR (KBr) cm⁻¹: 2964, 2873, 1682, 1595, 1555, 1454, 1379, 1323, 1296, 1218, 1128, 1089, 1058, 1014, 918, 792, 575, 521, ¹H NMR (CDCl₃) δ: 10.394 (s, 1H), 6.506 (s, 1H), 3.885 (s, 3H), 3.842 (s, 3H), 2.645 (q, J=7.2 Hz, 2H), 2.598 (s, 3H), 1.143 (t, J=7.2 Hz, 3H). ¹³C NMR (CDCl₃) δ: 191.55, 164.08, 162.65, 141.84, 123.98, 121.13, 110.12, 64.55, 55.84, 22.18, 16.72, 14.36.

### 9.3. Synthesis of 3-ethyl-2-hydroxy-4-methoxy-6-methylbenzaldehyde

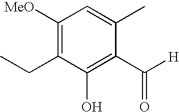

The title compound was prepared according to the procedure outlined in Example 8.3 (synthesis of 5-bromo-2-hydroxy-4-methoxy-3-methylbenzaldehyde). The following reagents were used in the specified amounts: 3-ethyl-2,4-dimethoxy-6-methylbenzaldehyde (4.61 g, 22.16 mmol), BCl₃ (44.32 mL, 1 M in hexanes, 6.78 g, 44.32 mmol), CH₂Cl₂ (100 mL). The resulting white solid was purified by flash column chromatography on silica gel in a gradient solvent system of 5%-20% ethyl acetate:hexanes to afford yellow, needle-shaped crystals (3.93 g, 91%, R_{f}=0.75 in 20% ethyl acetate:hexanes).

HRMS Calcd for C₁₁H₁₄O₃: 194.0946. Found: 194.0943. Ms m/z (%): 194(62), 179(100), 164(12), 149(15), 77(11). IR (KBr) cm⁻¹: 3488, 2970, 2876, 1624, 1504, 1464, 1412, 1358, 1294, 1225, 1144, 1084, 1020, 914, 746, 649. ¹H NMR (CDCl₃) δ: 12.385 (s, 1H), 10.122 (s, 1H) 6.279 (s, 1H), 3.889 (s, 3H), 2.620 (q, J=7.2 Hz, 2H), 2.560 (s, 3H), 1.081 (t, J=7.2 Hz, 3H). ¹³C NMR (CDCl₃) δ: 193.45, 164.12, 162.81, 142.00, 117.67, 113.73, 105.46, 55.89, 18.69, 15.51, 13.43.

### 9.4, Synthesis of 2-ethyl-6-(hydroxylmethyl)-3-methoxy-5-methylphenol

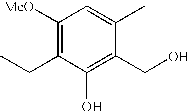

The title compound was prepared according to the procedure outlined in Example 8.4. (synthesis of 4-bromo-6-(hydroxymethyl)-3-methoxy-2-methylphenol). The following reagents were used in the specified amounts: 3-ethyl-2-hydroxy-4-methoxy-6-methylbenzaldehyde (3.93 g, 20.26 mmol), NaBH₄ (0.77 g, 20.26 mmol), THF (100 mL). The resulting clear oil (3.92 g, 95%, R_{f}=0.55 in 40% ethyl acetate:hexanes) was taken on to the next step crude.

### 9.5. Synthesis of 2-ethyl-t6-(hydroxymethyl)-3-methoxy-5-methylcyclohexa-2,5-diene-1,4-dione

The title compound was prepared according to the procedure outlined in Example 4.1.e [synthesis of 2-(hydroxy ethyl)-5-methoxy-3,6-dimethylcyclohexa-2,5-diene-1,4-dione]. The following reagents were used in the specified amounts: 2-ethyl-6-(hydroxylmethyl)-3-methoxy-5-methylphenol (3.9 g, 20.0 mmol), Fremy's salt (21.7 g, 81.04 mmol), acetone (200 mL), pH 5.8 phosphate buffer (200 mL). The resulting bright red-orange solid (4.2 g impure material, R_{f}=0.45, 40% ethyl acetate:hexanes) was azeotropically dried with benzene and taken on to the next step crude.

### 9.6. Synthesis of 2-(chloromethyl)-6-ethyl-5-methoxy-3-methylcyclohexa-2,5-diene-1,4-dione (36a)

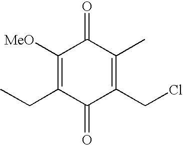

The title compound was prepared according to the procedure outlined in Example 4.1.f [synthesis of 2-(chloromethoxy)-5-methoxy-3,6-dimethylcyclohexa-2,5-diene-1,4-dione]. The following reagents were used in the specified amounts: 2-ethyl-6-(hydroxymethyl)-3-methoxy-5-methylcyclohexa-2,5-diene-1,4-dione (4.2 g impure crude material), PCl₃ (5.3 mL, 8.4 g, 60.8 mmol), DMF (4.7 mL, 4.4 g, 60.8 mmol), THF (40 mL). The resulting red-orange crude oil was purified by flash column chromatography on silica gel in a gradient solvent system of 0%-10% ethyl acetate:hexanes to afford a bright orange oil (1.93 g, 42% (three steps), R_{f}=0.65, 10% ethyl acetate:hexanes).

HRMS Calcd for C₁₁H₁₃O₃Cl; 228.0542. Found: 228.0553. Ms m/z (%): 228(42), 213(18), 193(84), 192(63), 174(68), 119(20), 91(37), 81(40), 53(100). IR (KBr) cm⁻¹: 2939, 2876, 1653, 1612, 1458, 1447, 1381, 1353, 1323, 1252, 1146, 1055, 995, 866, 762. ¹H NMR (CDCl₃) δ: 4.450 (s, 2H), 4.000 (s, 3H), 2.481 (q, J=7.2 Hz, 2H), 2.143 (s, 3H), 1.049 (t, J=7.2 Hz, 3H). ¹³C NMR (CDCl₃) δ: 185.47, 183.89, 155.73, 142.48, 138.65, 134.48, 61.30, 35.73, 17.02, 13.62, 12.02.

### 9.7. Synthesis of Compound 15

The title compound was prepared from 36a and 54 (n=9) according to the procedures outlined in Example 1. The following reagents were used in the specified amounts: 2-(chloromethyl)-6-ethyl-5-methoxy-3-methylcyclohexa-2,5-diene-1,4-dione (0.174 g, 0.76 mmol), solanesyl propyne (0.5 g, 0.765 mmol). Cp₂ZrCl₂ (0.011 g, 0.038 mmol), AlMe₃ (0.58 mL, 2 M solution in toluene, 0.083 g, 1.15 mmol), MAO (0.02 mL, 0.0029 g, 0.038 mmol), NiCl₂(PPh₃)₂ (0.025 g, 0.038 mmol), THF (2 mL), nBuLi (0.03 mL, 2.5 M solution in hexanes, 0.076 mmol), CAN (1.0 g, 1.9 mmol), CH₃CN: H₂O (10:1, 40 mL total). The oxidized final product was purified by flash column chromatography on silica gel in a gradient solvent system of 0-10% ethyl acetate, hexanes to afford a pure orange-red oil (0.209 g, 32% (three steps), R_{f}=0.7, 10% ethyl acetate, hexanes).

HRMS Calcd for C₆₀H₉₂O₃: 860.7065. Found: 860.7046. Ms m/z(%): 863(18), 861(13), 233(91), 195(40), 135(19), 121(23), 95(29), 69(100), 54(32). IR (KBr) cm⁻¹: 2964, 2922, 2851, 1647, 1610, 1448, 1381, 1350, 1323, 1254, 1146, 1045, 989, 838. ¹H NMR (CDCl₃) δ: 5.138-5.052 (m, 9H), 4.953 (t, J=1.2 Hz, 1H), 3.976 (s, 3H), 3.199 (d, J=7.2 Hz, 2H), 2.460 (q, J=7.2 Hz, 2H), 2.105-1.936 (m, 39H), 1.752 (s, 3H), 1.688 (s, 6H), 1.605-1.582 (m, 24H), 1.404-1.340 (m, 4H), 1.044 (t, J=7.2 Hz, 3H). ¹³C NMR (CDCl₃) δ: 187.62, 184.57, 155.30, 143.51, 138.96, 137.46, 135.40, 135.14, 134.30, 131.45, 124.60, 124.45, 125.36, 124.07, 119.42, 61.20, 39.95, 26.90, 26.72, 25.92, 25.83, 17.90, 17.03, 16.53, 16.23, 13.74, 11.92. E_{pc} values (mV) vs. Ag/AgNO₃ reference electrode: −1126, −1374. Regioisomeric ratio: 96:4, 2% hydroquinone impurity.

### Example 10

### Synthesis of Compound 17

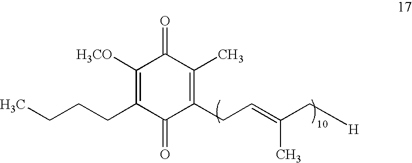

### 10.1. Synthesis of 2-butyl-1,3-dimethoxy-5-methylbenzene

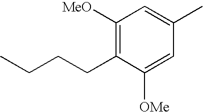

The title compound was prepared according to the procedure outlined in Example 4.1.a [synthesis of 1,3-dimethoxy-2,5-dimethylbenzene]. The following reagents were used in the specified amounts: trimethoxytoluene (5.0 g, 27.5 mmol), potassium (3.22 g, 82.42 mmol), n-butyl bromide (3.86 mL, 4.9 g, 35.75 mmol), THF (50 mL). The resulting red-brown oil was vacuum distilled to afford a pure, clear oil (4.4 g, 77%, R_{f}=0.9, 14% ethyl acetate:hexanes).

HRMS Calcd for C₁₃H₂₀O₂: 208.1455. Found: 208.1450. Ms m/z(%): 208(18), 165(100), 105(24), 91(6), 79(5), 43(8), IR (KBr) cm⁻¹: 2995, 2955, 2858, 2835, 1608, 1587, 1456, 1416, 1313, 1247, 1205, 1140, 1076, 970, 812. ¹H NMR(CDCl₃)δ: 6.401 (s, 2H), 3.826 (s, 6H), 2.633 (t, J=7.2 Hz, 2H), 2.370 (s, 3H), 1.497-1.353 (m, 4H), 0.952 (t, J=7.2 Hz, 3H). ¹³C NMR (CDCl₃) δ: 158.28, 136.53, 116.72, 104.77, 55.83, 31.94, 23.10, 22.68, 22.18, 14.30.

### 10.2, Synthesis of 3-butyl-2,4-dimethoxy-6-methylbenzaldehyde

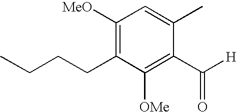

The title compound was prepared according to the procedure outlined in Example 4.1.b [synthesis of 2,4-dimethoxy-3,6-dimethylbenzaldehye]. The following reagents were used: 2-butyl-1,3-dimethoxy-5-methylbenzene (0.5 g, 2.4 mmol), POCl₃ (0.45 mL, 0.74 g, 4.8 mmol), DMF (0.37 mL, 0.35 g, 4.8 mmol). The resulting brown oil was purified by flash column chromatography on silica gel in a gradient solvent system of 0-25% ethyl acetate:hexanes to afford a pure, yellow oil (0.42 g, 68%, R_{f}=0.5, 14% ethyl acetate:hexanes ).

HRMS Calcd for C₁₄H₂₀O₃Na: 259.1317. Found: 259.1304. Ms m/z (counts): 259(133), 237(36), 181(10), 133(8). IR(KBr) cm⁻¹: 2957, 2860, 1682, 1596, 1462, 1379, 1323, 1299, 1220, 1207, 1130, 1072, 1016, 838, 789, 577. ¹H NMR (CDCl₃) δ: 10.386 (s, 1H), 6.496 (s, 1H), 3.871 (s, 3H), 3.827 (s, 3H), 2.614-2.576 (m, 5H), 1.529-1.453 (m, 2H), 1.427-1.336 (m, 2H), 0.934 (t, J=7.2 Hz, 3H). ¹³C NMR (CDCl₃) δ: 191.53, 164.26, 162.70, 141.77, 122.74, 121.07, 110.07, 64.42, 55.81, 32.01, 23.17, 23.12, 22.15, 14.14.

### 10.3. Synthesis of 3-butyl-2-hydroxy-4-methoxy-6-methylbenzaldehyde

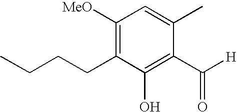

The title compound was prepared according to the procedure outlined in Example 8.3. [synthesis of 5-bromo-2-hydroxy-4-methoxy-3-methylbenzaldehyde]. The following reagents were used in the specified amounts: 3-butyl-2,4-dimethoxy-6-methylbenzaldehyde (3.14 g, 13.3 mmol), BCl₃ (19.95 mL, 1 M in hexanes, 3.05 g, 19.95 mmol), CH₂Cl₂ (40 mL). The resulting yellow solid was purified by flash column chromatography on silica gel in a gradient solvent system of 0%-15% ethyl acetate:hexanes to afford yellow, needle-shaped crystals (3.00 g, 95%, R_{f}=0.8, 20% ethyl acetate:hexanes).

HRMS Calcd for C₁₃H₁₈O₃ 222.1262. Found: 222.1256. Ms m/z (%): 222(25), 193(10), 179(100), 149(14), 91(7). IR (KBr) cm⁻¹: 3417, 2957, 2860, 1628, 1573, 1498, 1463, 1412, 1360, 1294, 1256, 1225, 1144, 1020, 822, 733. ¹H NMR (CDCl₃) δ; 12.389 (s, 1H), 10.120 (s, 1H), 6.276 (s, 1H), 3.882 (s, 3H), 2.594 (t, J=7.2 Hz, 2H), 2.560 (s, 3H), 1.499-1.307 (m, 4H), 0.921 (t, J=7.2 Hz, 3H). ¹³C NMR (CDCl₃) δ: 193.43, 164.30, 163.03, 141.97, 116.43, 113.69, 105.43, 55.87, 31.16, 22.98, 21.86, 18.70, 14.24.

### 10.4. Synthesis of 2-butyl-6-(hydroxymethyl)-3-methoxy-5-methylphenol

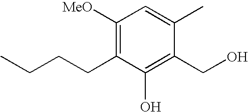

The title compound was prepared according to the procedure outlined in Example 8.4. [synthesis of 4-bromo-6-(hydroxymethyl)-3-methoxy-2-methylphenol]. The following reagents were used in the specified amounts-3-butyl-2-hydroxy-4-methoxy-6-methylbenzaldehyde (0.85 g, 3.83 mmol), NaBH₄ (0.142 g, 3.83 mmol), THF (20 mL). The resulting clear oil (0.85 g, 98%, R_{f} 0.55, 43% ethyl acetate:hexanes) was taken on to the next step without further purification.

### 10.5. Synthesis of 2-butyl-6(hydroxymethyl)-3-methoxy-5-methylcyclohexa-2,5-diene-1,4-dione

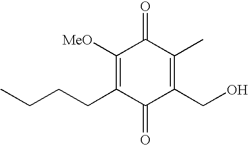

The title compound was prepared according to the procedure outlined in Example 4.1.e [synthesis of 2-(hydroxymethyl)-5-methoxy-3,6-dimethylcyclohexa-2,5-diene-1,4-dione]. The following reagents were used in the specified amounts: 2-butyl-6-(hydroxymethyl)-3-methoxy-5-methylphenol (0.85 g, 3.8 mmol), Fremy's salt (4.07 g, 15.18 mmol), acetone (20 mL), pH 5.8 phosphate buffer (20 mL). The resulting dark red solid (1.02 g (impure), R_{f}=0.45, 43% ethyl acetate, hexanes) was azeotropically dried with benzene and taken on to the next step crude.

### 10.6. Synthesis of 2-butyl-6-(chloromethyl)-3-methoxy-5-methylcyclohexa-2,5-diene-1,4-dione (36b)

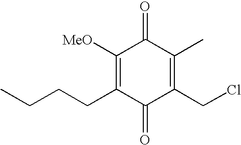

The title compound was prepared according to the procedure outlined in Example 4.1.f [synthesis of 2-(chloromethoxy)-5-methoxy-3,6-dimethylcyclohexa-2,5-diene-1,4-dione 36]. The following reagents were used in the specified amounts: 2-butyl-6-(hydroxymethyl)-3-methoxy-5-methylcyclohexa-2,5-diene-1,4-dione (1.02 g (impure)), PCl₃ (0.99 ml, 1.56 g, 11.4 mmol), DMF (0.89 mL, 0.83 g, 11.4 mmol), THF (25 mL). The resulting dark red oil was purified by flash column chromatography on silica gel in a gradient solvent system of 0-10% ethyl acetate:hexanes to afford a red-orange oil (0.33 g, 34% (three steps), R_{f}=0.8, 29% ethyl acetate:hexanes).

HRMS Calcd for C₁₃H₁₇O₃Cl: 256.0878. Found: 256.0866. Ms m/z(%): 256(21), 222(16), 214(31), 205(48), 191(35), 185(22), 178(57), 150(31), 107(20), 91(47), 77(44), 53(100). IR (KBr) cm⁻¹: 2957, 2932, 1653, 1610, 1448, 1279, 1256, 1219, 1146, 1070, 762. ¹H NMR (CDCl₃) δ: 4.451 (s, 2H), 3.997 (s, 3H), 2.464 (t, J=7.2 Hz, 2H), 2.148 (s, 3H), 1.422-1.339 (m, 4H), 0.919 (t J=7.2 Hz, 3H). ¹³C NMR (CDCl₃) δ: 185.63, 183.82, 155.94, 142.46, 138.61, 133.32, 61.23, 35.73, 31.23, 23.33, 23.06, 14.05, 12.00.

### 10.6. Synthesis of Compound 17

The title compound was prepared from 36b and 54 (n=9) according to the procedures outlined in Example 1. The following reagents were used in the specified amounts: 2-(chloromethyl)-6-butyl-5-methoxy-3-methylcyclohexa-2,5-diene-1,4-dione (0.100 g, 0.39 mmol), solanesyl propyne (0.5 g, 0.765 mmol), Cp₂ZrCl₂ (0.011 g, 0.038 mmol), AlMe₃ (0.58 mL, 2 M solution in toluene, 0.083 g, 1.15 mmol), MAO (0.02 mL, 0.0029 g, 0.038 mmol), NiCl₂(PPh₃)₂ (24.8 mg, 0.038 mmol), THF (2 mL), nBuLi (0.03 mL, 2.5 M solution in hexanes, 0.076 mmol), CAN (0.21 g, 0.39 mmol), CH₃CN: H₂O (10:1, 20 mL total). The resulting oxidized final product was purified by flash column chromatography on silica gel in a gradient solvent system of 0-10% ethyl acetate:hexanes to afford a pure orange-red oil (0.17 g, 49% (three steps), R_{f}=0.8, 10% ethyl acetate:hexanes).

HRMS Calcd for C₆₂H₉₆O₃: 888.7399. Found, 888.7359. Ms m/z (%): 891(100), 669(35), 531(12), 327(28), 150(11), 122(23), 81(75), 69(100), 54(25). IR (KBr) cm⁻¹: 2962, 2922, 2853, 1651, 1610, 1447, 1381, 1263, 1148, 1103, 1006, 839, 741. ¹H NMR (CDCl₃) δ: 5.139-5.070 (m, 9H), 4.951 (t, J=1.2 Hz, 1H), 3.980 (s, 3H), 3.196 (d, J=7.2 Hz, 2H), 2.437 (t, J=7.2 Hz, 2H), 2.106-1.938 (m, 42H), 1.736 (s, 3H), 1.689 (s, 3H), 1.607-1.584 (m, 24H), 1.371-1.340 (m, 4H), 0.917 (t, J=7.2 Hz, 3H). ¹³C NMR(CDCl₃) δ: 187.84, 184.54, 155.54, 143.50, 138.97, 137.47, 135.42, 135.16, 133.14, 131.48, 124.60, 124.45, 124.08, 119.43, 61.16, 39.96, 32.21, 31.39, 29.93, 26.91, 26.74, 25.93, 23.66, 23.40, 23.13, 1791, 16.56, 16.24, 14.13, 11.95. E_{pc} values (mV) vs. Ag/AgNO₃ reference electrode: −1138, −1326. Regioisomeric ratio: 94:6, 3% hydroquinone impurity.

### Example 11

### Synthesis of Compound 23 (Z¹═H)

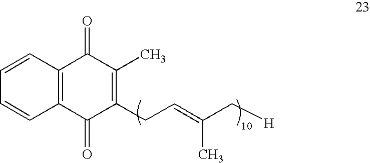

### 11.1. Synthesis of 2-(chloromethyl)-3-methylnaphthalene-1,4-dione (35a)

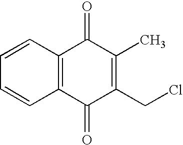

The title compound was prepared according to the procedures outlined in Example 3.1.b.) (preparation of 1-(chloromethyl)-2,4-,dimethoxy-3,4,6-trimethylbenzene) The following reagents were used in the specified amounts: naphthoquinone (5.0 g, 29.0 mmol), ZnCl₂ (3.94 g, 29.0 mmol), concentrated HCl (10 mL), paraformaldehyde (1.98 g, 58.0 mmol). Flash column chromatography on silica gel in 5% ether:hexanes afforded the product as a yellow solid (4.9 g, 77%, R_{f}=0.8, 10% ethyl acetate:hexanes). Full spectral data for this compound has been reported (Lipshutz, B. H.; Kim, S-k.; Mollard, P.; Stevens, K. L. Tetrahedron, 1998, 54, 1241-1253).

### 11.1. Synthesis of Compound 23

The title compound was prepared from 35a and 54 (n=9) according to the procedures outlined in Example 1. The following reagents were used in the specified amounts: 2-(chloromethyl) naphthalene (0.18 g, 0.8 mmol), solanesyl propyne (0.654 g, 1.0 mmol), Cp₂ZrCl₂ (0.011 g, 0.038 mmol), AlMe₃ (0.75 mL, 2 M solution in toluene, 1.5 mmol), MAO (0.064 mL, 0.073 g, 0.10 mmol), NiCl₂(PPh₃)₂ (26.2 mg, 0.04 mmol), THF (2 mL), nBuLi (30 μL, 2.5 M solution in hexanes, 0.08 mmol, 30 μL). The final product was purified by flash column chromatography on silica gel in a gradient solvent system of 0-10% ether:pet. ether to afford a pure yellow solid (0.53 g, 78% (two steps), R_{f}=0.80, 2% ether:hexanes).

HRMS Calcd for C₆₁H₈₈O₂: 852.6820. Found: 852.6843. Ms m/z (%); 852(3), 224(11), 203(6), 187(12), 161(10), 137(15), 135(26), 120(15), 109(23), 69(100), 55(32). IR (KBr) cm⁻¹: 2964, 2920, 2853, 1661, 1447, 1381, 1296, 910, 735. ¹H NMR (CDCl₃) δ: 8.100-8.072 (m, 2H), 7.699-7.676 (m, 2H), 5.127-5.017 (m, 10H), 3.382 (d, J=7.2 Hz, 2H), 2.200 (s, 3H), 2.075-1.933 (m, 36H), 1.809 (s, 3H), 1.690 (s, 3H), 1.610-1.539 (m, 27H). ¹³C NMR(CDCl₃) 6; 185.60, 184.66, 146.30, 143.50, 137.72, 135.39, 135.09, 133.50, 133.44, 132.31, 131.41, 126.49, 126.37, 124.59, 124.44, 124.32, 124.02, 119.26, 39.92, 26.88, 26.67, 26.19, 25.91, 17.88, 16.62, 16.23, 12.87. E_{pc} values (mV) vs. Ag/AgNO₃ reference electrode: −1435. Regioisomer ratio: 92:8, 1% hydroquinone impurity.

### Example 12

### Preparation of the CoQ-dopamine Adduct 39a

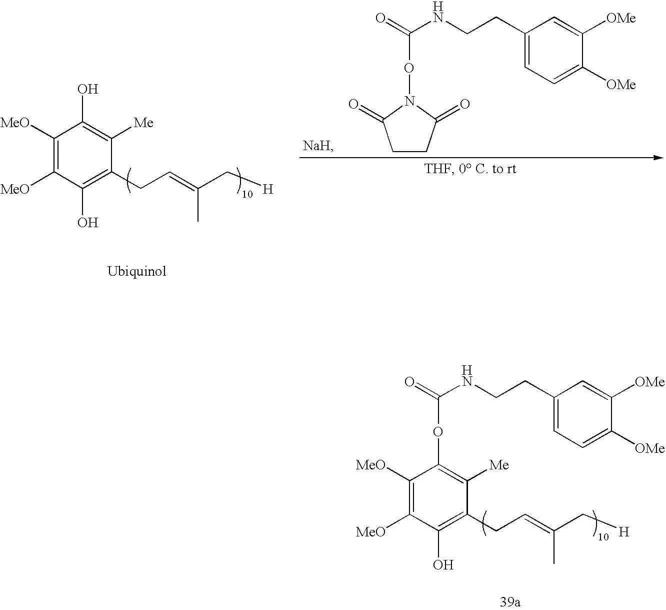

To a flame dried argon purged rb flask was added ubiquinol (3.0 g, 3.5 mmol) and THE (35 mL) and the reaction was cooled to 0° C. 95% NaH (100 mg, 3.9 mmol) was added in one portion and the reaction was stirred until gas evolution ceased (15 minutes). The dopamine carbamate (1.2 g, 3.5 mmol) was added neat and the reaction was allowed to warm from 0° C. to rt. After 3 h the reaction was diluted with Et₂O (50 mL) and washed with aqueous NaHCO₃ (2×50 mL). The organic layer was dried over anhydrous MgSO₄ and concentrated in vacuo. The residue was purified by silica gel chromatography (50% EtOAc/hex), isolating 2.4 g (64%) of the CoQ-dopamine adduct 39a. R_{f} 0.63 (50% EtOAc/hex).

¹H NMR (400 MHz, CDCl₃): δ=6.84 (d, J=8 Hz, 1H), 6.78 (d, J=8 Hz, 1H), 6.77 (s, 1H), 5.75 (s, 1H), 5.72 (s, 1H), 5.13-5.06 (b m, 10H), 3.93 (s, 3H), 3.91 (s, 3H), 3.88 (s, 3H), 3.84 (s, 3H), 3.53 (t, J=6 Hz, 1H), 3.34 (d, J=6 Liz, 1H), 3.22 (d, J=6 Hz, 1H), 2.84 (dd, J=7 Hz, J=10 Hz, 2H), 2.14 (s, 3H), 2.10-2.01 (b m, 36H), 1.73 (s, 3H), 1.69 (s, 3H), 1.61 (b s, 24H), 1.58 (s, 3H). HRMS (ESI+/TOF): m/z calcd for C₇₀H₁₀₅O₇N, (M+H)⁺1072.7934, found 1072.7935.

### Example 13

### Preparation of the CoQ-dopamine Adduct 43a

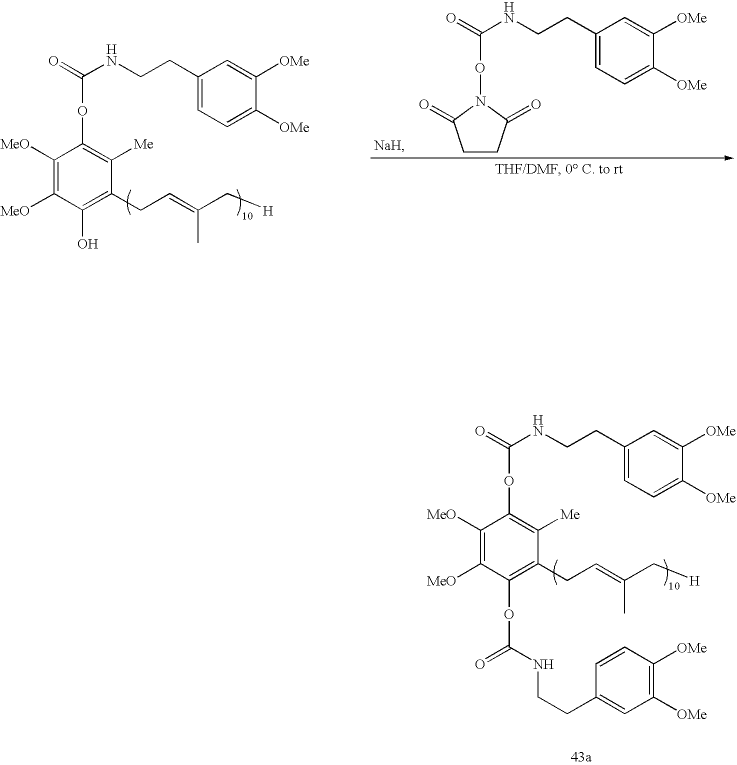

To a flame dried argon purged rb flask was added CoQ-dopamine adduct 39a (1.0 g, 1.0 mmol), THF (5 mL) and DMF (5 mL). The reaction was cooled to 0° C. 95% NaH (25 mg, 1.1 mmol) was added in one portion followed by the dopamine carbamate (330 mg, 1.0 mmol). The reaction was allowed to warm from 0° C. to rt. After 3 h the reaction was concentrated in vacuo and the crude mixture was diluted with aqueous NaHCO₃ and extracted with Et₂O (3×20 mL). The combined organic extracts were washed with brine and dried over anhydrous MgSO₄. The solvent was removed in vacuo and the residue was purified by silica gel chromatography (50% EtOAc/hex), isolating 970 mg (76%) of the 2:1 CoQ-dopamine adduct 43a. R_{f} 0.35 (50% EtOAc/hex).

¹H NMR (400 MHz, CDCl₃): δ=6.84 (d, J=8 Hz, 2H), 6.78 (d, J=8 Hz, 2H), 6.77 (s, 1H), 5.13-5.06 (b m, 10H), 4.22 (b s, 2H), 3.90-3.88 (b s, 12H), 3.86-3.84 (b s, 6H), 3.52 (t, J=6 Hz, 4H), 3.22 (d, J=6 Hz, 2H), 2.84 (dd, J=7 Hz, J=10 Hz, 4H), 2.11-2.02 (b m, 36H), 2.05 (s, 3H), 1.71 (s, 3H), 1.69 (s, 3H), 1.61 (b s, 24H), 1.58 (s, 3H). HRMS (ESI+/TOF): m/z calcd for C₁₈H₁₂₈O₁₀N₂, (M+H)⁺1279.8829, found 1279.8221.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A compound according to Formula (V): 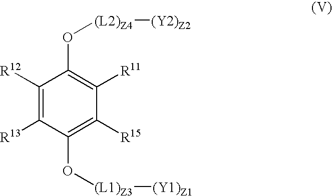
wherein
R¹¹, R¹² and R¹³ are members independently selected from H, halogen, CN, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R¹² and R¹³, together with the carbon atoms to which they are attached, are optionally joined to form a 5- to 7-membered ring;
Z1, Z2, Z3 and Z4 are members independently selected from 0 and 1;
with the proviso that
(i) at least one member selected from Z1 and Z2 is 1;
(ii) when Z4 and Z2 are both 0, (L2)_{Z}4—Y2)_{Z}2 is a member selected from H, a negative charge and a salt counterion; and
(iii) when Z3 and Z1 are both 0, (L1)_{Z}3-(Y1)_{Z}1 is a member selected from H, a negative charge and a salt counterion.
L1 and L2 are independently selected cleavable linker moieties;
Y1 and Y2 are members independently selected from H, a labeling moiety, a targeting moiety, and a drug moiety, with the proviso that at least one of Y1 and Y2 is a drug moiety;
R¹⁵ is a member selected from branched, unsaturated alkyl, —C(O)H, and —CH₂Z^{a} wherein
Z^{a} is a member selected from R¹⁷, OR¹⁷, SR¹⁷, NR¹⁷R¹⁸ and a leaving group
wherein
R¹⁷ and R¹⁸ are members independently selected from substituted or unsubstiluted alkyl, substituted or unsubstituted heteroalkyl, substituted or unsubstituted aryl, substituted or unsubstituted heteroaryl, substituted or unsubstituted cycloalkyl and substituted or unsubstiluted heterocycloalkyl.

2. The compound according to claim 1, wherein said labeling moiety is a member selected from a photoaffinity label, an MRI agent, a fluorescent label, a chemoluminescent label, a histochemical staining reagent, a dye, a moiety for colorimetric detection, and a radioactive label.

3. The compound according to claim 1, wherein the targeting moiety is a member selected from a carbohydrate, a lipid, a peptide, a protein, an antibody, and a small-molecule ligand.

4. The compound according to claim 1, comprising a moiety, which is a member selected from: 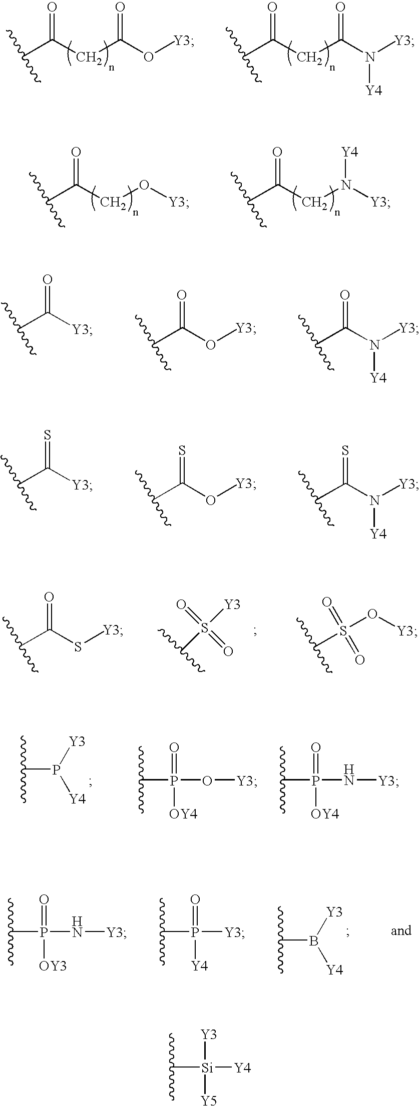
wherein
Y3 is a member selected from Y1 and Y2;
Y4 and Y5 are members independently selected from H, substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl.

5. The compound according to claim 1, wherein R¹² and R¹³ are members independently selected from substituted or unsubstituted C₁-C₆ alkyl and substituted or unsubstituted C₁-C₆ alkoxy.

6. The compound according to claim 5, wherein R¹¹ is a member selected from H and methyl.

7. The compound according to claim 1, wherein R¹⁵ comprises a structure according to Formula (VI): 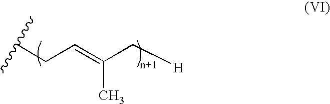
wherein
n is an integer selected from 0 to 13.

8. The compound according to claim 7, wherein n is an integer selected from 3 to 9.

9. The compound according to claim 1, wherein the drug moiety is a member selected from a CETP inhibitor, a statin, a bile acid resin, a fibrate, nicotinic acid, dopamine, retinoic acid, azeleic acid, adapalene, salicylic acid, glycolic acid, benzoyl peroxide, erythromycin, neosporin, polysporin, benzalkonium chloride, chlorhexidine, hexachlorophine, fusidic acid, neomycin, oxytetracyclin, mupirocin, flucloxacillin, doxorubicin, acyclovir, tazarotene, olanzapine, amlopdipine, entacapone, tolcapone, carbidopa, levodopa, benztropine, bromocriptine, donepezil, tacrine, rivastigmine, galantamine, memantine, zolpidem, lorazepam, flurazepam, zaleplon, paroxetine, venlafaxine, fluoxetine, buproprion, sertraline, alprazolam, citalopram, methylphenidate, imipramine, amitryptiline, amoxapine, taxol and taxotere.

10. The compound according to claim 9, wherein the bile acid resin is a member selected from cholestyramine, colestipol and colesevelam.

11. The compound according to claim 9, wherein the fibrate is a member selected from gemfibrozil and fenofibrate.

12. The compound according to claim 9, wherein the statin is a member selected from atorvastatin, fluvastatin, lovastatin, pravastatin, rosuvastatin and simvastatin.

13. The compound according to claim 9, wherein the CETP inhibitor is a member selected from torcetrapib and ezetimibe.

14. The compound according to claim 1, having a structure which is a member selected from: 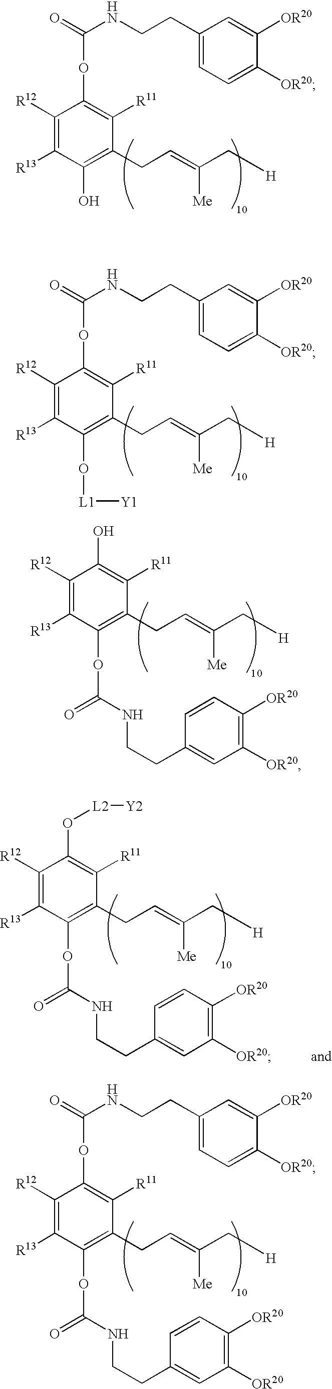
wherein
R²⁰ is a member selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

15. A pharmaceutical composition comprising a compound of claim 1, or a pharmaceutically acceptable salt thereof, and a pharmaceutically acceptable carrier.

16. A method for treating a neurological disorder, said method comprising administering to a subject in need of such treatment, a therapeutically effective amount of a compound according to claim 1 or a pharmaceutically acceptable salt or solvate thereof.

17. The method of claim 16, wherein said neurological disorder is a member selected from central nervous system ischemia, central nervous system trauma, epilepsy, seizures, neurodegenerative diseases, vascular dementia, manic-depressive psychosis, depression, schizophrenia, chronic pain, trigeminal neuralgia, migraine, ataxia, bipolar disorder, spasticity, mood disorders, psychotic disorders, hearing and vision loss, age-related memory loss, learning deficiencies, anxiety and cerebral edema.

18. The method of claim 17, wherein said central nervous system trauma is a member selected from brain trauma, spinal cord injury and whiplash injury.

19. The method of claim 17, wherein said neurodegenerative disease is a member selected from amyotrophic lateral sclerosis (ALS), Alzheimer's disease, Huntington's disease, Parkinson's disease and diabetic neuropathy.

20. The method of claim 17, wherein said vascular dementia is a member selected from multi-infarct dementia and Binswanger's disease.

21. A method of increasing the concentration of a drug molecule in the tissue of the brain, said method comprising administering to a subject in need of such treatment, a therapeutically effective amount of a compound according to claim 1 or a pharmaceutically acceptable salt or solvate thereof, wherein said compound comprises said drug molecule.

22. A method of enhancing the therapeutic activity of a drug molecule, said method comprising:
(a) providing a compound according to claim 1, wherein said compound comprises said drug molecule;
(b) administering to a subject in need of such treatment, a therapeutically effective amount of said compound or a pharmaceutically acceptable salt or solvate thereof.

23. A method of lowering cholesterol concentration in the bloodstream of a patient, comprising administering to a subject in need of such treatment, a therapeutically effective amount of a compound according to claim 1 or a pharmaceutically acceptable salt or solvate thereof, wherein at least one of said drug moieties is a member selected from a CETP inhibitor and a statin.

24. A method of lowering low-density lipoprotein (LDL) concentration in the bloodstream of a patient, comprising administering to a subject in need of such treatment, a therapeutically effective amount of a compound according to claim 1 or a pharmaceutically acceptable salt or solvate thereof, wherein at least one of said drug moieties is a statin.
